(19) United States
(12) Patent Application Publication
(10) Pub. No.: US 2024/0199586 A1
(43) Pub. Date: Jun. 20, 2024

(54) COMPOUND OR TAUTOMER THEREOF, COMPOSITION, LAMINATE, OPTICAL FILM, IMAGE FORMING MATERIAL, AND METHOD FOR PRODUCING COMPOUND OR TAUTOMER THEREOF

(71) Applicant: FUJIFILM Corporation, Tokyo (JP)

(72) Inventor: Yoshihiro JIMBO, Shizuoka (JP)

(73) Assignee: FUJIFILM Corporation, Tokyo (JP)

(21) Appl. No.: 18/430,679

(22) Filed: Feb. 2, 2024

Related U.S. Application Data
(63) Continuation of application No. PCT/JP2022/020721, filed on May 18, 2022.

(30) Foreign Application Priority Data
Aug. 17, 2021 (JP) 2021-133007

(51) Int. Cl. C07D 403/14; C03C 17/32; C07D 401/14; C07D 417/14

(52) U.S. Cl. CPC C07D 403/14; C03C 17/32; C07D 401/14; C07D 417/14

(57) ABSTRACT

A compound represented by Formula (1) or a tautomer thereof; a composition; a laminate; an optical film; an image forming material; and a method for producing a compound or tautomer thereof [R¹ to R⁴: a hydrogen atom or the like, R²⁰: a group represented by Formula (2) or the like, A: an aromatic hydrocarbon group or the like, B: a cationic aromatic hydrocarbon group or the like, *: a bonding position, with the proviso that a compound corresponding to [a], a compound corresponding to [b], and tautomers thereof are excluded. [a] A: a group represented by Formula (P), R²⁰: a hydrogen atom, *: a bonding position, R¹ and R³ are the same, and R² and R⁴ are the same. [b] A: a group represented by Formula (P), R²⁰: a group represented by Formula (2), *: a bonding position, B: a group represented by Formula (Q), R¹ and R³ are the same, and R² and R⁴ are the same.].

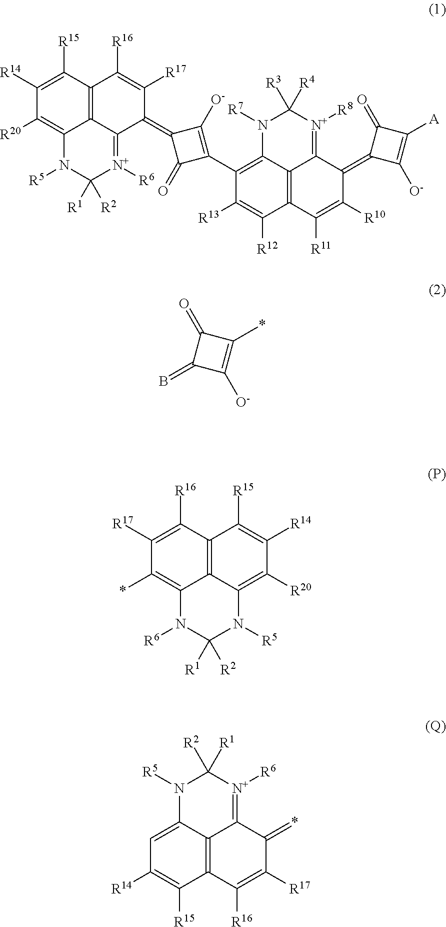

### CROSS-REFERENCE TO RELATED APPLICATIONS

This application is a continuation application of International Application No. PCT/JP2022/020721, filed May 18, 2022, the disclosure of which is incorporated herein by reference in its entirety. Further, this application claims priority from Japanese Patent Application No. 2021-133007, filed Aug. 17, 2021, the disclosure of which is incorporated herein by reference in its entirety.

### BACKGROUND OF THE INVENTION

### 1. Field of the Invention

The present disclosure relates to a compound or a tautomer thereof, a composition, a laminate, an optical film, an image forming material, and a method for producing a compound or a tautomer thereof.

### 2. Description of the Related Art

In the related art, a dihydroperimidine squarylium compound having a squarylium skeleton is known (for example, see U.S. Pat. No. 5,380,635A). The dihydroperimidine squarylium compound is a compound having maximum absorption in a wavelength range of 780 nm to 830 nm and is useful as a near-infrared absorbing material. In addition, a squarylium compound having two or more squarylium skeletons and having maximum absorption in a longer wavelength range has also been reported (for example, see JP1998-204310A (JP-H10-204310A) and WO2007/091683A).

### SUMMARY OF THE INVENTION

In recent years, there has been a demand for the near-infrared absorbing material to be able to sufficiently absorb light having a longer wavelength, and for example, there has been a desire to develop a compound having maximum absorption in a near-infrared range on a longer wavelength side than 850 nm.

In this regard, the squarylium compound described in U.S. Pat. No. 5,380,635A has maximum absorption in a wavelength range of 780 nm to 830 nm, so it cannot be said that the absorption of light in a wavelength range longer than 850 nm is sufficient. On the other hand, the squarylium compound described in JP1998-204310A (JP-H10-204310A) and WO2007/091683A has maximum absorption in a near-infrared range on a longer wavelength side than 850 nm, and is therefore presumed to be able to sufficiently absorb light in a wavelength range longer than 850 nm. However, according to the research conducted by the present inventors, it has been found that the squarylium compounds described in JP1998-204310A (JP-H10-204310A) and WO2007/091683A are colored in a visible range, and it has been found that the squarylium compound described in JP1998-204310A (JP-H10-204310A) tends to have poor light resistance, and in a case where the squarylium compound is exposed to light, the absorption ability thereof for light having a wavelength in a near-infrared range is significantly reduced.

An object to be achieved by an embodiment of the present disclosure is to provide a novel compound or a tautomer thereof, which is suppressed from coloring in a visible range, has maximum absorption in a near-infrared range on a longer wavelength side than 850 nm, and has excellent light resistance.

An object to be achieved by another embodiment of the present disclosure is to provide a composition containing the above-described compound or the tautomer thereof, a laminate, an optical filter, and an image forming material.

An object to be achieved by still another embodiment of the present disclosure is to provide a method for producing the above-described compound or the tautomer thereof.

Specific means for achieving the above-described objects include the following embodiments.
<1> A compound represented by Formula (1) or a tautomer thereof.

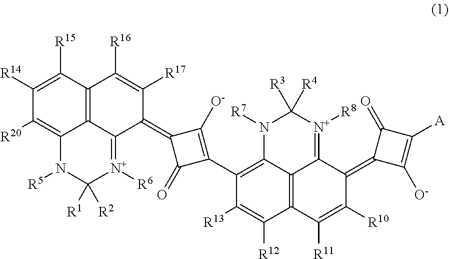

In Formula (1), R¹, R², R³, and R⁴ each independently represent a hydrogen atom, an alkyl group, or an aryl group, R⁵, R⁶, R⁷, and R⁸ each independently represent a hydrogen atom, an alkyl group, or an aryl group, R¹⁰, R¹¹, R¹², R¹³, R¹⁴, R¹⁵, R¹⁶, and R¹⁷ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, R²⁰ represents a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, an arylthio group, or a group represented by Formula (2), and A represents an aromatic hydrocarbon group, a heterocyclic group, or a heterocyclic methine group.

In Formula (2), B represents a cationic aromatic hydrocarbon group, a cationic heterocyclic group, or a cationic heterocyclic methine group, and * represents a bonding position.

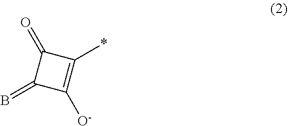

In Formula (1), R¹ and R², and R³ and R⁴ each may be bonded to each other to form a ring, R¹ and R⁵, R² and R⁶, R³ and R⁷, R⁴ and R⁸ each may be bonded to each other to form a ring, and R¹⁰, R¹¹, R¹², R¹³, R¹⁴, R¹⁵, R¹⁶, R¹⁷, and R²⁰ each may be bonded to an adjacent group to form a ring.

In this regard, a compound corresponding to the following [a], a compound corresponding to the following [b], and tautomers thereof are excluded.
(a) In Formula (1), A is a group represented by Formula (P), R²⁰ is a hydrogen atom, R¹ and R³ are the same, and R² and R⁴ are the same.
(b) In Formula (1), A is a group represented by Formula (P), R²⁰ is a group represented by Formula (2), B is a group represented by Formula (Q), R¹ and R³ are the same, and R² and R⁴ are the same.

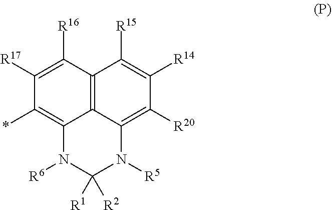

In Formula (P), R¹ and R² each independently represent a hydrogen atom, an alkyl group, or an aryl group, R⁵ and R⁶ each independently represent a hydrogen atom, an alkyl group, or an aryl group, R¹⁴, R¹⁵, R¹⁶, and R¹⁷ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, R²⁰ represents a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, an arylthio group, or a group represented by Formula (2), and * represents a bonding position.

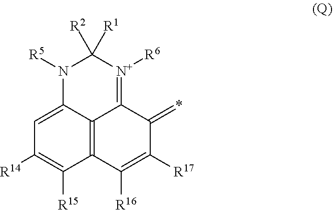

In Formula (Q), R¹ and R² each independently represent a hydrogen atom, an alkyl group, or an aryl group, R⁵ and R⁶ each independently represent a hydrogen atom, an alkyl group, or an aryl group, R¹⁴, R¹⁵, R¹⁶, and R¹⁷ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, and * represents a bonding position.
<2> The compound or tautomer thereof according to <1>, in which A in Formula (1) is a group represented by Formula (A-1), a group represented by Formula (A-2), a group represented by Formula (A-3), a group represented by Formula (A-4), or a group represented by Formula (A-5).

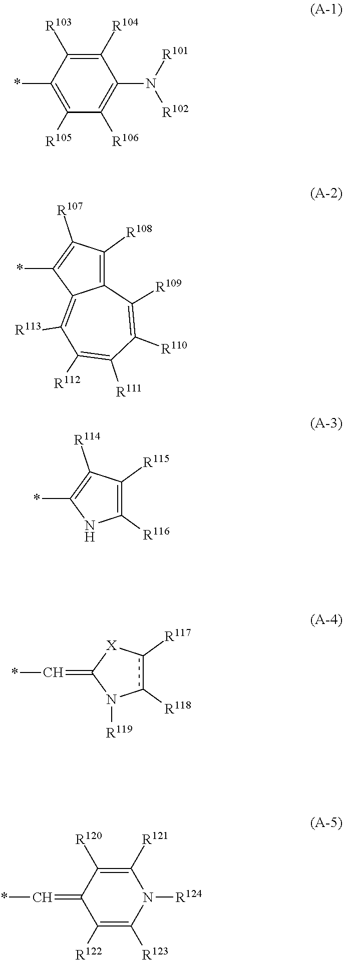

In Formula (A-1), Formula (A-2), Formula (A-3), Formula (A-4), and Formula (A-5), R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹, R¹¹², R¹¹³, R¹¹⁴, R¹¹⁵, R¹¹⁶, R¹¹⁷, R¹¹⁸, R¹¹⁹, R¹²⁰, R¹²¹, R¹²², R¹²³, and R¹²⁴ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, X represents —O—, —S—, —CH═CH—, or —CR¹²⁵R¹²⁶— where R¹²⁵ and R¹²⁶ each independently represent an alkyl group, a bond between the carbon atom to which R¹¹⁷ is bonded and the carbon atom to which R¹¹⁸ is bonded represents a single bond or a double bond, * represents a bonding position, and R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹, R¹¹², R¹¹³, R¹¹⁴, R¹¹⁵, R¹¹⁶, R¹¹⁷, R¹¹⁸, R¹¹⁹, R¹²⁰, R¹²¹, R¹²², R¹²³, R¹²⁴, R¹²⁵, and R¹²⁶ each may be bonded to an adjacent group to form a ring.
<3> The compound or tautomer thereof according to <1> or <2>, in which B in Formula (2) is a group represented by Formula (B-1), a group represented by Formula (B-2), a group represented by Formula (B-3), a group represented by Formula (B-4), or a group represented by Formula (B-5).

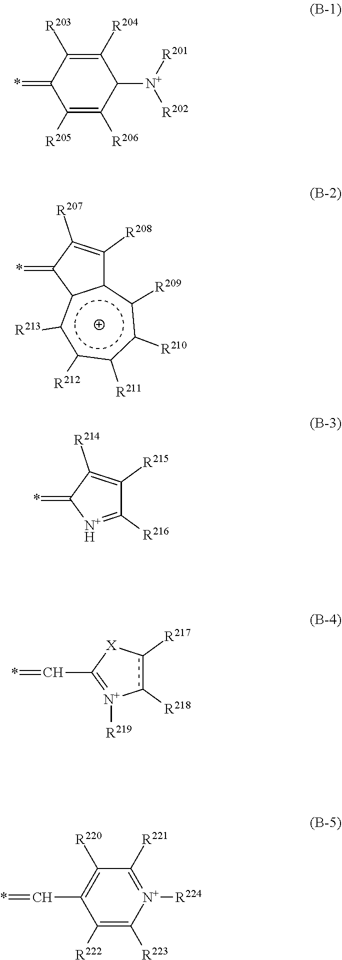

In Formula (B-1), Formula (B-2), Formula (B-3), Formula (B-4), and Formula (B-5), R²⁰¹, R²⁰², R²⁰³, R²⁰⁴, R²⁰⁵, R²⁰⁶, R²⁰⁷, R²⁰⁸, R²⁰⁹, R²¹⁰, R²¹¹, R²¹², R²¹³, R²¹⁴, R²¹⁵, R²¹⁶, R²¹⁷, R²¹⁸, R²¹⁹, R²²⁰, R²²¹, R²²², R²²³, and R²²⁴ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, X represents —O—, —S—, —CH═CH—, or —CR²²⁵R²²⁶— where R²²⁵ and R²²⁶ each independently represent an alkyl group, a bond between the carbon atom to which R²¹⁷ is bonded and the carbon atom to which R²¹⁸ is bonded represents a single bond or a double bond, * represents a bonding position, and R²⁰¹, R²⁰², R²⁰³, R²⁰⁴, R²⁰⁵, R²⁰⁶, R²⁰⁷, R²⁰⁸, R²⁰⁹, R²¹⁰, R²¹¹, R²¹², R²¹³, R²¹⁴, R²¹⁵, R²¹⁶, R²¹⁷, R²¹⁸, R²¹⁹, R²²⁰, R²²¹, R²²², R²²³, R²²⁴, R²²⁵, and R²²⁶ each may be bonded to an adjacent group to form a ring.
<4> The compound or tautomer thereof according to any one of <1> to <3>, in which R⁵, R⁶, R⁷, and R⁸ in Formula (1) each represent a hydrogen atom.
<5> The compound or tautomer thereof according to any one of <1> to <4>, in which R¹⁰, R¹¹, R¹², R¹³, R¹⁴, R¹⁵, R¹⁶, and R¹⁷ in Formula (1) each represent a hydrogen atom.
<6> The compound or tautomer thereof according to any one of <1> to <5>, in which R²⁰ in Formula (1) represents a hydrogen atom or a group represented by Formula (2).
<7> A composition including the compound or tautomer thereof according to any one of <1> to <6>.
<8> The composition according to <7>, further including a resin.
<9> The composition according to <7> or <8>, in which the composition is a curable composition.
<10> The composition according to any one of <7> to <9>, in which the composition is a near-infrared absorbing material.
<11> A laminate including a support and a film that is provided on the support and is formed of the composition according to any one of <7> to <10>.
<12> An optical filter including the compound or tautomer thereof according to any one of <1> to <6>.
<13> An image forming material including the compound or tautomer thereof according to any one of <1> to <6>.
<14> A method for producing the compound or tautomer thereof according to any one of <1> to <6>, the method including:
   reacting a compound represented by Formula (3) with a compound represented by Formula (4).

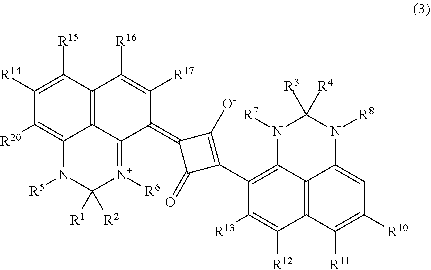

In Formula (3), R¹, R², R³, and R⁴ each independently represent a hydrogen atom, an alkyl group, or an aryl group, R⁵, R⁶, R⁷, and R⁸ each independently represent a hydrogen atom, an alkyl group, or an aryl group, R¹⁰, R¹¹, R¹², R¹³, R¹⁴, R¹⁵, R¹⁶, and R¹⁷ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, and R²⁰ represents a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, an arylthio group, or a group represented by Formula (2).

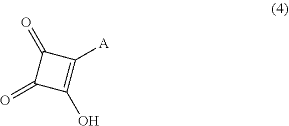

In Formula (4), A represents an aromatic hydrocarbon group, a heterocyclic group, or a heterocyclic methine group.
<15> The method for producing the compound or tautomer thereof according to <14>, in which A in Formula (4) is a group represented by Formula (A-1), R²⁰ in Formula (3) is a group represented by Formula (2), and B in Formula (2) is a group represented by Formula (B-1), or
   A in Formula (4) is a group represented by Formula (A-2), R²⁰ in Formula (3) is a group represented by Formula (2), and B in Formula (2) is a group represented by Formula (B-2), or
   A in Formula (4) is a group represented by Formula (A-3), R²⁰ in Formula (3) is a group represented by Formula (2), and B in Formula (2) is a group represented by Formula (B-3), or
   A in Formula (4) is a group represented by Formula (A-4), R²⁰ in Formula (3) is a group represented by Formula (2), and B in Formula (2) is a group represented by Formula (B-4), or
   A in Formula (4) is a group represented by Formula (A-5), R²⁰ in Formula (3) is a group represented by Formula (2), and B in Formula (2) is a group represented by Formula (B-5).

According to an embodiment of the present disclosure, there is provided a novel compound or a tautomer thereof, which is suppressed from coloring in a visible range, has maximum absorption in a near-infrared range on a longer wavelength side than 850 nm, and has excellent light resistance.

According to another embodiment of the present disclosure, there are provided a composition containing the above-described compound or the tautomer thereof, a laminate, an optical filter, and an image forming material.

According to still another embodiment of the present disclosure, there is provided a method for producing the above-described compound or the tautomer thereof.

### DESCRIPTION OF THE PREFERRED EMBODIMENTS

Hereinafter, the present disclosure will be described in detail. The description of the requirements set forth below may be based on representative embodiments of the present disclosure, but the present disclosure is not limited to such embodiments. Within the scope of the purpose of the present disclosure, modifications can be made as appropriate.

In the present disclosure, any numerical range indicated using “to” means a range including numerical values described before and after “to” as a lower limit value and an upper limit value, respectively.

In any numerical range described in a stepwise manner in the present disclosure, an upper limit value or a lower limit value described in a certain numerical range may be replaced with an upper limit value or a lower limit value of another numerical range described in a stepwise manner. In addition, in any numerical range described in the present disclosure, an upper limit value or a lower limit value described in a certain numerical range may be replaced with values shown in Examples.

In the present disclosure, upon referring to an amount of each component in a composition, the amount means a total amount of a plurality of components present in the composition unless otherwise specified, in a case where a plurality of substances corresponding to individual components are present in the composition.

In the present disclosure, a combination of two or more preferred aspects is a more preferred aspect.

In the present disclosure, the term “solid content” refers to components excluding a solvent, and the term “solvent” refers to water and an organic solvent.

In the present disclosure, the term “step” includes not only an independent step, but also a step that may not be clearly distinguished from other steps but still achieves a desired effect of the step.

In the present disclosure, “(meth)acrylic” is a term including both “acrylic” and “methacrylic”, “(meth)acrylate” is a term including both “acrylate” and “methacrylate”, and “(meth)acryloyl” is a term including both “acryloyl” and “methacryloyl”.

In the present disclosure, “monomeric substance” and “monomer” have the same definition.

In the present disclosure, “n-” refers to “normal”.

In the present disclosure, the molecular weight in a case where there is a molecular weight distribution represents a weight-average molecular weight (Mw; the same applies hereinafter) unless otherwise specified.

The weight-average molecular weight (Mw) in the present disclosure is a value measured by gel permeation chromatography (GPC).

The measurement by GPC is carried out using HLC (registered trademark)-8220GPC [manufactured by Tosoh Corporation] as a measuring device, three columns of TSKgel (registered trademark) Super HZ2000 [4.6 mm ID×15 cm, manufactured by Tosoh Corporation], TSKgel (registered trademark) Super HZ4000 [4.6 mm ID×15 cm, manufactured by Tosoh Corporation] and TSKgel (registered trademark) Super HZ-H [4.6 mm ID×15 cm, manufactured by Tosoh Corporation] which are connected in series, and N-methylpyrrolidone (NMP) as an eluent. In addition, a differential refractive index (RI) detector as a detector is used to carry out GPC under the measurement conditions: a sample concentration of 0.3% by mass, a flow rate of 0.35 ml/min, a sample injection volume of 10 μL, and a measurement temperature of 40° C. The calibration curve is prepared from 6 samples of “F-80”, “F-20”, “F-4”, “F-2”, “A-5000”, and “A-1000”, which are “Standard Sample TSK standard, polystyrene” manufactured by Tosoh Corporation.

The “substituent” in the present disclosure is not particularly limited and can be optionally selected from the following substituent group.

Substituent group: a halogen atom, an alkyl group, an alkenyl group, an alkynyl group, an aryl group, a heterocyclic group, a cyano group, a hydroxy group, a nitro group, a carboxy group, an alkoxy group, an aryloxy group, a heterocyclic oxy group, an acyloxy group, a carbamoyloxy group, an alkoxycarbonyloxy group, an aryloxycarbonyloxy group, an amino group, an acylamino group, an aminocarbonylamino group, an alkoxycarbonylamino group, an aryloxycarbonylamino group, a sulfamoylamino group, an alkylsulfonylamino group, an arylsulfonylamino group, a mercapto group, an alkylthio group, an arylthio group, a heterocyclic thio group, a sulfamoyl group, a sulfo group, an alkylsulfinyl group, an arylsulfinyl group, an alkylsulfonyl group, an arylsulfonyl group, an acyl group, an aryloxycarbonyl group, an alkoxycarbonyl group, a carbamoyl group, an arylazo group, a heterocyclic azo group, an imide group, a phosphino group, a phosphinyl group, a phosphinyloxy group, a phosphinylamino group, and a silyl group.

### [Compound Represented by Formula (1) or Tautomer Thereof]

The compound according to the embodiment of the present disclosure is a compound represented by Formula (1) or a tautomer thereof.

The compound according to the embodiment of the present disclosure is a novel compound which is suppressed from coloring in a visible range, has maximum absorption in a near-infrared range on a longer wavelength side than 850 nm, and has excellent light resistance.

The “tautomer” refers to, for example, a compound that exists as two or more isomers which can be readily interconverted from one to the other. Examples of the tautomer include an isomer that results from the transfer of a proton bonded to one atom in a molecule to another atom, and an isomer that results from the transfer of a localized electric charge on a specific atom in a molecule to another atom.

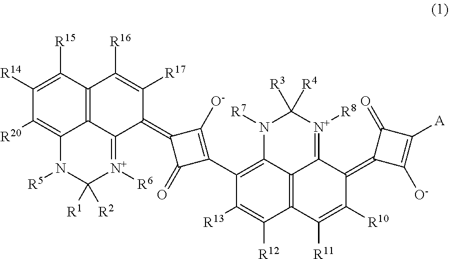

In Formula (1), R¹, R², R³, and R⁴ each independently represent a hydrogen atom, an alkyl group, or an aryl group, R⁵, R⁶, R⁷, and R⁸ each independently represent a hydrogen atom, an alkyl group, or an aryl group, R¹⁰, R¹¹, R¹², R¹³, R¹⁴, R¹⁵, R¹⁶, and R¹⁷ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, R²⁰ represents a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, an arylthio group, or a group represented by Formula (2), and A represents an aromatic hydrocarbon group, a heterocyclic group, or a heterocyclic methine group.

In Formula (2), B represents a cationic aromatic hydrocarbon group, a cationic heterocyclic group, or a cationic heterocyclic methine group, and * represents a bonding position.

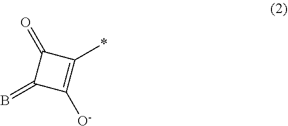

In Formula (1), R¹ and R², and R³ and R⁴ each may be bonded to each other to form a ring, R¹ and R⁵, R² and R⁶, R³ and R⁷, R⁴ and R⁸ each may be bonded to each other to form a ring, and R¹⁰, R¹¹, R¹², R¹³, R¹⁴, R¹⁵, R¹⁶, R¹⁷, and R²⁰ each may be bonded to an adjacent group to form a ring.

In this regard, a compound corresponding to the following [a], a compound corresponding to the following [b], and tautomers thereof are excluded.
[a] In Formula (1), A is a group represented by Formula (P), R²⁰ is a hydrogen atom, R¹ and R³ are the same, and R² and R⁴ are the same.
[b] In Formula (1), A is a group represented by Formula (P), R²⁰ is a group represented by Formula (2), B is a group represented by Formula (Q), R¹ and R³ are the same, and R² and R⁴ are the same.

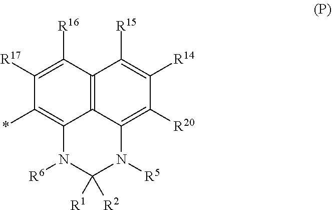

In Formula (P), R¹ and R² each independently represent a hydrogen atom, an alkyl group, or an aryl group, R⁵ and R⁶ each independently represent a hydrogen atom, an alkyl group, or an aryl group, R¹⁴, R¹⁵, R¹⁶, and R¹⁷ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, R²⁰ represents a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, an arylthio group, or a group represented by Formula (2), and * represents a bonding position.

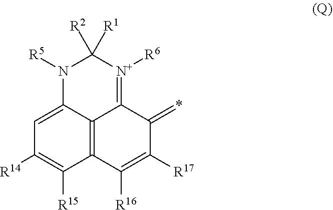

In Formula (Q), R¹ and R² each independently represent a hydrogen atom, an alkyl group, or an aryl group, R⁵ and R⁶ each independently represent a hydrogen atom, an alkyl group, or an aryl group, R¹⁴, R¹⁵, R¹⁶, and R¹⁷ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, and * represents a bonding position.

Hereinafter, the details of the compound represented by Formula (1) or the tautomer thereof will be described.

In Formula (1), R¹, R², R³, and R⁴ each independently represent a hydrogen atom, an alkyl group, or an aryl group.

The alkyl group represented by each of R¹, R², R³, and R⁴ may be a linear alkyl group, an alkyl group having a branch, or an alkyl group having a cyclic structure.

The alkyl group represented by each of R¹, R², R³, and R⁴ is preferably an alkyl group having 1 to 30 carbon atoms and more preferably an alkyl group having 1 to 12 carbon atoms.

The alkyl group represented by each of R¹, R², R³, and R⁴ is, for example, preferably a methyl group, an ethyl group, an n-propyl group, an isopropyl group, a cyclopropyl group, a 2-pentyl group, an n-butyl group, an iso-butyl group, a sec-butyl group, a tert-butyl group, an n-amyl group, an iso-amyl group, an n-hexyl group, an n-octyl group, a 2-ethylhexyl group, an n-nonyl group, an n-decyl group, an undecyl group, a dodecyl group, a hydroxymethyl group, a 2-hydroxyethyl group, a 2-acetyloxyethyl group, a benzyl group, a 4-methylbenzyl group, a cyclohexyl group, or a cyclopentyl group.

The aryl group represented by each of R¹, R², R³, and R⁴ may have a substituent or may not have a substituent.

In a case where the aryl group represented by each of R¹, R², R³, and R⁴ has a substituent, the substituent is, for example, preferably a halogen atom or an alkyl group.

The aryl group represented by each of R¹, R², R³, and R⁴ is preferably an aryl group having 6 to 30 carbon atoms and more preferably an aryl group having 6 to 10 carbon atoms.

Examples of the aryl group represented by each of R¹, R², R³, and R⁴ include a phenyl group, a p-tolyl group, a naphthyl group, an m-chlorophenyl group, and an o-hexadecanoylaminophenyl group. Above all, the aryl group represented by each of R¹, R², R³, and R⁴ is preferably a phenyl group which may have a substituent.

In Formula (1), R¹ and R², and R³ and R⁴ each may be bonded to each other to form a ring.

The ring formed by R¹ and R², and R³ and R⁴ each being bonded to each other is, for example, preferably a saturated or unsaturated carbocyclic ring and more preferably a 5- to 12-membered ring.

Specifically, the ring formed by R¹ and R², and R³ and R⁴ each being bonded to each other is preferably cyclobutane, cyclopentane, cyclohexane, cycloheptane, cyclooctane, cyclododecane, or adamantane, and these rings may have a substituent. The substituent is, for example, preferably a halogen atom or an alkyl group.

Specific examples of a case where the ring formed by R¹ and R², and R³ and R⁴ each being bonded to each other has a substituent include 4-methylcyclohexane, 3-methylcyclohexane, 2-methylcyclohexane, 3,5-dimethylcyclohexane, cyclohexan-4-one, cyclohexan-3-one, indane, and fluorene.

In Formula (1), R⁵, R⁶, R⁷, and R⁸ each independently represent a hydrogen atom, an alkyl group, or an aryl group.

The alkyl group represented by each of R⁵, R⁶, R⁷, and R⁸ may have a substituent or may not have a substituent.

The alkyl group represented by each of R⁵, R⁶, R⁷, and R⁸ may be a linear alkyl group, an alkyl group having a branch, or an alkyl group having a cyclic structure.

The alkyl group represented by each of R⁵, R⁶, R⁷, and R⁸ is preferably an alkyl group having 1 to 30 carbon atoms and more preferably an alkyl group having 1 to 15 carbon atoms.

The alkyl group represented by each of R⁵, R⁶, R⁷, and R⁸ is, for example, preferably a methyl group, an ethyl group, an n-propyl group, an isopropyl group, a tert-butyl group, an n-octyl group, an eicosyl group, a 2-chloroethyl group, a 2-cyanoethyl group, a benzyl group, a 2-ethylhexyl group, a vinyl group, an allyl group, a prenyl group, a geranyl group, an oleyl group, a propargyl group, a cyclohexyl group, a cyclopentyl group, a 2-hydroxyethyl group, a 2-hydroxypropyl group, a 7-trimethoxysilyl-4-oxa-2-hydroxyheptyl group, a 7-triethoxysilyl-4-oxa-2-hydroxyheptyl group, a 2-(3-trimethoxysilylaminocarbonyloxy)ethyl group, a 2-(3-triethoxysilylaminocarbonyloxy)ethyl group, or a 3-trimethoxysilylpropyl group, and more preferably a methyl group, an ethyl group, a 2-ethylhexyl group, a 2-hydroxyethyl group, a 7-trimethoxysilyl-4-oxa-2-hydroxyheptyl group, a 7-triethoxysilyl-4-oxa-2-hydroxyheptyl group, a 2-(3-trimethoxysilylaminocarbonyloxy)ethyl group, or a 2-(3-triethoxysilylaminocarbonyloxy)ethyl group.

The aryl group represented by each of R⁵, R⁶, R⁷, and R⁸ may have a substituent or may not have a substituent.

The substituent that the aryl group represented by each of R⁵, R⁶, R⁷, and R⁸ may have is, for example, preferably a halogen atom or an alkyl group.

The aryl group represented by each of R⁵, R⁶, R⁷, and R⁸ is preferably an aryl group having 6 to 30 carbon atoms and more preferably an aryl group having 6 to 10 carbon atoms.

Examples of the aryl group represented by each of R⁵, R⁶, R⁷, and R⁸ include a phenyl group, a p-tolyl group, a naphthyl group, an m-chlorophenyl group, and an o-hexadecanoylaminophenyl group. Above all, the aryl group represented by each of R⁵, R⁶, R⁷, and R⁸ is preferably a phenyl group which may have a substituent.

In Formula (1), R¹ and R⁵, R² and R⁶, R³ and R⁷, and R⁴ and R⁸ each may be bonded to each other to form a ring.

The ring formed by R¹ and R⁵, R² and R⁶, R³ and R⁷, and R⁴ and R⁸ each being bonded to each other is, for example, preferably a nitrogen-containing 5-membered ring or a nitrogen-containing 6-membered ring.

Specifically, the ring formed by R¹ and R⁵, R² and R⁶, R³ and R⁷, and R⁴ and R⁸ each being bonded to each other is, for example, preferably pyrrolidine or piperidine.

In Formula (1), R⁵, R⁶, R⁷, and R⁸ are each particularly preferably a hydrogen atom, for example, from the viewpoint of light resistance.

In Formula (1), R¹⁰, R¹¹, R¹², R¹³, R¹⁴, R¹⁵, R¹⁶, and R¹⁷ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group.

The halogen atom represented by each of R¹⁰, R¹¹, R¹², R¹³, R¹⁴, R¹⁵, R¹⁶, and R¹⁷ is, for example, preferably a fluorine atom, a chlorine atom, a bromine atom, or an iodine atom, and more preferably a fluorine atom or a chlorine atom.

The alkyl group represented by each of R¹⁰, R¹¹, R¹², R¹³, R¹⁴, R¹⁵, R¹⁶, and R¹⁷ may have a substituent or may not have a substituent.

The alkyl group represented by each of R¹⁰, R¹¹, R¹², R¹³, R¹⁴, R¹⁵, R¹⁶, and R¹⁷ may be a linear alkyl group, an alkyl group having a branch, or an alkyl group having a cyclic structure.

The alkyl group represented by each of R¹⁰, R¹¹, R¹², R¹³, R¹⁴, R¹⁵, R¹⁶, and R¹⁷ is preferably an alkyl group having 1 to 30 carbon atoms and more preferably an alkyl group having 1 to 15 carbon atoms.

The alkyl group represented by each of R¹⁰, R¹¹, R¹², R¹³, R¹⁴, R¹⁵, R¹⁶, and R¹⁷ is, for example, preferably a methyl group, an ethyl group, an n-propyl group, an isopropyl group, a tert-butyl group, an n-octyl group, an eicosyl group, a benzyl group, a 2-ethylhexyl group, an allyl group, a prenyl group, a geranyl group, an oleyl group, a propargyl group, a cyclohexyl group, a cyclopentyl group, a 2-hydroxyethyl group, a 7-trimethoxysilyl-4-oxa-2-hydroxyheptyl group, a 7-triethoxysilyl-4-oxa-2-hydroxyheptyl group, a 2-(3-trimethoxysilylaminocarbonyloxy)ethyl group, a 2-(3-triethoxysilylaminocarbonyloxy)ethyl group, or a 3-trimethoxysilylpropyl group, and more preferably a methyl group, an ethyl group, a 2-ethylhexyl group, a 2-hydroxyethyl group, a 7-trimethoxysilyl-4-oxa-2-hydroxyheptyl group, a 7-triethoxysilyl-4-oxa-2-hydroxyheptyl group, a 2-(3-trimethoxysilylaminocarbonyloxy)ethyl or a group, 2-(3-triethoxysilylaminocarbonyloxy)ethyl group.

The aryl group represented by each of R¹⁰, R¹¹, R¹², R¹³, R¹⁴, R¹⁵, R¹⁶, and R¹⁷ may have a substituent or may not have a substituent.

The substituent that the aryl group represented by each of R¹⁰, R¹¹, R¹², R¹³, R¹⁴, R¹⁵, R¹⁶, and R¹⁷ may have is, for example, preferably a halogen atom, an alkyl group, or an alkoxy group.

The aryl group represented by each of R¹⁰, R¹¹, R¹², R¹³, R¹⁴, R¹⁵, R¹⁶, and R¹⁷ is preferably an aryl group having 6 to 30 carbon atoms and more preferably an aryl group having 6 to 10 carbon atoms.

Specific examples of the aryl group represented by each of R¹⁰, R¹¹, R¹², R¹³, R¹⁴, R¹⁵, R¹⁶, and R¹⁷ include a phenyl group, a p-tolyl group, a naphthyl group, an m-chlorophenyl group, and an o-hexadecanoylaminophenyl group. Above all, the aryl group represented by each of R¹⁰, R¹¹, R¹², R¹³, R¹⁴, R¹⁵, R¹⁶, and R¹⁷ is preferably a phenyl group which may have a substituent.

The alkoxy group represented by each of R¹⁰, R¹¹, R¹², R¹³, R¹⁴, R¹⁵, R¹⁶, and R¹⁷ may be a linear alkoxy group, an alkoxy group having a branch, or an alkoxy group having a cyclic structure.

The alkoxy group represented by each of R¹⁰, R¹¹, R¹², R¹³, R¹⁴, R¹⁵, R¹⁶, and R¹⁷ is preferably an alkoxy group having 1 to 30 carbon atoms.

The alkoxy group represented by each of R¹⁰, R¹¹, R¹², R¹³, R¹⁴, R¹⁵, R¹⁶, and R¹⁷ is, for example, preferably a methoxy group, an ethoxy group, an n-propoxy group, or a tert-butoxy group.

The aryloxy group represented by each of R¹⁰, R¹¹, R¹², R¹³, R¹⁴, R¹⁵, R¹⁶, and R¹⁷ may have a substituent or may not have a substituent.

The substituent that the aryloxy group represented by each of R¹⁰, R¹¹, R¹², R¹³, R¹⁴, R¹⁵, R¹⁶, and R¹⁷ may have is, for example, preferably a halogen atom or an alkyl group.

The aryloxy group represented by each of R¹⁰, R¹¹, R¹², R¹³, R¹⁴, R¹⁵, R¹⁶, and R¹⁷ is preferably an aryloxy group having 6 to 30 carbon atoms.

The aryloxy group represented by each of R¹⁰, R¹¹, R¹², R¹³, R¹⁴, R¹⁵, R¹⁶, and R¹⁷ is, for example, preferably a phenoxy group, a 4-chlorophenoxy group, or a 4-methylphenoxy group.

The acyloxy group represented by each of R¹⁰, R¹¹, R¹², R¹³, R¹⁴, R¹⁵, R¹⁶, and R¹⁷ is preferably an acyloxy group having 2 to 30 carbon atoms.

The acyloxy group represented by each of R¹⁰, R¹¹, R¹², R¹³, R¹⁴, R¹⁵, R¹⁶, and R¹⁷ is, for example, preferably an acetyloxy group, a propionyloxy group, a pivaloyloxy group, a 2-ethylhexanoyloxy group, a benzoyloxy group, or a 4-methoxyoxy group.

The alkoxycarbonyl group represented by each of R¹⁰, R¹¹, R¹², R¹³, R¹⁴, R¹⁵, R¹⁶, and R¹⁷ is, for example, preferably an alkoxycarbonyl group whose alkoxy moiety has 1 to 30 carbon atoms, suitable examples of which include a methoxycarbonyl group, an ethoxycarbonyl group, and a 2-ethoxyethoxycarbonyl group.

The carbamoyl group represented by each of R¹⁰, R¹¹, R¹², R¹³, R¹⁴, R¹⁵, R¹⁶, and R¹⁷ may have a substituent or may not have a substituent.

The substituent that the carbamoyl group represented by each of R¹⁰, R¹¹, R¹², R¹³, R¹⁴, R¹⁵, R¹⁶, and R¹⁷ may have is, for example, preferably an alkyl group.

The carbamoyl group represented by each of R¹⁰, R¹¹, R¹², R¹³, R¹⁴, R¹⁵, R¹⁶, and R¹⁷ is preferably a carbamoyl group having 1 to 30 carbon atoms and more preferably a carbamoyl group having 1 to 15 carbon atoms.

Specific examples of the carbamoyl group represented by each of R¹⁰, R¹¹, R¹², R¹³, R¹⁴, R¹⁵, R¹⁶, and R¹⁷ include an N,N-dimethylcarbamoyl group, an N,N-diethylcarbamoyl group, a morpholinocarbonyl group, an N,N-di-n-octylaminocarbonyl group, an N-n-octylcarbamoyl group, an N-3-trimethoxysilylpropylcarbamoyl group, and an N-3-triethoxysilylpropylcarbamoyl group.

The carbamoyl group represented by each of R¹⁰, R¹¹, R¹², R¹³, R¹⁴, R¹⁵, R¹⁶, and R¹⁷ is, for example, preferably an N,N-dimethylcarbamoyl group, an N,N-diethylcarbamoyl group, an N-3-trimethoxysilylpropylcarbamoyl group, or an N-3-triethoxysilylpropylcarbamoyl group.

The acyl group represented by each of R¹⁰, R¹¹, R¹², R¹³, R¹⁴, R¹⁵, R¹⁶, and R¹⁷ is preferably an acyl group having 2 to 30 carbon atoms and more preferably an acyl group having 2 to 15 carbon atoms.

Specific examples of the acyl group represented by each of R¹⁰, R¹¹, R¹², R¹³, R¹⁴, R¹⁵, R¹⁶, and R¹⁷ include an acetyl group, a pivaloyl group, a 2-ethylhexanoyl group, a stearoyl group, a benzoyl group, and a p-methoxybenzoyl group. Above all, the acyl group represented by each of R¹⁰, R¹¹, R¹², R¹³, R¹⁴, R¹⁵, R¹⁶, and R¹⁷ is, for example, preferably an acetyl group, a pivaloyl group, or a 2-ethylhexanoyl group.

The amino group represented by each of R¹⁰, R¹¹, R¹², R¹³, R¹⁴, R¹⁵, R¹⁶, and R¹⁷ is, for example, preferably an unsubstituted amino group, a primary amino group, or a secondary amino group and more preferably a methylamino group, a dimethylamino group, an ethylamino group, a diethylamino group, a pyrrolidino group, a piperidino group, or a morpholino group.

The amide group represented by each of R¹⁰, R¹¹, R¹², R¹³, R¹⁴, R¹⁵, R¹⁶, and R¹⁷ may have a substituent or may not have a substituent.

The substituent that the amide group represented by each of R¹⁰, R¹¹, R¹², R¹³, R¹⁴, R¹⁵, R¹⁶, and R¹⁷ may have is, for example, preferably an alkyl group.

The amide group represented by each of R¹⁰, R¹¹, R¹², R¹³, R¹⁴, R¹⁵, R¹⁶, and R¹⁷ is, for example, preferably a formamide group, an acetamide group, a propionylamino group, a benzamide group, or a phthalimide group.

The carbamoylamino group represented by each of R¹⁰, R¹¹, R¹², R¹³, R¹⁴, R¹⁵, R¹⁶, and R¹⁷ may have a substituent or may not have a substituent.

The substituent that the carbamoylamino group represented by each of R¹⁰, R¹¹, R¹², R¹³, R¹⁴, R¹⁵, R¹⁶, and R¹⁷ may have is, for example, preferably an alkyl group.

The carbamoylamino group represented by each of R¹⁰, R¹¹, R¹², R¹³, R¹⁴, R¹⁵, R¹⁶, and R¹⁷ is, for example, preferably a dimethylcarbamoylamino group or a diethylcarbamoylamino group.

The alkoxycarbonylamino group represented by each of R¹⁰, R¹¹, R¹², R¹³, R¹⁴, R¹⁵, R¹⁶, and R¹⁷ is, for example, preferably an alkoxycarbonylamino group whose alkoxy moiety has 1 to 30 carbon atoms, suitable examples of which include a methoxycarbonylamino group and an ethoxycarbonylamino group.

The sulfonamide group represented by each of R¹⁰, R¹¹, R¹², R¹³, R¹⁴, R¹⁵, R¹⁶, and R¹⁷ is, for example, preferably a methanesulfonamide group, an ethanesulfonamide group, a trifluoromethanesulfonamide group, a benzenesulfonamide group, or a 4-toluenesulfonamide group.

The sulfamoyl group represented by each of R¹⁰, R¹¹, R¹², R¹³, R¹⁴, R¹⁵, R¹⁶, and R¹⁷ may have a substituent or may not have a substituent.

The substituent that the sulfamoyl group represented by each of R¹⁰, R¹¹, R¹², R¹³, R¹⁴, R¹⁵, R¹⁶, and R¹⁷ may have is, for example, preferably an alkyl group.

The sulfamoyl group represented by each of R¹⁰, R¹¹, R¹², R¹³, R¹⁴, R¹⁵, R¹⁶, and R¹⁷ is, for example, preferably a methylsulfamoyl group or a dimethylsulfamoyl group.

The alkylthio group represented by each of R¹⁰, R¹¹, R¹², R¹³, R¹⁴, R¹⁵, R¹⁶, and R¹⁷ is, for example, preferably an alkylthio group whose alkyl moiety has 1 to 30 carbon atoms, suitable examples of which include a methylthio group and an ethylthio group.

The arylthio group represented by each of R¹⁰, R¹¹, R¹², R¹³, R¹⁴, R¹⁵, R¹⁶, and R¹⁷ is, for example, preferably an arylthio group whose aryl moiety has 6 to 30 carbon atoms, suitable examples of which include a phenylthio group and a 4-methylthio group.

In Formula (1), R¹⁰, R¹¹, R¹², R¹³, R¹⁴, R¹⁵, R¹⁶, and R¹⁷ are each particularly preferably a hydrogen atom, for example, from the viewpoint of light resistance.

In Formula (1), R²⁰ represents a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, an arylthio group, or a group represented by Formula (2).

In Formula (1), the details (for example, specific examples and preferred aspects) of the halogen atom, the alkyl group, the aryl group, the alkoxy group, the aryloxy group, the acyloxy group, the alkoxycarbonyl group, the carbamoyl group, the acyl group, the amino group, the amide group, the carbamoylamino group, the alkoxycarbonylamino group, the sulfonamide group, the sulfamoyl group, the alkylthio group, and the arylthio group represented by R²⁰ are the same as those shown for R¹⁰, R¹¹, R¹², R¹³, R¹⁴, R¹⁵, R¹⁶, and R¹⁷.

R²⁰ is preferably a hydrogen atom or a group represented by Formula (2), for example, from the viewpoint of light resistance.

In Formula (2), B represents a cationic aromatic hydrocarbon group, a cationic heterocyclic group, or a cationic heterocyclic methine group.

The cationic aromatic hydrocarbon group represented by B may have a substituent or may not have a substituent.

The substituent that the cationic aromatic hydrocarbon group represented by B may have is, for example, preferably a halogen atom, an alkyl group, an aryl group, an alkoxy group, an alkoxycarbonyl group, or an amino group.

Specific examples of the cationic aromatic hydrocarbon group represented by B include a cation of benzene, a cation of naphthalene, a cation of anthracene, a cation of phenanthrene, a cation of chrysene, a cation of pyrene, and a cation of azulene, each of those aromatic hydrocarbons may have a substituent. The cationic aromatic hydrocarbon group represented by B is, for example, particularly preferably a group represented by Formula (B-1) which will be described later or a group represented by Formula (B-2) which will be described later.

The cationic heterocyclic group represented by B may have a substituent or may not have a substituent.

The substituent that the cationic heterocyclic group represented by B may have is, for example, preferably a halogen atom, an alkyl group, an aryl group, an alkoxy group, or an alkoxycarbonyl group.

The cationic heterocyclic group represented by B is, for example, preferably a cation of pyrrole or a cation of imidazole, each of those heterocyclic compounds may have a substituent, and particularly preferably a group represented by Formula (B-3) which will be described later.

The cationic heterocyclic methine group represented by B may have a substituent or may not have a substituent.

The substituent that the cationic heterocyclic methine group represented by B may have is, for example, preferably a halogen atom, an alkyl group, an aryl group, an alkoxy group, or an alkoxycarbonyl group.

Specific examples of the cationic heterocyclic methine group represented by B include a cation of benzoxazol-2-ylidene methyl, a cation of benzothiazol-2-ylidene methyl, a cation of benzimidazol-2-ylidene methyl, a cation of indol-2-ylidene methyl, a cation of quinolin-2-ylidene methyl, and a cation of quinolin-4-ylidene methyl, each of those heterocyclic compounds may have a substituent.

The cationic heterocyclic methine group represented by B is, for example, particularly preferably a group represented by Formula (B-4) which will be described later or a group represented by Formula (B-5) which will be described later.

In Formula (2), B is preferably a group represented by Formula (B-1), a group represented by Formula (B-2), a group represented by Formula (B-3), a group represented by Formula (B-4), or a group represented by Formula (B-5), as described hereinbefore.

Above all, B is particularly preferably a group represented by Formula (B-2).

The compound according to the embodiment of the present disclosure in which B in Formula (2) is a group represented by Formula (B-2) tends to have maximum absorption in a near-infrared range on a longer wavelength side and exhibit more excellent light resistance, as compared with a case where B in Formula (2) is another group.

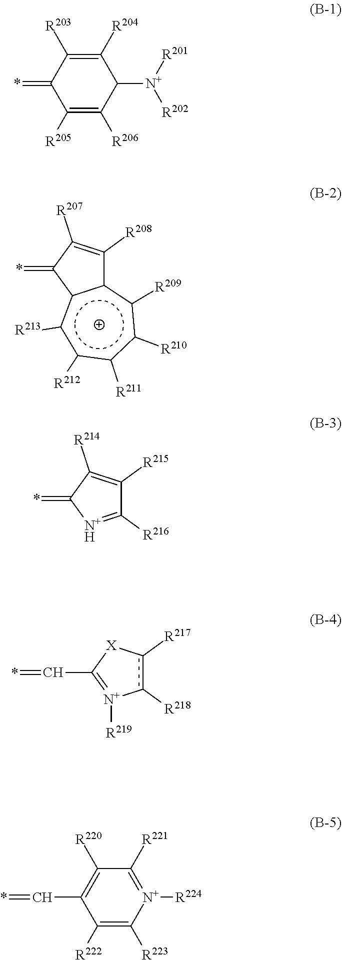

In Formula (B-1), Formula (B-2), Formula (B-3), Formula (B-4), and Formula (B-5), R²⁰¹, R²⁰², R²⁰³, R²⁰⁴, R²⁰⁵, R²⁰⁶, R²⁰⁷, R²⁰⁸, R²⁰⁹, R²¹⁰, R²¹¹, R²¹², R²¹³, R²¹⁴, R²¹⁵, R²¹⁶, R²¹⁷, R²¹⁸, R²¹⁹, R²²⁰, R²²¹, R²²², R²²³, and R²²⁴ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, X represents —O—, —S—, —CH═CH—, or —CR²²⁵R²²⁶— where R²²⁵ and R²²⁶ each independently represent an alkyl group, a bond between the carbon atom to which R²¹⁷ is bonded and the carbon atom to which R²¹⁸ is bonded represents a single bond or a double bond, * represents a bonding position, and R²⁰¹, R²⁰², R²⁰³, R²⁰⁴, R²⁰⁵, R²⁰⁶, R²⁰⁷, R²⁰⁸, R²⁰⁹, R²¹⁰, R²¹¹, R²¹², R²¹³, R²¹⁴, R²¹⁵, R²¹⁶, R²¹⁷, R²¹⁸, R²¹⁹, R²²⁰, R²²¹, R²²², R²²³, R²²⁴, R²²⁵, and R²²⁶ each may be bonded to an adjacent group to form a ring.

In Formula (B-1), Formula (B-2), Formula (B-3), Formula (B-4), and Formula (B-5), specific examples of the halogen atom, alkyl group, aryl group, alkoxy group, aryloxy group, acyloxy group, alkoxycarbonyl group, carbamoyl group, acyl group, amino group, amide group, carbamoylamino group, alkoxycarbonylamino group, sulfonamide group, sulfamoyl group, alkylthio group, and arylthio group represented by each of R²⁰¹, R²⁰², R²⁰³, R²⁰⁴, R²⁰⁵, R²⁰⁶, R²⁰⁷, R²⁰⁸, R²⁰⁹, R²¹⁰, R²¹¹, R²¹², R²¹³, R²¹⁴, R²¹⁵, R²¹⁶, R²¹⁷, R²¹⁸, R²¹⁹, R²²⁰, R²²¹, R²²², R²²³, and R²²⁴ are the same as those shown for R¹⁰, R¹¹, R¹², R¹³, R¹⁴, R¹⁵, R¹⁶, and R¹⁷.

In Formula (B-1), R²⁰¹, R²⁰², R²⁰³, R²⁰⁴, R²⁰⁵, and R²⁰⁶ each independently represent a hydrogen atom, a halogen atom, a nitro group, an alkyl group, an aryl group, a hydroxy group, alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group.

R²⁰¹ and R²⁰² are each independently preferably a hydrogen atom, an alkyl group, or an aryl group.

The alkyl group represented by each of R²⁰¹ and R²⁰² may have a substituent or may not have a substituent.

The alkyl group represented by each of R²⁰¹ and R²⁰² may be a linear alkyl group, an alkyl group having a branch, or an alkyl group having a cyclic structure.

The alkyl group represented by each of R²⁰¹ and R²⁰² is preferably an alkyl group having 1 to 30 carbon atoms.

The alkyl group represented by each of R²⁰¹ and R²⁰² is, for example, preferably a methyl group, an ethyl group, an n-propyl group, an isopropyl group, an n-octyl group, an eicosyl group, a benzyl group, a 2-ethylhexyl group, an allyl group, a prenyl group, a geranyl group, an oleyl group, a propargyl group, a cyclohexyl group, a cyclopentyl group, or a 2-hydroxyethyl group.

R²⁰¹ and R²⁰² may be bonded to each other to form a pyrrolidine ring or a piperidine ring.

The aryl group represented by each of R²⁰¹ and R²⁰² may have a substituent or may not have a substituent.

The substituent that the aryl group represented by each of R²⁰¹ and R²⁰² may have is, for example, preferably a halogen atom, an alkyl group, or an aryl group.

The aryl group represented by each of R²⁰¹ and R²⁰² is preferably an aryl group having 6 to 30 carbon atoms.

The aryl group represented by each of R²⁰¹ and R²⁰² is, for example, preferably a phenyl group, a p-tolyl group, a naphthyl group, or an m-chlorophenyl group.

R²⁰³, R²⁰⁴, R²⁰⁵, and R²⁰⁶ are each independently preferably a hydrogen atom, an alkyl group (for example, a methyl group), an aryl group (for example, a phenyl group), an amide group (for example, an acetamide group or a pivaloylamino group), a hydroxy group, or an alkoxy group (for example, a methoxy group).

In Formula (B-2), R²⁰⁷, R²⁰⁸, R²⁰⁹, R²¹⁰, R²¹¹, R²¹², and R²¹³ each independently represent a hydrogen atom, a halogen atom, a nitro group, an alkyl group, an aryl group, a hydroxy group, alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, among which a hydrogen atom or an alkyl group (for example, a methyl group, an ethyl group, or an iso-propyl group) is preferable.

In Formula (B-3), R²¹⁴, R²¹⁵, and R²¹⁶ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, among which a hydrogen atom or an alkyl group (for example, a methyl group or an ethyl group) is preferable.

In Formula (B-4), R²¹⁷, R²¹⁸, and R²¹⁹ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group.

R²¹⁷ and R²¹⁸ are each independently preferably a hydrogen atom or an alkyl group (for example, a methyl group). In addition, it is also preferable that R²¹⁷ and R²¹⁸ are linked to each other to form a ring (for example, a benzene ring or a naphthalene ring).

R²¹⁹ is preferably an alkyl group.

The alkyl group represented by R²¹⁹ may have a substituent or may not have a substituent.

The alkyl group represented by R²¹⁹ may be a linear alkyl group, an alkyl group having a branch, or an alkyl group having a cyclic structure.

The alkyl group represented by R²¹⁹ is preferably an alkyl group having 1 to 30 carbon atoms.

The alkyl group represented by R²¹⁹ is, for example, preferably a methyl group, an ethyl group, an n-propyl group, an n-butyl group, an iso-butyl group, an n-amyl group, an iso-amyl group, an n-octyl group, an eicosyl group, a benzyl group, a 2-ethylhexyl group, an allyl group, a prenyl group, a geranyl group, an oleyl group, a propargyl group, a cyclohexyl group, a cyclopentyl group, or a 2-hydroxyethyl group.

In Formula (B-4), X represents —O—, —S—, —CH═CH—, or —CR¹²⁵R¹²⁶— where R¹²⁵ and R¹²⁶ each independently represent an alkyl group.

R²²⁵ and R²²⁶ may be bonded to each other to form a ring.

X is preferably —O—, —S—, or —CH═CH—.

—CR¹²⁵R¹²⁶— represented by X is, for example, preferably dimethylmethylene, cyclohexylidene, or dibenzylmethylene.

In Formula (B-5), R²²⁰, R²²¹, R²²², R²²³, and R²²⁴ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group.

R²²⁰, R²²¹, R²²², and R²²³ are each independently preferably a hydrogen atom, an alkyl group (for example, a methyl group), an aryl group (for example, a phenyl group), a hydroxy group, or an alkoxy group (for example, a methoxy group).

R²²⁴ is preferably an alkyl group.

The alkyl group represented by R²²⁴ may be a linear alkyl group, an alkyl group having a branch, or an alkyl group having a cyclic structure.

The alkyl group represented by R²²⁴ is preferably an alkyl group having 1 to 30 carbon atoms.

The alkyl group represented by R²²⁴ is, for example, preferably a methyl group, an ethyl group, an n-propyl group, an n-butyl group, an iso-butyl group, an n-amyl group, an iso-amyl group, an n-octyl group, an eicosyl group, a benzyl group, a 2-ethylhexyl group, an allyl group, a prenyl group, a geranyl group, an oleyl group, a propargyl group, a cyclohexyl group, a cyclopentyl group, or a 2-hydroxyethyl group.

In Formula (1), R¹⁰, R¹¹, R¹², R¹³, R¹⁴, R¹⁵, R¹⁶, R¹⁷, and R²⁰ each may be bonded to an adjacent group to form a ring.

The ring to be formed may be a saturated ring or an unsaturated ring.

The ring to be formed is, for example, preferably a benzene ring or a naphthalene ring.

In Formula (1), A represents an aromatic hydrocarbon group, a heterocyclic group, or a heterocyclic methine group.

The aromatic hydrocarbon group represented by A may have a substituent or may not have a substituent.

The substituent that the aromatic hydrocarbon group represented by A may have is, for example, preferably a halogen atom, an alkyl group, an aryl group, an alkoxy group, an alkoxycarbonyl group, or an amino group.

Specific examples of the aromatic hydrocarbon group represented by A include benzene, naphthalene, anthracene, phenanthrene, chrysene, pyrene, and azulene, each of which may have a substituent. The aromatic hydrocarbon group represented by A is, for example, particularly preferably a group represented by Formula (A-1) which will be described later or a group represented by Formula (A-2) which will be described later.

The heterocyclic group represented by A may have a substituent or may not have a substituent.

The substituent that the heterocyclic group represented by A may have is, for example, preferably a halogen atom, an alkyl group, an aryl group, an alkoxy group, or an alkoxycarbonyl group.

The heterocyclic group represented by A is, for example, preferably pyrrole or imidazole, each of which may have a substituent, and particularly preferably a group represented by Formula (A-3) which will be described later.

The heterocyclic methine group represented by A may have a substituent or may not have a substituent.

The substituent that the heterocyclic methine group represented by A may have is, for example, preferably a halogen atom, an alkyl group, an aryl group, an alkoxy group, or an alkoxycarbonyl group.

Specific examples of the heterocyclic methine group represented by A include benzoxazol-2-ylidene methyl, benzothiazol-2-ylidene methyl, benzimidazol-2-ylidene methyl, indol-2-ylidene methyl, quinolin-2-ylidene methyl, and quinolin-4-ylidene methyl, each of which may have a substituent.

The heterocyclic methine group represented by A is, for example, particularly preferably a group represented by Formula (A-4) which will be described later or a group represented by Formula (A-5) which will be described later.

In Formula (1), A is preferably a group represented by Formula (A-1), a group represented by Formula (A-2), a group represented by Formula (A-3), a group represented by Formula (A-4), or a group represented by Formula (A-5), as described hereinbefore.

Above all, A is particularly preferably a group represented by Formula (A-2).

The compound according to the embodiment of the present disclosure in which A in Formula (1) is a group represented by Formula (A-2) tends to have maximum absorption in a near-infrared range on a longer wavelength side and exhibit more excellent light resistance, as compared with a case where A in Formula (1) is another group.

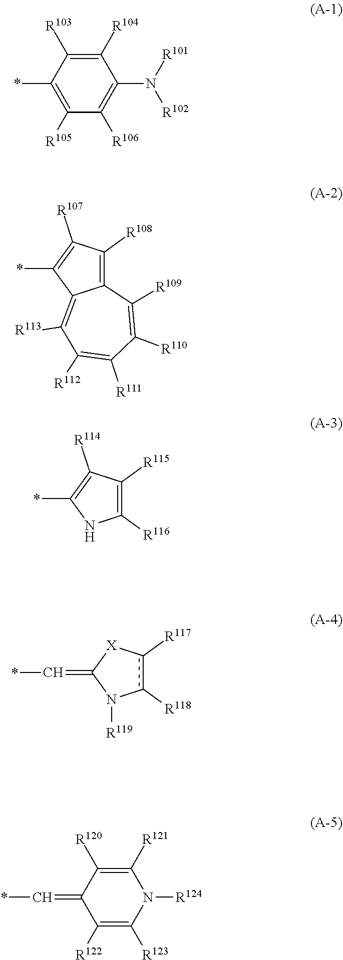

In Formula (A-1), Formula (A-2), Formula (A-3), Formula (A-4), and Formula (A-5), R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹, R¹¹², R¹¹³, R¹¹⁴, R¹¹⁵, R¹¹⁶, R¹¹⁷, R¹¹⁸, R¹¹⁹, R¹²⁰, R¹²¹, R¹²², R¹²³, and R¹²⁴ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, X represents —O—, —S—, —CH═CH—, or —CR¹²⁵R¹²⁶— where R¹²⁵ and R¹²⁶ each independently represent an alkyl group, a bond between the carbon atom to which R¹¹⁷ is bonded and the carbon atom to which R¹¹⁸ is bonded represents a single bond or a double bond, * represents a bonding position, and R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹, R¹¹², R¹¹³, R¹¹⁴, R¹¹⁵, R¹¹⁶, R¹¹⁷, R¹¹⁸, R¹¹⁹, R¹²⁰, R¹²¹, R¹²², R¹²³, R¹²⁴, R¹²⁵, and R¹²⁶ each may be bonded to an adjacent group to form a ring.

In Formula (A-1), Formula (A-2), Formula (A-3), Formula (A-4), and Formula (A-5), specific examples of the halogen atom, alkyl group, aryl group, alkoxy group, aryloxy group, acyloxy group, alkoxycarbonyl group, carbamoyl group, acyl group, amino group, amide group, carbamoylamino group, alkoxycarbonylamino group, sulfonamide group, sulfamoyl group, alkylthio group, and arylthio group represented by each of R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹, R¹¹², R¹¹³, R¹¹⁴, R¹¹⁵, R¹¹⁶, R¹¹⁷, R¹¹⁸, R¹¹⁹, R¹²⁰, R¹²¹, R¹²², R¹²³, and R¹²⁴ are the same as those shown for R¹⁰, R¹¹, R¹², R¹³, R¹⁴, R¹⁵, R¹⁶, and R¹⁷.

In Formula (A-1), R¹⁰¹, R¹⁰², R¹⁰³, R¹⁰⁴, R¹⁰⁵, and R¹⁰⁶ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group.

R¹⁰¹ and R¹⁰² are each independently preferably a hydrogen atom, an alkyl group, or an aryl group.

The alkyl group represented by each of R¹⁰¹ and R¹⁰² may have a substituent or may not have a substituent.

The alkyl group represented by each of R¹⁰¹ and R¹⁰² may be a linear alkyl group, an alkyl group having a branch, or an alkyl group having a cyclic structure.

The alkyl group represented by each of R¹⁰¹ and R¹⁰² is preferably an alkyl group having 1 to 30 carbon atoms.

The alkyl group represented by each of R¹⁰¹ and R¹⁰² is, for example, preferably a methyl group, an ethyl group, an n-propyl group, an isopropyl group, an n-octyl group, an eicosyl group, a benzyl group, a 2-ethylhexyl group, an allyl group, a prenyl group, a geranyl group, an oleyl group, a propargyl group, a cyclohexyl group, a cyclopentyl group, or a 2-hydroxyethyl group.

R¹⁰¹ and R¹⁰² may be bonded to each other to form a pyrrolidine ring or a piperidine ring.

The aryl group represented by each of R¹⁰¹ and R¹⁰² may have a substituent or may not have a substituent.

The substituent that the aryl group represented by each of R¹⁰¹ and R¹⁰² may have is, for example, preferably a halogen atom, an alkyl group, or an aryl group.

The aryl group represented by each of R¹⁰¹ and R¹⁰² is preferably an aryl group having 6 to 30 carbon atoms.

The aryl group represented by each of R¹⁰¹ and R¹⁰² is, for example, preferably a phenyl group, a p-tolyl group, a naphthyl group, or an m-chlorophenyl group.

R¹⁰³, R¹⁰⁴, R¹⁰⁵, and R¹⁰⁶ are each independently preferably a hydrogen atom, an alkyl group (for example, a methyl group), an aryl group (for example, a phenyl group), an amide group (for example, an acetamide group or a pivaloylamino group), a hydroxy group, or an alkoxy group (for example, a methoxy group).

In Formula (A-2), R¹⁰⁷, R¹⁰⁸, R¹⁰⁹, R¹¹⁰, R¹¹¹, R¹¹², and R¹¹³ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, among which a hydrogen atom or an alkyl group (for example, a methyl group, an ethyl group, or an iso-propyl group) is preferable.

In Formula (A-3), R¹¹⁴, R¹¹⁵, and R¹¹⁶ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, among which a hydrogen atom or an alkyl group (for example, a methyl group or an ethyl group) is preferable.

In Formula (A-4), R¹¹⁷, R¹¹⁸, and R¹¹⁹ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group.

R¹¹⁷ and R¹¹⁸ are each independently preferably a hydrogen atom or an alkyl group (for example, a methyl group). In addition, it is also preferable that R¹¹⁷ and R¹¹⁸ are linked to each other to form a ring (for example, a benzene ring or a naphthalene ring).

R¹¹⁹ is preferably an alkyl group.

The alkyl group represented by R¹¹⁹ may have a substituent or may not have a substituent.

The alkyl group represented by R¹¹⁹ may be a linear alkyl group, an alkyl group having a branch, or an alkyl group having a cyclic structure.

The alkyl group represented by R¹¹⁹ is preferably an alkyl group having 1 to 30 carbon atoms.

The alkyl group represented by R¹¹⁹ is, for example, preferably a methyl group, an ethyl group, an n-propyl group, an n-butyl group, an iso-butyl group, an n-amyl group, an iso-amyl group, an n-octyl group, an eicosyl group, a benzyl group, a 2-ethylhexyl group, an allyl group, a prenyl group, a geranyl group, an oleyl group, a propargyl group, a cyclohexyl group, a cyclopentyl group, or a 2-hydroxyethyl group.

In Formula (A-4), X represents —O—, —S—, —CH═CH—, or —CR¹²⁵R¹²⁶— where R¹²⁵ and R¹²⁶ each independently represent an alkyl group.

R¹²⁵ and R¹²⁶ may be bonded to each other to form a ring.

X is preferably —O—, —S—, or —CH═CH—.

—CR¹²⁵R¹²⁶— represented by X is, for example, preferably dimethylmethylene, cyclohexylidene, or dibenzylmethylene.

In Formula (A-5), R¹²⁰, R¹²¹, R¹²², R¹²³, and R¹²⁴ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group.

R¹²⁰, R¹²¹, R¹²², and R¹²³ are each independently preferably a hydrogen atom, an alkyl group (for example, a methyl group), an aryl group (for example, a phenyl group), a hydroxy group, or an alkoxy group (for example, a methoxy group).

R¹²⁴ is preferably an alkyl group.

The alkyl group represented by R¹²⁴ may be a linear alkyl group, an alkyl group having a branch, or an alkyl group having a cyclic structure.

The alkyl group represented by R¹²⁴ is preferably an alkyl group having 1 to 30 carbon atoms.

The alkyl group represented by R¹²⁴ is, for example, preferably a methyl group, an ethyl group, an n-propyl group, an n-butyl group, an iso-butyl group, an n-amyl group, an iso-amyl group, an n-octyl group, an eicosyl group, a benzyl group, a 2-ethylhexyl group, an allyl group, a prenyl group, a geranyl group, an oleyl group, a propargyl group, a cyclohexyl group, a cyclopentyl group, or a 2-hydroxyethyl group.

Specific examples of the compound represented by Formula (1) will be shown below, but the present disclosure is not limited to these examples. It should be noted that “Me” represents a methyl group, “Et” represents an ethyl group, and “Ph” represents a phenyl group (the same applies hereinafter).

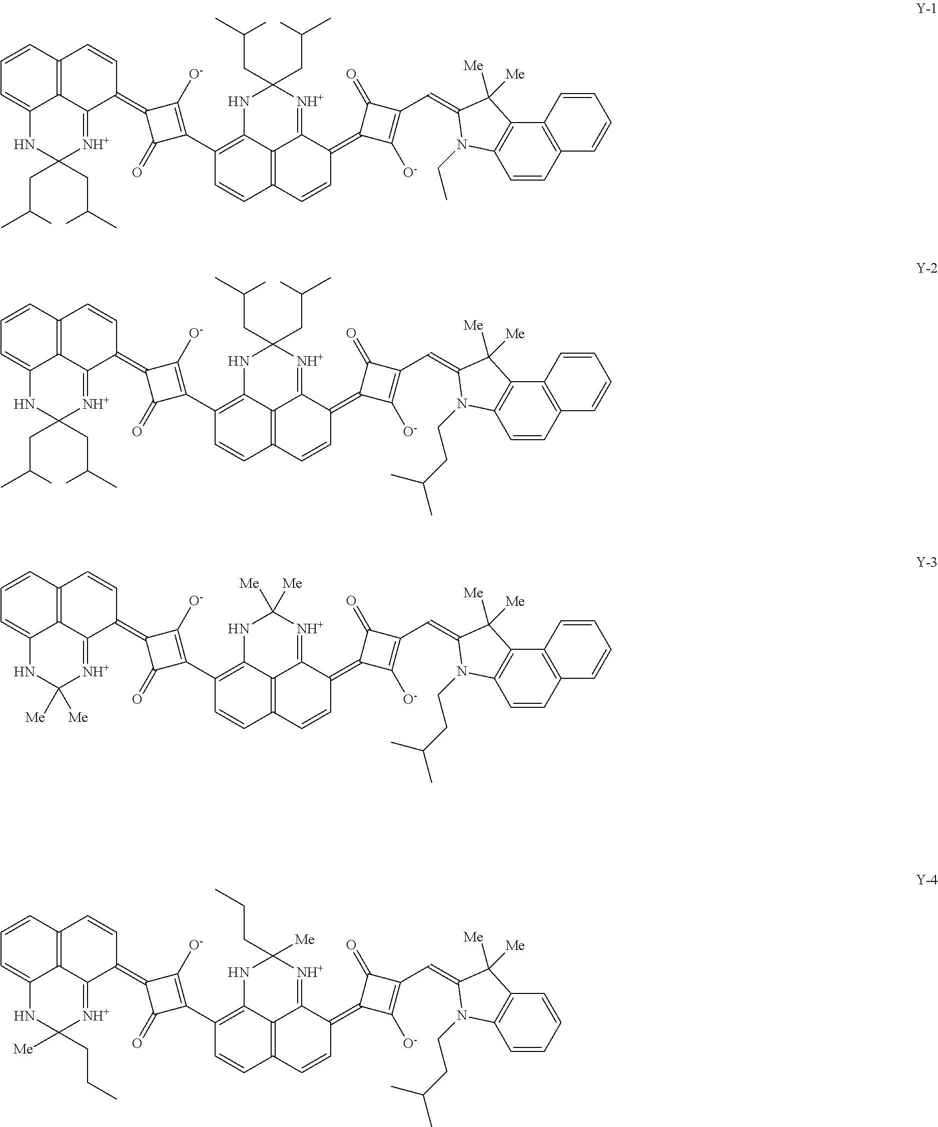

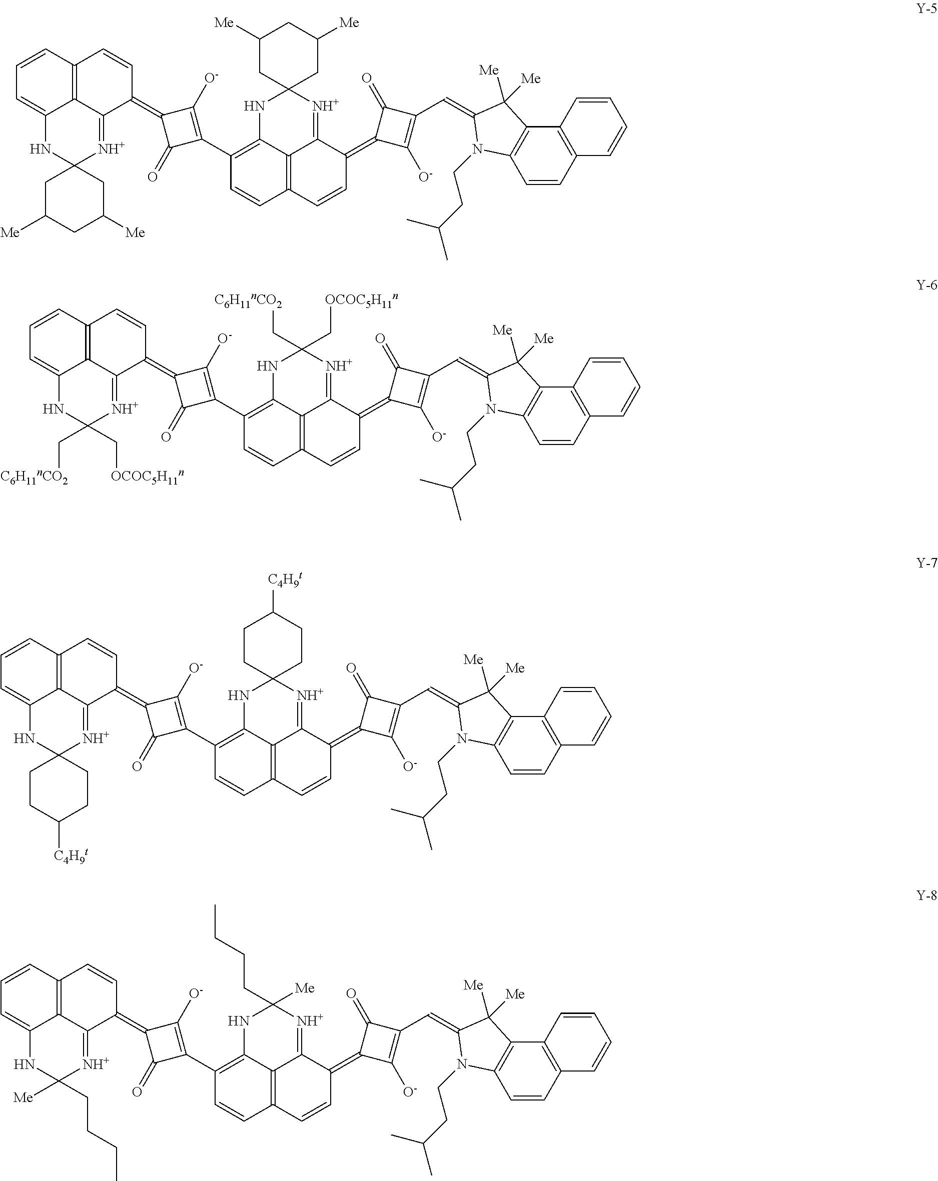

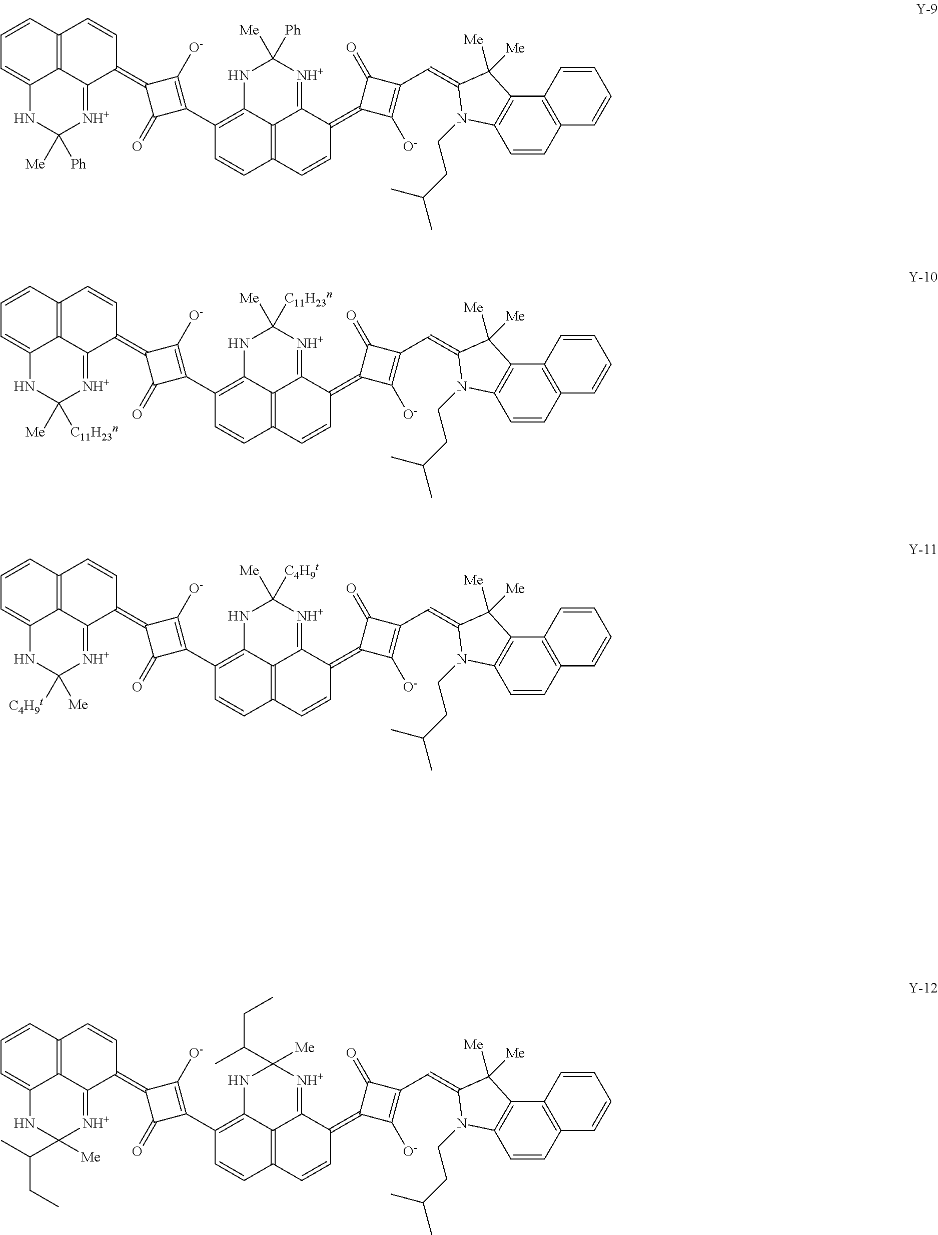

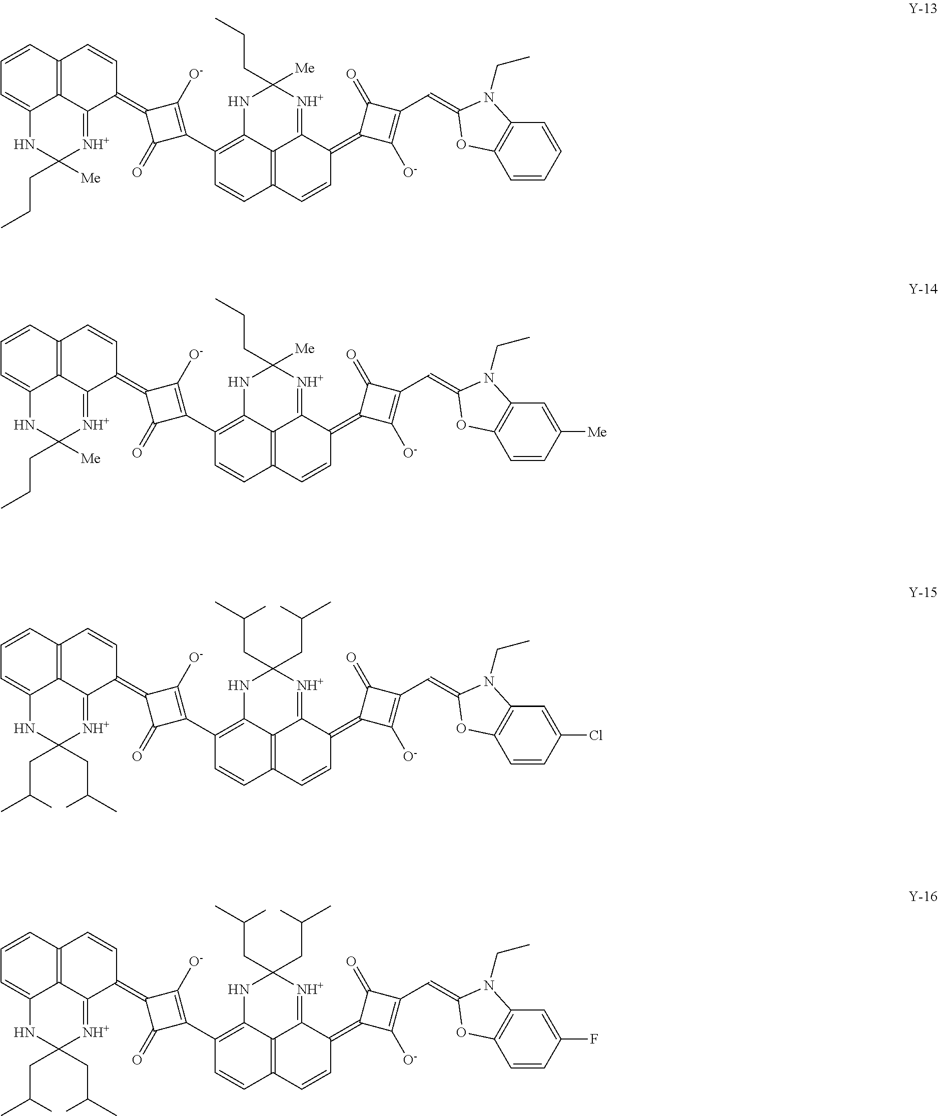

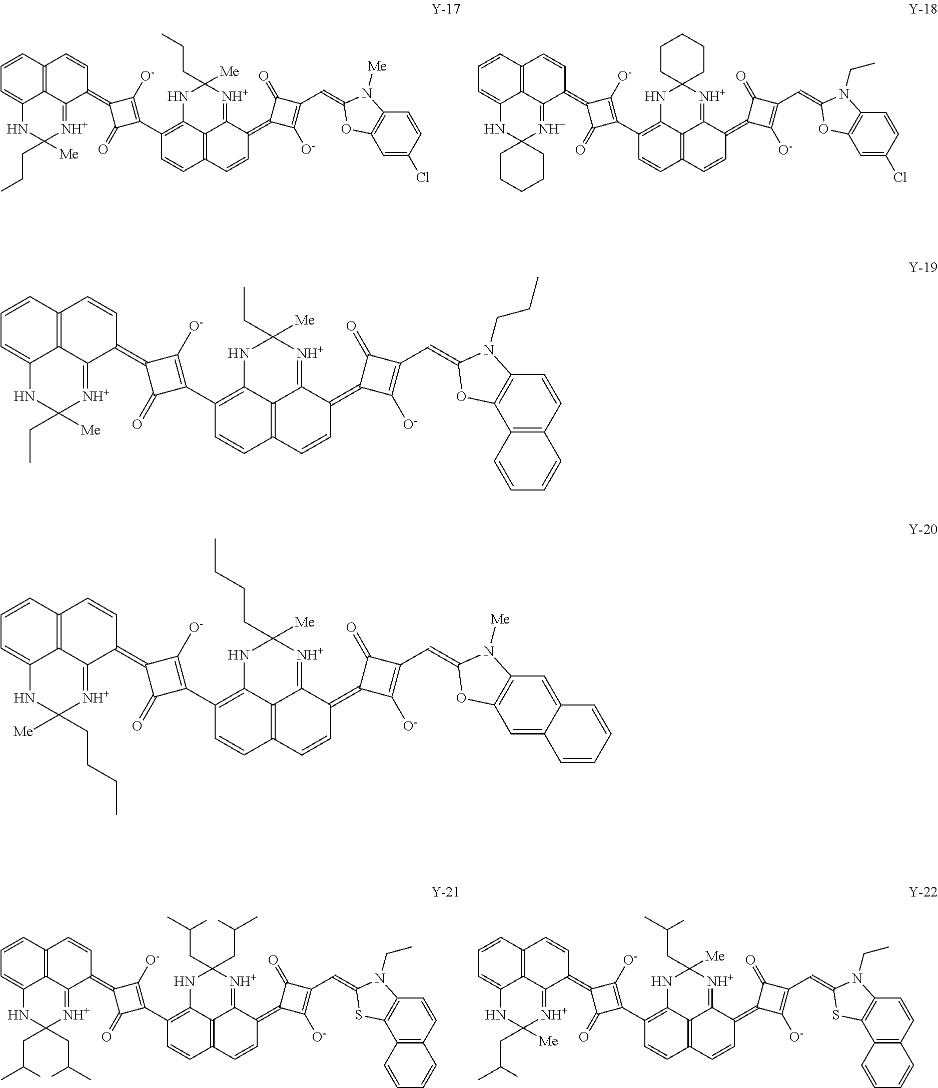

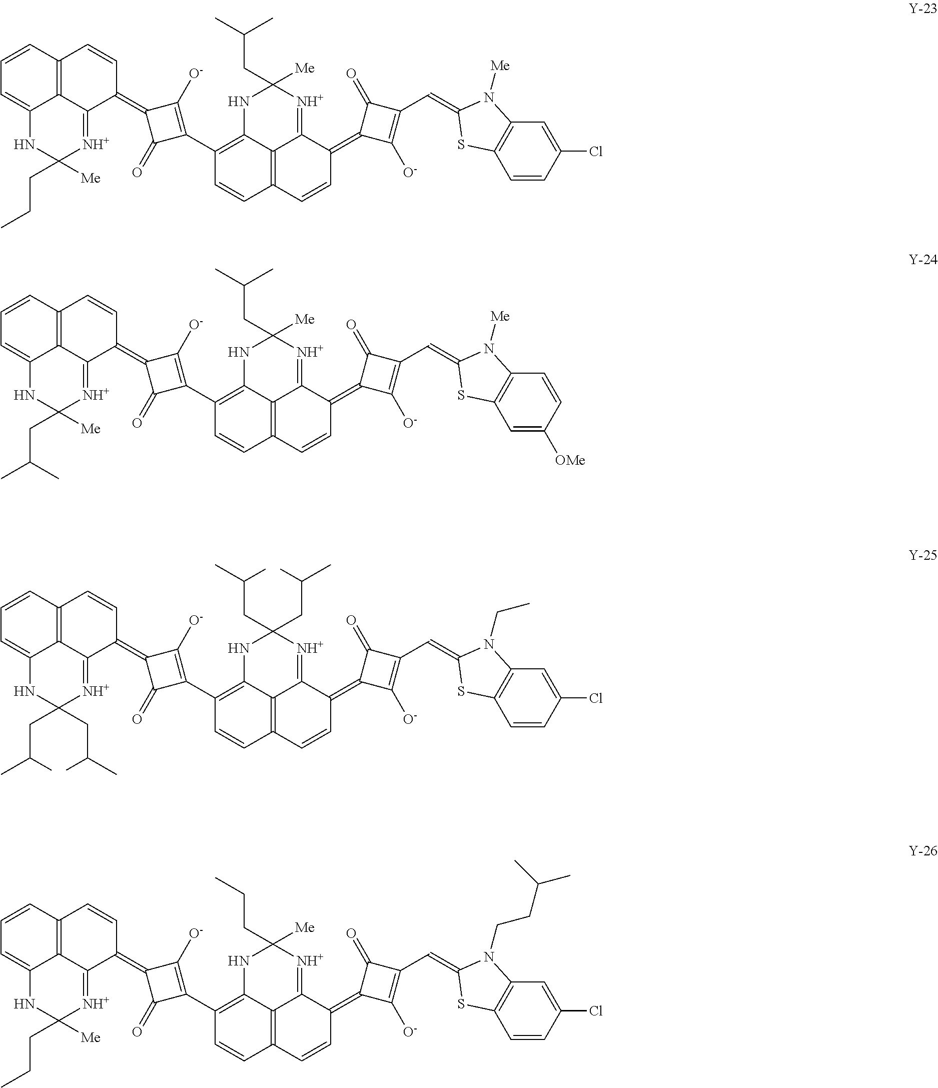

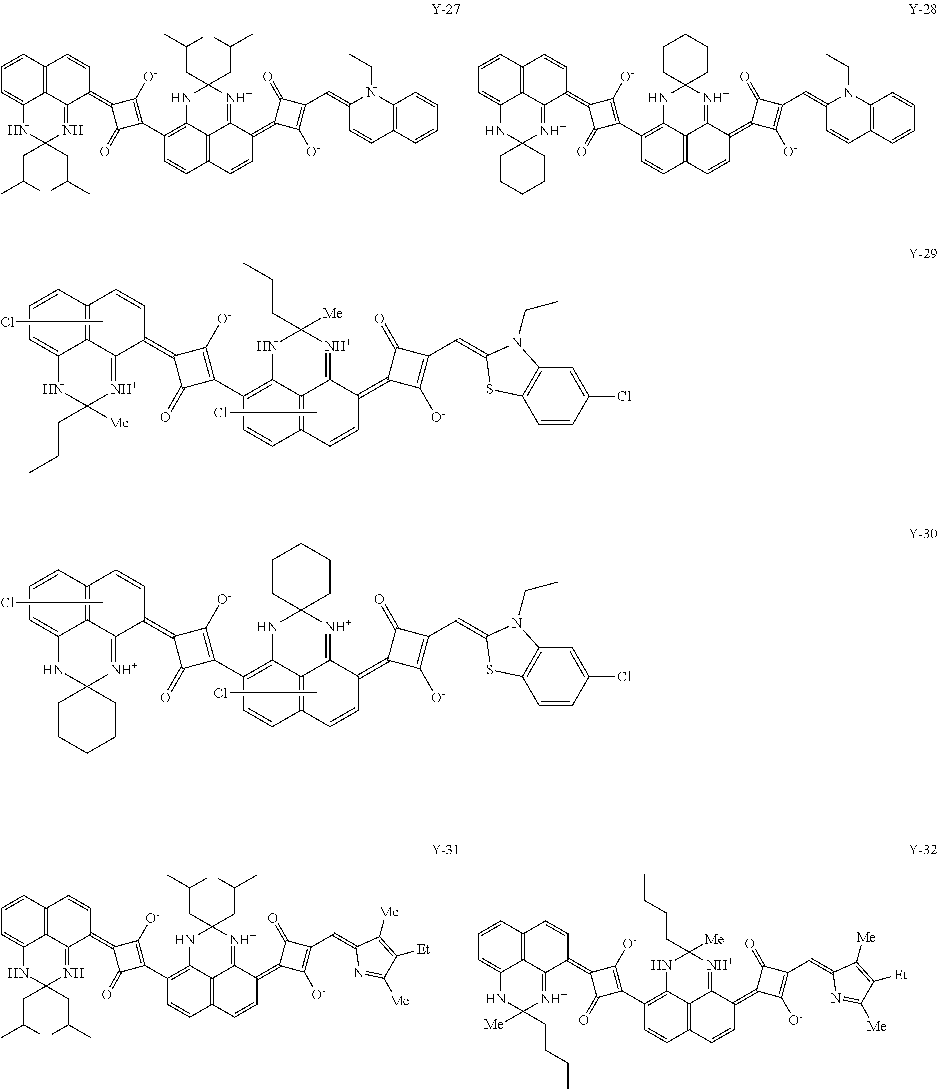

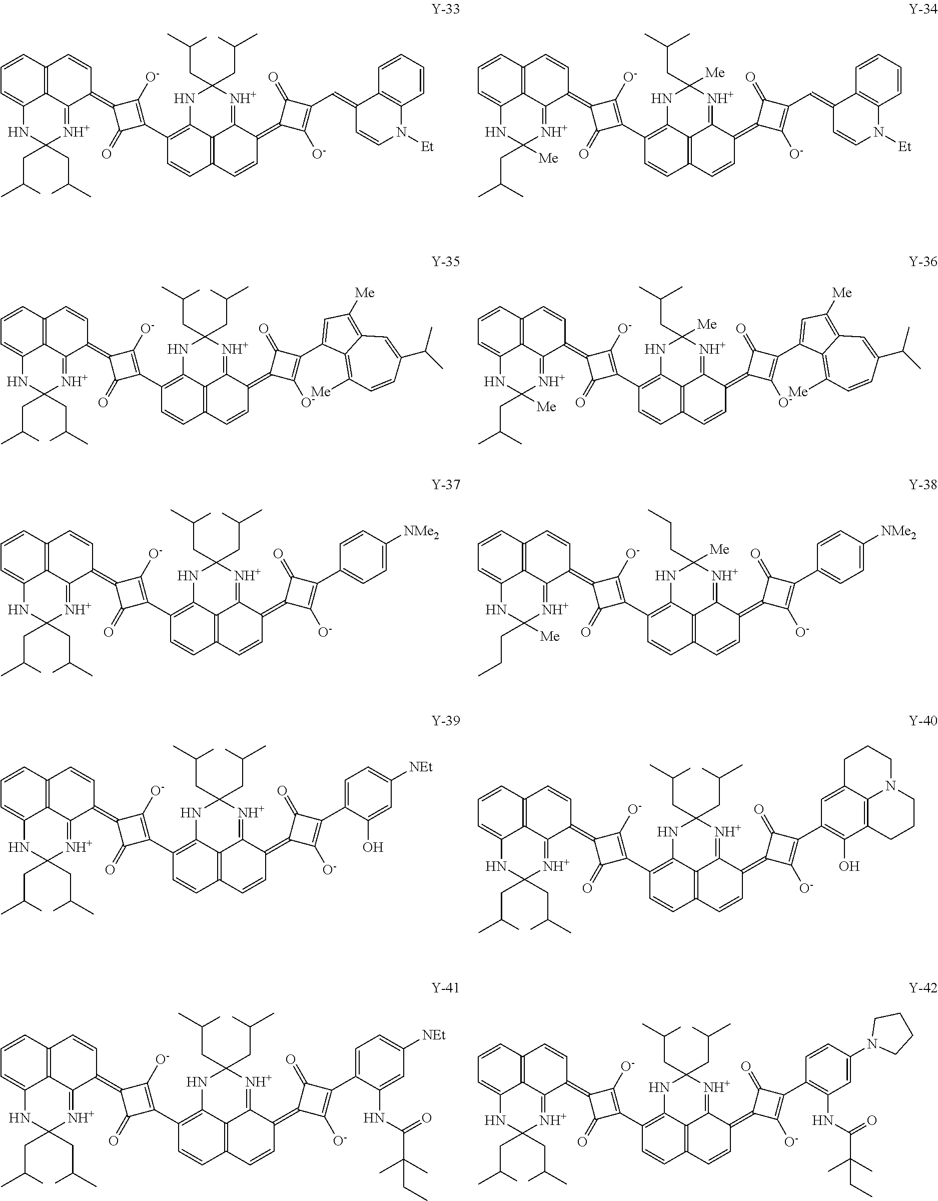

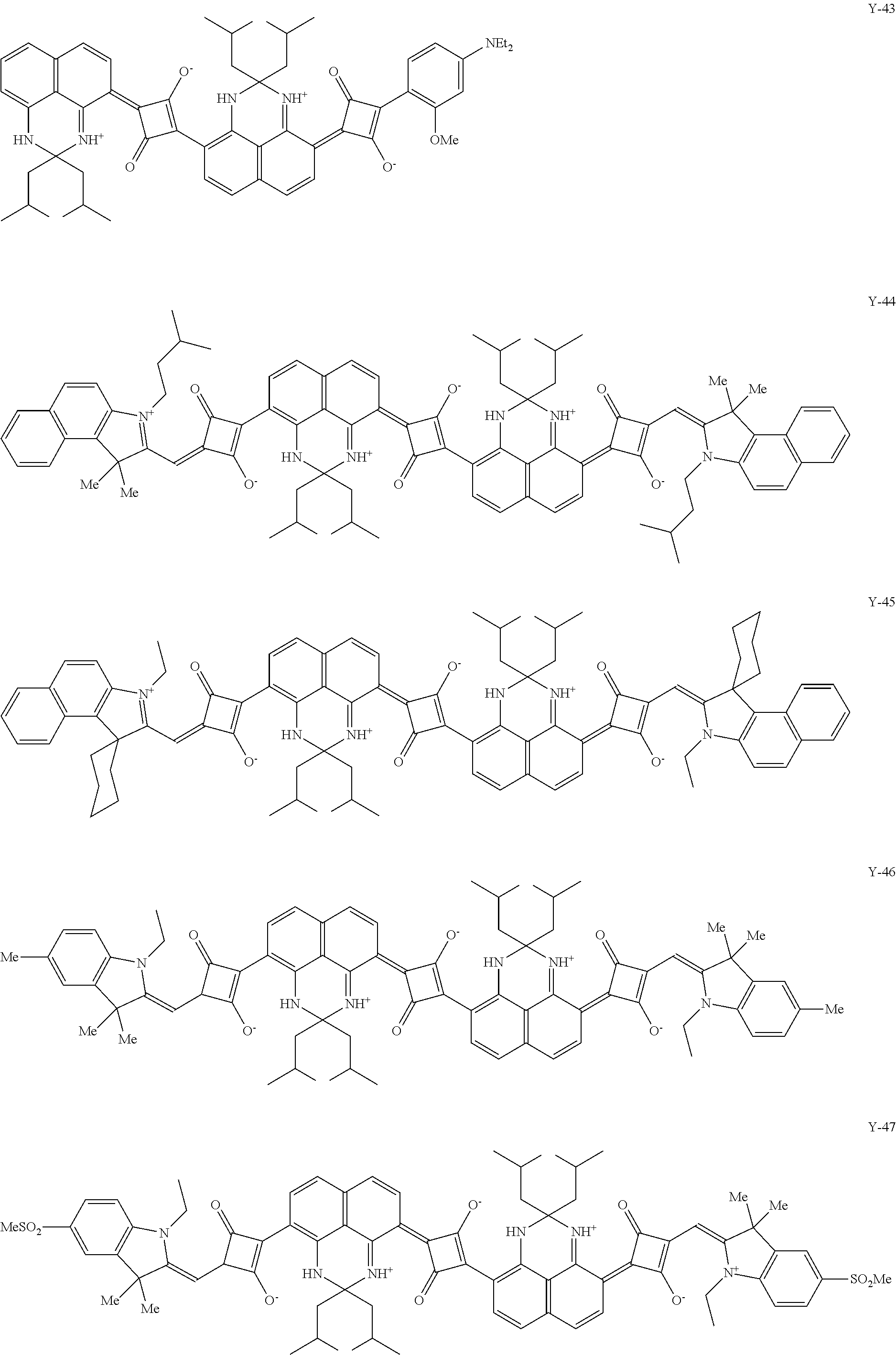

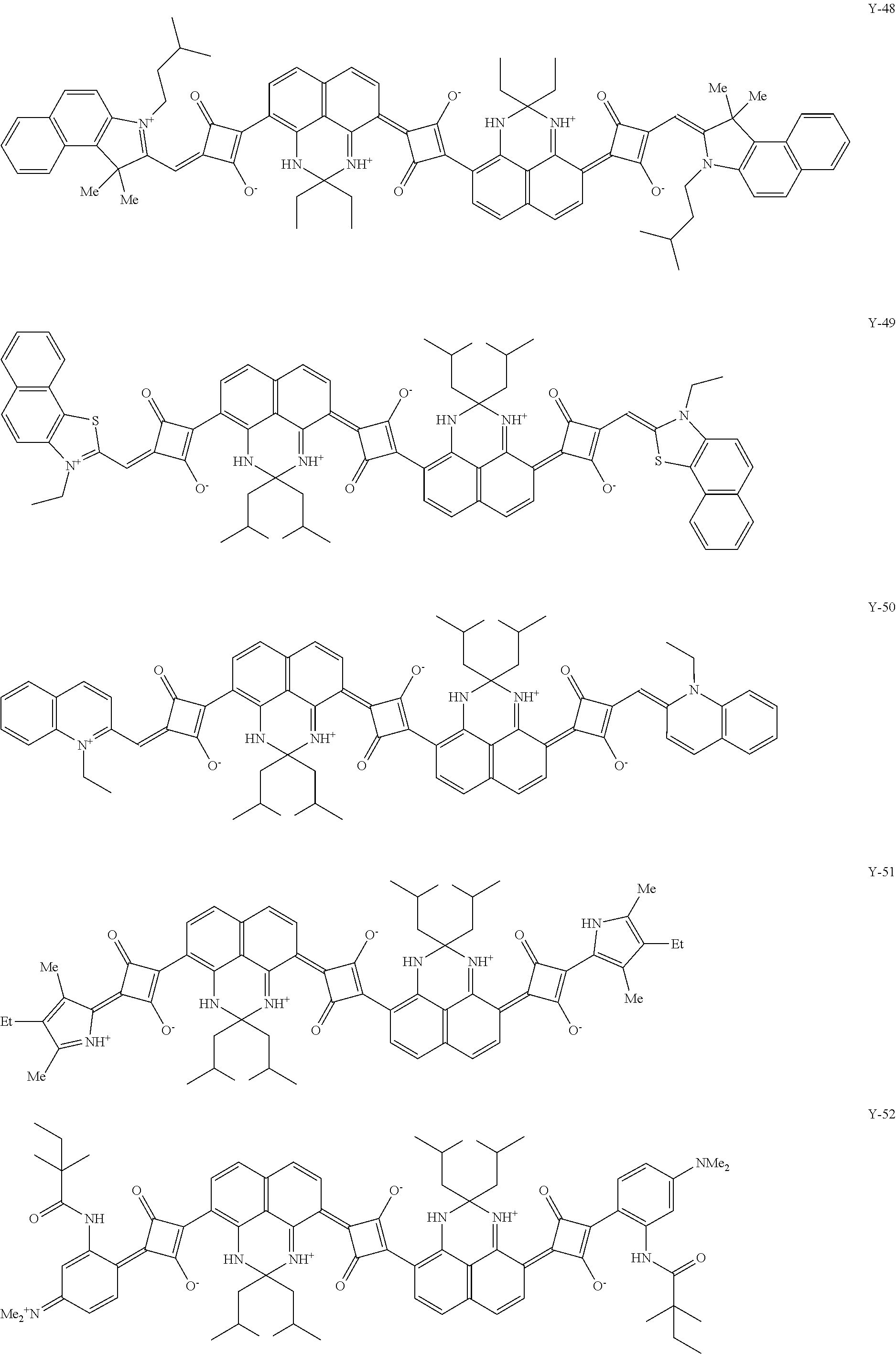

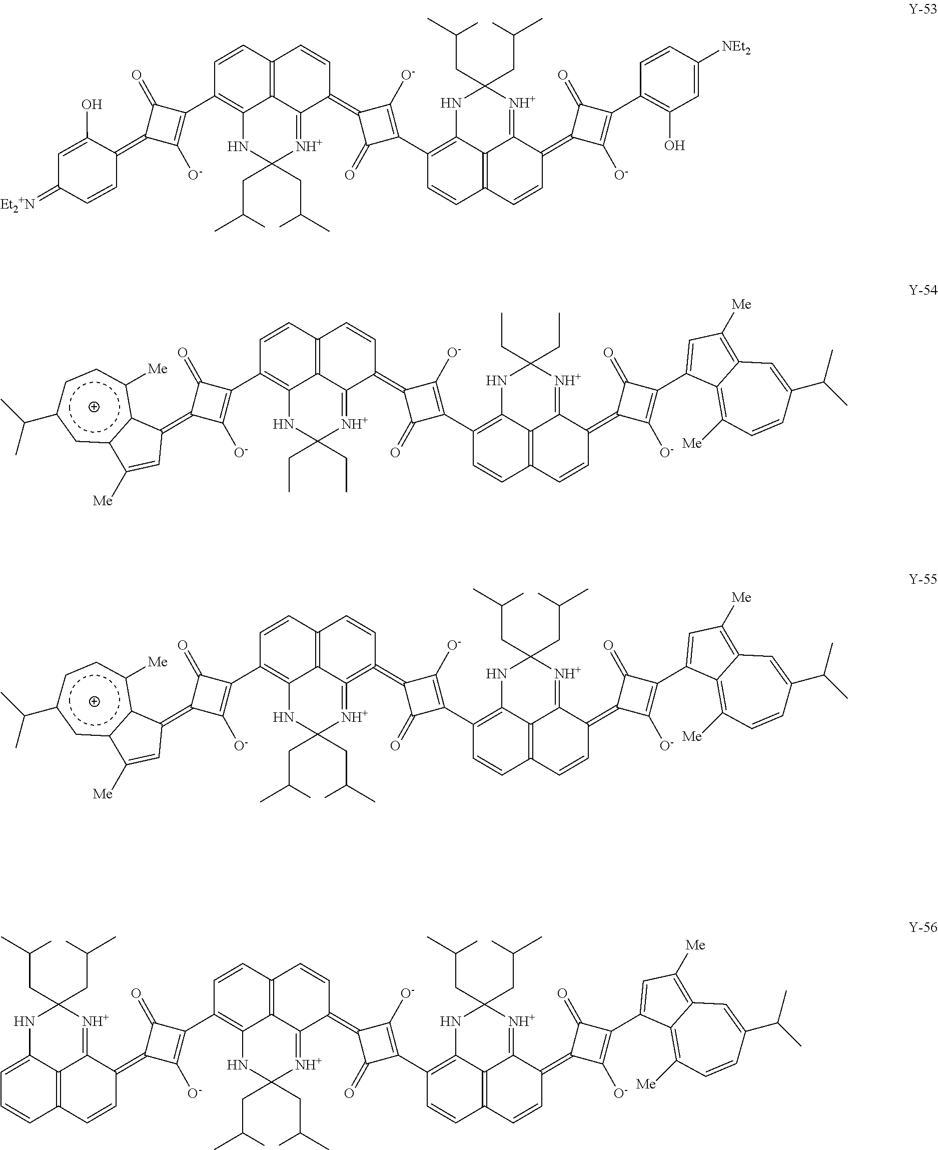

The compound represented by Formula (1) and the tautomer thereof have maximum absorption in a near-infrared range on a longer wavelength side than 850 nm.

The maximal absorption wavelength of the compound represented by Formula (1) and the tautomer thereof is preferably within a wavelength range of more than 850 nm to 1200 nm, and more preferably within a wavelength range of 900 nm to 1200 nm.

The molar absorption coefficient at the maximal absorption wavelength of the compound represented by Formula (1) and the tautomer thereof is preferably 100,000 L/(mol·cm) or more, more preferably 120,000 L/(mol·cm) or more, and still more preferably 150,000 L/(mol·cm) or more.

The maximal absorption wavelength and the molar absorption coefficient are measured using a spectrophotometer. With regard to a specific measuring method, reference can be made to the measuring method described in Examples which will be given later.

The molecular weight of the compound represented by Formula (1) and the tautomer thereof is not particularly limited and is, for example, preferably 500 to 3000 and more preferably 700 to 2000.

### [Method for Producing Compound Represented by Formula (1) or Tautomer Thereof]

The method for producing the compound represented by Formula (1) or the tautomer thereof (that is, the compound according to the embodiment of the present disclosure) (hereinafter, also referred to as a “production method according to the embodiment of the present disclosure”) is not particularly limited.

Although the compound according to the embodiment of the present disclosure can be produced by a known method or with reference to a known method, the compound can be more suitably produced by the production method according to the embodiment of the present disclosure described below. That is, as shown below, the compound according to the embodiment of the present disclosure can be produced by a simple method as compared with the related art's method for producing a compound having two or more squarylium skeletons.

The production method according to the embodiment of the present disclosure includes reacting a compound represented by Formula (3) with a compound represented by Formula (4).

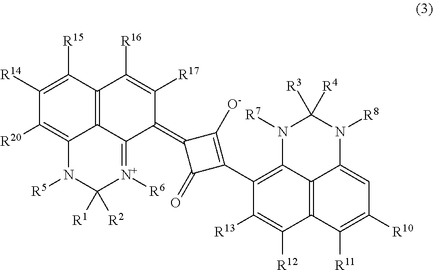

In Formula (3), R¹, R², R³, and R⁴ each independently represent a hydrogen atom, an alkyl group, or an aryl group.

R¹, R², R³, and R⁴ in Formula (3) each have the same definition as R¹, R², R³, and R⁴ in Formula (1), and the same applies to preferred aspects thereof, so the description thereof will not be repeated here.

In Formula (3), R⁵, R⁶, R⁷, and R⁸ each independently represent a hydrogen atom, an alkyl group, or an aryl group.

R⁵, R⁶, R⁷, and R⁸ in Formula (3) each have the same definition as R⁵, R⁶, R⁷, and R⁸ in Formula (1), and the same applies to preferred aspects thereof, so the description thereof will not be repeated here.

In Formula (3), R¹⁰, R¹¹, R¹², R¹³, R¹⁴, R¹⁵, R¹⁶, and R¹⁷ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group.

R¹⁰, R¹¹, R¹², R¹³, R¹⁴, R¹⁵, R¹⁶, and R¹⁷ in Formula (3) each have the same definition as R¹⁰, R¹¹, R¹², R¹³, R¹⁴, R¹⁵, R¹⁶, and R¹⁷ in Formula (1), and the same applies to preferred aspects thereof, so the description thereof will not be repeated here.

In Formula (3), R²⁰ represents a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, an arylthio group, or a group represented by Formula (2).

R²⁰ in Formula (3) has the same definition as R²⁰ in Formula (1), and the same applies to preferred aspects thereof, so the description thereof will not be repeated here.

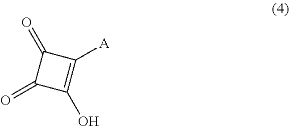

In Formula (4), A represents an aromatic hydrocarbon group, a heterocyclic group, or a heterocyclic methine group.

A in Formula (4) has the same definition as A in Formula (1), and the same applies to preferred aspects thereof, so the description thereof will not be repeated here.

In the production method according to the embodiment of the present disclosure in which the compound represented by Formula (3) is reacted with the compound represented by Formula (4), the following aspect 1, aspect 2, aspect 3, aspect 4, or aspect 5 is preferable, and aspect 2 is more preferable.

Aspect 1: an aspect in which A in Formula (4) is a group represented by Formula (A-1), R²⁰ in Formula (3) is a group represented by Formula (2), and B in Formula (2) is a group represented by Formula (B-1).

Aspect 2: an aspect in which A in Formula (4) is a group represented by Formula (A-2), R²⁰ in Formula (3) is a group represented by Formula (2), and B in Formula (2) is a group represented by Formula (B-2).

Aspect 3: an aspect in which A in Formula (4) is a group represented by Formula (A-3), R²⁰ in Formula (3) is a group represented by Formula (2), and B in Formula (2) is a group represented by Formula (B-3).

Aspect 4: an aspect in which A in Formula (4) is a group represented by Formula (A-4), R²⁰ in Formula (3) is a group represented by Formula (2), and B in Formula (2) is a group represented by Formula (B-4).

Aspect 5: an aspect in which A in Formula (4) is a group represented by Formula (A-5), R²⁰ in Formula (3) is a group represented by Formula (2), and B in Formula (2) is a group represented by Formula (B-5).

The reaction between the compound represented by Formula (3) and the compound represented by Formula (4) can be carried out in an organic solvent.

The organic solvent is not particularly limited and is, for example, preferably an aromatic hydrocarbon such as benzene, toluene, chlorobenzene, or xylene, an alcohol such as n-butanol, iso-butanol, or n-pentanol, or a mixed liquid thereof, and particularly preferably a mixed liquid of toluene and n-butanol.

The reaction is preferably carried out under refluxing of the organic solvent, while produced water is azeotropically dehydrated.

The reaction ratio between the compound represented by Formula (3) and the compound represented by Formula (4) can be appropriately set depending on the desired structure of the compound represented by Formula (1).

The reaction temperature is not particularly limited and is, for example, preferably 15° C. to a boiling point of a reaction solvent.

The reaction time is not particularly limited and can be set to, for example, 1 hour to 48 hours.

The production method according to the embodiment of the present disclosure can produce the compound represented by Formula (1) with a desired structure by controlling conditions such as reaction ratio and reaction time between the compound represented by Formula (3) and the compound represented by Formula (4), and is therefore excellent in manufacturing suitability.

### Uses

The use of the compound according to the embodiment of the present disclosure is not particularly limited.

Examples of the use of the compound according to the embodiment of the present disclosure include optical films [for example, an infrared cut film for a display or a solid-state imaging element (for example, CCD or CMOS) and a heat ray shielding film], photothermal conversion materials [for example, a write-once optical disc, a laser welding material, and a flash fusing material], image forming materials [for example, a security ink and an invisible bar code ink], medical materials [for example, a diagnostic marker and a drug for photodynamic therapy], lenses having a function of absorbing or cutting near-infrared rays [for example, a lens for a camera such as a digital camera, a mobile phone, or an in-vehicle camera, and an optical lens such as an F-theta lens or a pickup lens], agricultural coating agents for the selective use of sunlight, protective glasses, and sunglasses. In addition, the compound according to the embodiment of the present disclosure can also be used to prevent copying of confidential documents. Additionally, the compound according to the embodiment of the present disclosure can be used in a fiber having heat retention and heat storage properties, an eye strain prevention agent, or a marking agent for positioning a photograph or a film. In addition, the compound according to the embodiment of the present disclosure can also impart camouflage properties (so-called camouflage performance) to a fiber or the like against reconnaissance using infrared rays.

### Composition

The composition according to the embodiment of the present disclosure contains the compound represented by Formula (1) described above or the tautomer thereof (that is, the compound according to the embodiment of the present disclosure).

The composition according to the embodiment of the present disclosure contains the compound according to the embodiment of the present disclosure, and is therefore suppressed from coloring in a visible range, has maximum absorption in a near-infrared range on a longer wavelength side than 850 nm, and has excellent light resistance. Therefore, for example, in a case where the composition according to the embodiment of the present disclosure is used to form a film, it is possible to obtain a film which is suppressed from coloring in a visible range, has maximum absorption in a near-infrared range on a longer wavelength side than 850 nm, and has excellent light resistance. The composition according to the embodiment of the present disclosure is suitable as a near-infrared absorbing material.

The composition according to the embodiment of the present disclosure can be suitably used, for example, as an optical film, a photothermal conversion material, an image forming material, a medical material, a lens having a function of absorbing or cutting near-infrared rays, an agricultural coating agent for the selective use of sunlight, protective glasses, or sunglasses, or as a material for forming these.

The composition according to the embodiment of the present disclosure is preferably colorless and transparent, but may be colored as long as the color tone cannot be clearly determined.

### (Compound Represented by Formula (1) or Tautomer Thereof)

The details of the compound represented by Formula (1) or the tautomer thereof (that is, the compound according to the embodiment of the present disclosure) contained in the composition according to the embodiment of the present disclosure are as described hereinbefore, and thus the description thereof will not be repeated.

The composition according to the embodiment of the present disclosure may contain only one type of the compound according to the embodiment of the present disclosure, or may contain two or more types of the compounds according to the embodiment of the present disclosure.

The content of the compound according to the embodiment of the present disclosure in the composition according to the embodiment of the present disclosure is not particularly limited, and can be appropriately set depending on the intended purpose.

The content of the compound according to the embodiment of the present disclosure in the composition according to the embodiment of the present disclosure is, for example, preferably 0.1% by mass to 90% by mass with respect to the total solid content mass of the composition. The lower limit of the compound according to the embodiment of the present disclosure is more preferably 0.5% by mass or more and still more preferably 1% by mass or more. The upper limit of the compound according to the embodiment of the present disclosure is more preferably 85% by mass or less and still more preferably 50% by mass or less.

### (Other Near Infrared Absorber)

The composition according to the embodiment of the present disclosure may further contain a compound having a near-infrared absorbing ability (hereinafter, also referred to as “other near infrared absorber”) other than the compound according to the embodiment of the present disclosure.

In a case where the composition according to the embodiment of the present disclosure contains the other near infrared absorber in addition to the compound according to the embodiment of the present disclosure, the composition according to the embodiment of the present disclosure makes it possible to ensure an ability to shield near-infrared rays in a wavelength range that cannot be sufficiently shielded by the compound according to the embodiment of the present disclosure alone.

Examples of the other near infrared absorber include a pyrrolopyrrole compound, a cyanine compound, a merocyanine compound, an oxonol compound, a squarylium compound, a croconium compound, a phthalocyanine compound, a naphthalocyanine compound, a quaterrylene compound, a pentarylene compound, a hexarylene compound, a diimmonium compound, a dithiol metal complex compound, a triarylmethane compound, a pyrromethene compound, an azomethine compound, an anthraquinone compound, and a benzodifuranone compound.

Examples of the pyrrolopyrrole compound include the compounds described in JP2009-263614A, JP2011-068731A, and WO2015/166873A.

Examples of the cyanine compound include the compounds described in JP2009-108267A, JP2002-194040A, JP2015-172004A, JP2015-172102A, JP2008-088426A, and WO2016/190162A.

Examples of the squarylium compound include the compounds described in JP2011-208101A, JP6065169B, WO2016/181987A, JP2015-176046A, WO2016/190162A, JP2016-074649A, JP2017-067963A, WO2017/135359A, JP2017-114956A, JP6197940B, and WO2016/120166A.

Examples of the croconium compound include the compounds described in JP2017-082029A.

Examples of the phthalocyanine compound include the compounds described in JP2012-077153A, JP2006-343631A, and JP2013-195480A.

Examples of the naphthalocyanine compound include the compounds described in JP2012-077153A.

In a case where the composition according to the embodiment of the present disclosure contains the other near infrared absorber, the composition according to the embodiment of the present disclosure may contain only one type of other near infrared absorber or may contain two or more types of other near infrared absorbers.

In a case where the composition according to the embodiment of the present disclosure contains the other near infrared absorber, the content of the other near infrared absorber is not particularly limited and is, for example, preferably 0.1% by mass to 90% by mass with respect to the total solid content mass of the composition.

The total content of the compound according to the embodiment of the present disclosure and the other near infrared absorber in the composition according to the embodiment of the present disclosure is not particularly limited and is, for example, preferably 0.1% by mass to 90% by mass with respect to the total solid content mass of the composition.

### (Ultraviolet Absorber)

The composition according to the embodiment of the present disclosure preferably contains an ultraviolet absorber.

The ultraviolet absorber is not particularly limited, and examples thereof include an aminobutadiene-based compound, a dibenzoylmethane-based compound, a salicylate-based compound, a benzophenone compound, a benzotriazole compound, an indole compound, and a triazine compound.

With regard to the details of these compounds, reference can be made to the descriptions in JP2012-208374A, JP2013-068814A, and JP2016-162946A, the contents of which are incorporated herein by reference.

For example, the compounds described in JP2003-128730A, JP2003-129033A, JP2014-077076A, JP2015-164994A, JP2015-168822A, JP2018-135282A, JP2018-168089A, JP2018-168278A, JP2018-188589A, JP2019-001767A, JP2020-023697A, JP2020-041013A, JP5518613B, JP5868465B, JP6301526B, JP6354665B, JP2017-503905A, WO2015/064674A, WO2015/064675A, WO2017/102675A, WO2017/122503A, WO2018/190281A, WO2018/216750A, WO2019/087983A, EP2379512B, and EP2951163B can be used as the ultraviolet absorber.

In addition, examples of commercially available products of the ultraviolet absorber include Tinuvin (registered trademark) P, Tinuvin (registered trademark) 234, Tinuvin (registered trademark) 326, Tinuvin (registered trademark) 571, and Tinuvin (registered trademark) 970 [all manufactured by BASF SE], which are benzotriazole-based compounds, and Tinuvin (registered trademark) 1577 and Tinuvin (registered trademark) 1600 [both manufactured by BASF SE], which are triazine compounds.

In addition, examples of commercially available products of the ultraviolet absorber having a polymerizable group include RUVA-93 [trade name, component: 2-[2-hydroxy-5-(2-methacryloyloxyethyl)phenyl]2H-benzo[d][1,2,3]triazole, manufactured by Otsuka Chemical Co., Ltd.].

In a case where the composition according to the embodiment of the present disclosure contains an ultraviolet absorber, the composition according to the embodiment of the present disclosure may contain only one type of ultraviolet absorber, or may contain two or more types of ultraviolet absorbers.

In a case where the composition according to the embodiment of the present disclosure contains an ultraviolet absorber, the content of the ultraviolet absorber is not particularly limited and is, for example, preferably 0.01% by mass to 10% by mass and more preferably 0.01% by mass to 5% by mass with respect to the total solid content mass of the composition.

### (Solvent)

The composition according to the embodiment of the present disclosure may contain a solvent.

The solvent is not particularly limited and can be appropriately selected, for example, in consideration of coating properties of the composition according to the embodiment of the present disclosure and solubility of each component contained in the composition according to the embodiment of the present disclosure.

Examples of the solvent include water and an organic solvent.

The solvent is preferably an organic solvent.

The compound according to the embodiment of the present disclosure contained in the composition according to the embodiment of the present disclosure tends to have excellent solubility in an organic solvent.

The organic solvent is not particularly limited, and examples thereof include an ester compound, an ether compound, a ketone compound, and an aromatic hydrocarbon compound.

With regard to the details of these compounds, reference can be made to the description in WO2015/166779A, the contents of which are incorporated herein by reference.

Specific examples of the organic solvent include dichloromethane, chloroform, methyl 3-methoxypropionate, methyl 3-ethoxypropionate, ethyl 3-ethoxypropionate, ethyl acetate, butyl acetate, cyclohexyl acetate, ethyl cellosolve acetate, ethyl carbitol acetate, butyl carbitol acetate, ethyl lactate, 1-methoxy-2-propyl acetate, diethylene glycol dimethyl ether, 2-heptanone, cyclohexanone, cyclopentanone, propylene glycol monomethyl ether, 3-methoxy-N,N-dimethylpropanamide, and 3-butoxy-N,N-dimethylpropanamide.

In a case where the composition according to the embodiment of the present disclosure contains a solvent, the composition according to the embodiment of the present disclosure may contain only one type of solvent or may contain two or more types of solvents.

In a case where the composition according to the embodiment of the present disclosure contains a solvent, the content of the solvent is not particularly limited and can be appropriately set depending on the intended purpose. In this regard, in a case where an aromatic hydrocarbon compound (for example, benzene, toluene, xylene, or ethylbenzene) is contained as the solvent, in consideration of an environmental aspect, the content of the aromatic hydrocarbon compound is, for example, preferably 50 parts per million (ppm) by mass or less, more preferably 10 ppm by mass or less, still more preferably 1 ppm by mass or less, and particularly preferably 0 ppm by mass with respect to the total mass of the solvent.

### (Surfactant)

The composition according to the embodiment of the present disclosure may contain a surfactant.

Examples of the surfactant include the surfactants described in paragraph of JP4502784B and paragraphs [0060] to [0071] of JP2009-237362A.

The surfactant is preferably at least one selected from the group consisting of a fluorine-based surfactant, a hydrocarbon-based surfactant, and a silicone-based surfactant. In addition, a nonionic surfactant other than the hydrocarbon-based surfactant is also preferable.

An acrylic compound, which has a molecular structure with a functional group containing a fluorine atom and in which a portion of the functional group containing a fluorine atom is cleaved upon application of heat and the fluorine atom volatilizes, can be preferably used as the fluorine-based surfactant. Examples of commercially available products of such a fluorine-based surfactant include MEGAFACE DS series manufactured by DIC Corporation [see, for example, The Chemical Daily (Feb. 22, 2016) and The Nikkei Business Daily (Feb. 23, 2016), such as MEGAFACE (registered trademark) DS-21 which will be described later.

In addition, a copolymer of a fluorine atom-containing vinyl ether compound having a fluorinated alkyl group or a fluorinated alkylene ether group, and a hydrophilic vinyl ether compound can also be preferably used as the fluorine-based surfactant.

In addition, a block polymer can also be used as the fluorine-based surfactant.

In addition, a fluorine-containing polymer compound containing a repeating unit derived from a (meth)acrylate compound having a fluorine atom and a repeating unit derived from a (meth)acrylate compound having 2 or more (preferably 5 or more) alkyleneoxy groups [preferably ethyleneoxy groups and/or propyleneoxy groups] can also be preferably used as the fluorine-based surfactant.

In addition, a fluorine-containing polymer having an ethylenically unsaturated bond-containing group in a side chain thereof can also be used as the fluorine-based surfactant. Examples of such a fluorine-based surfactant include MEGAFACE (registered trademark) RS-101, RS-102, RS-718-K, and RS-72-K (all manufactured by DIC Corporation), which are also listed as examples of commercially available products which will be described later.

From the viewpoint of environmental suitability, the fluorine-based surfactant is preferably a surfactant derived from a substitute material for a compound having a linear perfluoroalkyl group having 7 or more carbon atoms, such as perfluorooctanoic acid (PFOA) or perfluorooctanesulfonic acid (PFOS).

A commercially available product can be used as the fluorine-based surfactant.

Examples of commercially available products of the fluorine-based surfactant include MEGAFACE (registered trademark) F-171, F-172, F-173, F-176, F-177, F-141, F-142, F-143, F-144, F-437, F-475, F-477, F-479, F-482, F-551-A, F-552, F-554, F-555-A, F-556, F-557, F-558, F-559, F-560, F-561, F-565, F-563, F-568, F-575, F-780, EXP. MFS-330, EXP. MFS-578, EXP. MFS-578-2, EXP. MFS-579, EXP. MFS-586, EXP. MFS-587, EXP. MFS-628, EXP. MFS-631, EXP. MFS-603, R-41, R-41-LM, R-01, R-40, R-40-LM, RS-43, TF-1956, RS-90, R-94, RS-72-K, and DS-21 [all manufactured by DIC Corporation]; FLUORAD FC430, FC431, and FC171 [all manufactured by Sumitomo 3M Ltd.]; SURFLON (registered trademark) S-382, SC-101, SC-103, SC-104, SC-105, SC-1068, SC-381, SC-383, S-393, and KH-40 (all manufactured by AGC Seimi Chemical Co., Ltd.); POLYFOX PF636, PF656, PF6320, PF6520, and PF7002 [all manufactured by OMNOVA Solutions Inc.]; FTERGENT (registered trademark) 710FL, 710FM, 610FM, 601AD, 601ADH2, 602A, 215M, 245F, 251, 212M, 250, 209F, 222F, 208G, 710LA, 710FS, 730LM, 650AC, 681, and 683 [all manufactured by NEOS Company Limited]; and U-120E (manufactured by Unichem Co., Ltd.).

Examples of the hydrocarbon-based surfactant include glycerol, trimethylolpropane, trimethylolethane, and ethoxylates and propoxylates thereof (for example, glycerol propoxylate and glycerol ethoxylate).

In addition, examples of the hydrocarbon-based surfactant include polyoxyethylene lauryl ether, polyoxyethylene stearyl ether, polyoxyethylene oleyl ether, polyoxyethylene octylphenyl ether, polyoxyethylene nonylphenyl ether, polyethylene glycol dilaurate, polyethylene glycol distearate, and sorbitan fatty acid ester.

A commercially available product can be used as the hydrocarbon-based surfactant.

Examples of commercially available products of the hydrocarbon-based surfactant include PLURONIC (registered trademark) L10, L31, L61, L62, 10R5, 17R2, and 25R2 [all manufactured by BASF SE]; TETRONIC (registered trademark) 304, 701, 704, 901, 904, and 150R1 [all manufactured by BASF SE]; HYDROPALAT WE 3323 [manufactured by BASF SE]; SOLSPERSE (registered trademark) 20000 [manufactured by Nippon Lubrizol Corporation]; NCW-101, NCW-1001, and NCW-1002 [all manufactured by FUJIFILM Wako Pure Chemical Corporation]; PIONIN D-1105, D-6112, D-6112-W, and D-6315 [all manufactured by Takemoto Oil & Fat Co., Ltd.]; OLFINE (registered trademark) E1010 [manufactured by Nissin Chemical Co., Ltd.]; and SURFYNOL (registered trademark) 104, 400, and 440 [all manufactured by Nissin Chemical Co., Ltd.].

Examples of the silicone-based surfactant include a linear polymer consisting of a siloxane bond and a modified siloxane polymer with an organic group introduced into a side chain and/or a terminal thereof.

A commercially available product can be used as the silicone-based surfactant.

Examples of commercially available products of the silicone-based surfactant include MEGAFACE (registered trademark) EXP. S-309-2, EXP. S-315, EXP. S-503-2, and EXP. S-505-2 [all manufactured by DIC Corporation]; DC3PA, SH7PA, DC11PA, SH21PA, SH28PA, SH29PA, SH30PA, SH8400, and DOWSIL (registered trademark) 8032 ADDITIVE [all manufactured by DuPont Toray Specialty Materials K.K.]; X-22-4952, X-22-4272, X-22-6266, KF-351A, K354L, KF-355A, KF-945, KF-640, KF-642, KF-643, X-22-6191, X-22-4515, KF-6004, KP-341, KF-6001, KF-6002, KP-101, KP-103, KP-104, KP-105, KP-106, KP-109, KP-112, KP-120, KP-121, KP-124, KP-125, KP-301, KP-306, KP-310, KP-322, KP-323, KP-327, KP-341, KP-368, KP-369, KP-611, KP-620, KP-621, KP-626, and KP-652 [all manufactured by Shin-Etsu Chemical Co., Ltd.]; F-4440, TSF-4300, TSF-4445, TSF-4460, and TSF-4452 [all manufactured by Momentive Performance Materials, Inc.]; and BYK300, BYK306, BYK307, BYK310, BYK320, BYK323, BYK325, BYK330, BYK313, BYK315N, BYK331, BYK333, BYK345, BYK347, BYK348, BYK349, BYK370, BYK377, BYK378, and BYK323 (all manufactured by BYK-Chemie GmbH).

In a case where the composition according to the embodiment of the present disclosure contains a surfactant, the composition according to the embodiment of the present disclosure may contain only one type of surfactant or may contain two or more types of surfactants.

In a case where the composition according to the embodiment of the present disclosure contains a surfactant, the content of the surfactant is preferably 0.01% by mass to 3% by mass, more preferably 0.01% by mass to 1% by mass, and still more preferably 0.05% by mass to 0.8% by mass with respect to the total solid content mass of the composition.

### (Resin)

The composition according to the embodiment of the present disclosure can further contain a resin.

The resin is not particularly limited, and examples thereof include a (meth)acrylic resin, a polyester resin, a polycarbonate resin, a vinyl polymer (for example, a polydiene resin, a polyalkene resin, a polystyrene resin, a polyvinyl ether resin, a polyvinyl alcohol resin, a polyvinyl ketone resin, a polyfluorovinyl resin, or a polyvinyl bromide resin), a polythioether resin, a polyphenylene resin, a polyurethane resin, a polysulfonate resin, a nitroso polymer resin, a polysiloxane resin, a polysulfide resin, a polythioester resin, a polysulfone resin, a polysulfonamide resin, a polyamide resin, a polyimine resin, a polyurea resin, a polyphosphazene resin, a polysilane resin, a polysilazane resin, a polyfuran resin, a polybenzoxazole resin, a polyoxadiazole resin, a polybenzothiazinophenothiazine resin, a polybenzothiazole resin, a polypyrazinoquinoxaline resin, a polypyromellitimide resin, a polyquinoxaline resin, a polybenzimidazole resin, a polyoxoisoindoline resin, a polydioxoisoindoline resin, a polytriazine resin, a polypyridazine resin, a polypiperazine resin, a polypyridine resin, a polypiperidine resin, a polytriazole resin, a polypyrazole resin, a polypyrrolidine resin, a polycarborane resin, a polyoxabicyclononane resin, a polydibenzofuran resin, a polyphthalide resin, a polyacetal resin, a polyimide resin, an olefin resin, a cyclic olefin resin, an epoxy resin, and a cellulose acylate resin.

With regard to the details of the resin, reference can be made to, for example, the description in paragraphs [0075] to [0097] of JP2009-263616A, the contents of which are incorporated herein by reference.

From the viewpoint of good compatibility with the compound according to the embodiment of the present disclosure and easily obtaining a film with suppressed surface unevenness in a case where the film is formed, the resin is preferably at least one resin selected from the group consisting of a (meth)acrylic resin, a polyester resin, a polystyrene resin, a polyurethane resin, a polycarbonate resin, an epoxy resin, and a cellulose acylate resin, and more preferably at least one resin selected from the group consisting of a (meth)acrylic resin, a polyester resin, a polystyrene resin, a polyurethane resin, an epoxy resin, and a cellulose acylate resin.

Examples of commercially available products of the (meth)acrylic resin include SK DYNE series [product example: SK DYNE (registered trademark) SF2147, manufactured by Soken Chemical & Engineering Co., Ltd.].

Examples of commercially available products of the polyester resin include VYLON series [product example: VYLON 500, manufactured by Toyobo Co., Ltd.].

The polystyrene resin is preferably a resin containing 50% by mass or more of a repeating unit derived from a styrene-based monomer with respect to all repeating units, more preferably a resin containing 70% by mass or more of a repeating unit derived from a styrene-based monomer with respect to all repeating units, and still more preferably a resin containing 85% by mass or more of a repeating unit derived from a styrene-based monomer with respect to all repeating units.

Specific examples of the styrene-based monomer include styrene and a styrene derivative. Here, the “styrene derivative” refers to a compound in which another group is bonded to styrene.

Specific examples of the styrene derivative include alkylstyrenes (for example, o-methylstyrene, m-methylstyrene, p-methylstyrene, 2,4-dimethylstyrene, o-ethylstyrene, and p-ethylstyrene). In addition, specific examples of the styrene derivative include substituted styrenes in which a hydroxyl group, an alkoxy group, a carboxyl group, a halogen, or the like is introduced into a benzene nucleus of styrene (for example, hydroxystyrene, tert-butoxystyrene, vinylbenzoic acid, o-chlorostyrene, and p-chlorostyrene).

The polystyrene resin may contain a repeating unit derived from a monomer other than the styrene-based monomer. Examples of the other monomer include an alkyl (meth)acrylate, an unsaturated carboxylic acid monomer, an unsaturated dicarboxylic acid anhydride monomer, an unsaturated nitrile monomer, and a conjugated diene.

Specific examples of the alkyl (meth)acrylate include methyl (meth)acrylate, cyclohexyl (meth)acrylate, methylphenyl (meth)acrylate, and isopropyl (meth)acrylate.

Specific examples of the unsaturated carboxylic acid monomer include methacrylic acid, acrylic acid, itaconic acid, maleic acid, fumaric acid, and cinnamic acid.

Specific examples of the unsaturated dicarboxylic acid anhydride monomer include maleic acid anhydride and anhydrides of itaconic acid, ethyl maleic acid, methyl itaconic acid, and chloromaleic acid.

Specific examples of the unsaturated nitrile monomer include acrylonitrile and methacrylonitrile.

Specific examples of the conjugated diene include 1,3-butadiene, 2-methyl-1,3-butadiene (isoprene), 2,3-dimethyl-1,3-butadiene, 1,3-pentadiene, and 1,3-hexadiene.

Examples of commercially available products of the polystyrene resin include AS-70 [acrylonitrile/styrene copolymer resin, manufactured by NIPPON STEEL Chemical & Material Co., Ltd.] and SMA2000P [styrene/maleic acid copolymer, manufactured by Kawahara Petrochemical Co., Ltd.].

Examples of the epoxy resin include a bisphenol A type epoxy resin, a bisphenol F type epoxy resin, a phenol novolac type epoxy resin, a cresol novolac type epoxy resin, and an aliphatic epoxy resin.

Examples of commercially available products of the bisphenol A type epoxy resin include JER827, JER828, JER834, JER1001, JER1002, JER1003, JER1055, JER1007, JER1009, and JER1010 [all manufactured by Mitsubishi Chemical Corporation]; and EPICLON (registered trademark) 860, EPICLON (registered trademark) 1050, EPICLON (registered trademark) 1051, and EPICLON (registered trademark) 1055 [all manufactured by DIC Corporation].

Examples of commercially available products of the bisphenol F type epoxy resin include JER806, JER807, JER4004, JER4005, JER4007, and JER4010 [all manufactured by Mitsubishi Chemical Corporation]; EPICLON (registered trademark) 830 and EPICLON (registered trademark) 835 [both manufactured by DIC Corporation]; and LCE-21 and RE-602S [both manufactured by Nippon Kayaku Co., Ltd.].

Examples of commercially available products of the phenol novolac type epoxy resin include JER152, JER154, JER157S70, and JER157S65 [all manufactured by Mitsubishi Chemical Corporation]; and EPICLON (registered trademark) N-740, EPICLON (registered trademark) N-770, and EPICLON (registered trademark) N-775 [all manufactured by DIC Corporation].

Examples of commercially available products of the cresol novolac type epoxy resin include EPICLON (registered trademark) N-660, EPICLON (registered trademark) N-665, EPICLON (registered trademark) N-670, EPICLON (registered trademark) N-673, EPICLON (registered trademark) N-680, EPICLON (registered trademark) N-690, and EPICLON (registered trademark) N-695 [all manufactured by DIC Corporation]; and EOCN-1020 [manufactured by Nippon Kayaku Co., Ltd.].

The cellulose acylate resin is, for example, preferably the cellulose acylate described in paragraphs [0016] to [0021] of JP2012-215689A.

The resin is preferably an alkali-soluble resin.

In the present disclosure, the term “alkali-soluble” refers to being soluble in a 1 mol/L sodium hydroxide solution at 25° C. In addition, the term “soluble” refers to that 0.1 g or more is dissolved in 100 mL of a solvent.

The alkali-soluble resin is preferably a resin having a group that promotes alkali solubility (hereinafter, also referred to as an “acid group”).

Examples of the acid group include a carboxy group, a phosphate group, a sulfonate group, and a phenolic hydroxy group.

Above all, the acid group is preferably a carboxy group.

In a case where the resin has an acid group, the resin may have only one type of acid group or may have two or more types of acid groups.

The alkali-soluble resin is preferably a polymer having a carboxy group in a side chain thereof.

Examples of the alkali-soluble resin include a methacrylic acid copolymer, an itaconic acid copolymer, a crotonic acid copolymer, a maleic acid copolymer, a partially esterified maleic acid copolymer, an alkali-soluble phenol resin such as a novolac type resin, an acidic cellulose derivative having a carboxy group in a side chain thereof, and a product obtained by adding an acid anhydride to a polymer having a hydroxy group.

The alkali-soluble resin is particularly preferably a copolymer of (meth)acrylic acid and another monomer copolymerizable with the (meth)acrylic acid [that is, a (meth)acrylic acid copolymer].

Examples of the other monomer copolymerizable with (meth)acrylic acid include an alkyl (meth)acrylate, an aryl (meth)acrylate, and a vinyl compound.

Specific examples of the other monomer copolymerizable with (meth)acrylic acid include methyl (meth)acrylate, ethyl (meth)acrylate, propyl (meth)acrylate, butyl (meth)acrylate, isobutyl (meth)acrylate, pentyl (meth)acrylate, hexyl (meth)acrylate, octyl (meth)acrylate, phenyl (meth)acrylate, benzyl (meth)acrylate, tolyl (meth)acrylate, naphthyl (meth)acrylate, cyclohexyl (meth)acrylate, styrene, α-methylstyrene, vinyl toluene, glycidyl methacrylate, acrylonitrile, vinyl acetate, N-vinylpyrrolidone, tetrahydrofurfuryl methacrylate, a polystyrene macromonomer, and a polymethyl methacrylate macromonomer.

In addition, examples of the other monomer copolymerizable with (meth)acrylic acid include N-substituted maleimides (for example, N-phenylmaleimide and N-cyclohexylmaleimide) described in JP1998-300922A (JP-H10-300922A).

In the (meth)acrylic acid copolymer, the number of types of other monomers copolymerizable with (meth)acrylic acid may be only one type or may be two or more types.

In addition, preferred examples of the alkali-soluble resin also include a benzyl (meth)acrylate/(meth)acrylic acid copolymer, a benzyl (meth)acrylate/(meth)acrylic acid/2-hydroxyethyl (meth)acrylate copolymer, and a multi-component copolymer consisting of benzyl (meth)acrylate/(meth)acrylic acid/another monomer.

In addition, preferred examples of the alkali-soluble resin also include a copolymer obtained by copolymerization of 2-hydroxyethyl (meth)acrylate, as well as a 2-hydroxypropyl (meth)acrylate/polystyrene macromonomer/benzyl methacrylate/methacrylic acid copolymer, a 2-hydroxy-3-phenoxypropyl acrylate/polymethyl methacrylate macromonomer/benzyl methacrylate/methacrylic acid copolymer, a 2-hydroxyethyl methacrylate/polystyrene macromonomer/methyl methacrylate/methacrylic acid copolymer, and a 2-hydroxyethyl methacrylate/polystyrene macromonomer/benzyl methacrylate/methacrylic acid copolymer described in JP1995-140654A (JP-H07-140654A).

With regard to the details of the alkali-soluble resin, reference can be made to, for example, the descriptions in JP2012-208494A and JP2012-198408A, the contents of which are incorporated herein by reference.

The acid value of the alkali-soluble resin is not particularly limited and is, for example, preferably 30 mgKOH/g to 30 mgKOH/g. The lower limit of the acid value of the alkali-soluble resin is more preferably 50 mgKOH/g or more and still more preferably 70 mgKOH/g or more. The upper limit of the acid value of the alkali-soluble resin is more preferably 150 mgKOH/g or less and still more preferably 120 mgKOH/g or less.

In the present disclosure, the acid value is a value measured according to the method described in JIS K0070: 1992.

The resin may have a polymerizable group.

In a case where the composition according to the embodiment of the present disclosure contains a resin having a polymerizable group, the composition according to the embodiment of the present disclosure can form a film having hardness, for example, even in a case where the composition does not contain a polymerizable compound which will be described later.

Examples of the polymerizable group include a (meth)allyl group and a (meth)acryloyl group.

Examples of commercially available products of the resin having a polymerizable group include DIANAL BR Series [type of resin: polymethyl methacrylate (PMMA), product examples: DIANAL (registered trademark) BR-80, BR-83, and BR-87, manufactured by Mitsubishi Chemical Corporation], Photomer 6173 [type of resin: COOH-containing polyurethane acrylic oligomer, manufactured by Diamond Shamrock Co., Ltd.], VISCOAT R-264 and KS RESIST 106 [both manufactured by Osaka Organic Chemical Industry Ltd.], CYCLOMER P Series [product example: CYCLOMER (registered trademark) P (ACA) Z230AA, manufactured by Daicel Corporation] and PLACCEL CF200 Series [manufactured by Daicel Corporation], EBECRYL (registered trademark) 3800 [manufactured by Daicel-Allnex Ltd.], and ACRYCURE (registered trademark) RD-F8 [manufactured by Nippon Shokubai Co., Ltd.].

The weight-average molecular weight (Mw) of the resin other than the epoxy resin is not particularly limited and is, for example, preferably 3,000 or more, more preferably 4,000 or more, and still more preferably 5,000 or more. In addition, the weight-average molecular weight (Mw) of the resin other than the epoxy resin is, for example, preferably 2,000,000 or less, more preferably 1,000,000 or less, and still more preferably 500,000 or less.

The weight-average molecular weight (Mw) of the epoxy resin is preferably 100 or more and more preferably 200 or more. In addition, the weight-average molecular weight (Mw) of the epoxy resin is, for example, preferably 2,000,000 or less, more preferably 1,000,000 or less, and still more preferably 500,000 or less.

The total light transmittance of the resin is preferably 80% or more, more preferably 85% or more, and still more preferably 90% or more. In the present disclosure, the total light transmittance of the resin is a value measured based on the contents described in “The Fourth Series of Experimental Chemistry 29 Polymer Material” (Maruzen Publishing Co., Ltd., 1992), pp. 225 to 232, edited by The Chemical Society of Japan.

In a case where the composition according to the embodiment of the present disclosure contains a resin, the composition according to the embodiment of the present disclosure may contain only one type of resin or may contain two or more types of resins.

In a case where the composition according to the embodiment of the present disclosure contains a resin, the content of the resin is not particularly limited and is, for example, preferably 1% by mass to 99.9% by mass with respect to the total solid content mass of the composition. The lower limit of the content of the resin is more preferably 5% by mass or more and still more preferably 7% by mass or more. The upper limit of the content of the resin is more preferably 99% by mass or less and still more preferably 95% by mass or less.

### (Polymerizable Compound)

The composition according to the embodiment of the present disclosure can contain a polymerizable compound.

In a case where the composition according to the embodiment of the present disclosure contains a polymerizable compound, the composition according to the embodiment of the present disclosure can be used as a curable composition that is cured by the application of energy (so-called curable composition).

The polymerizable compound may be any compound that can be polymerized and cured by the application of energy, and is not particularly limited. The polymerizable compound is preferably a compound having a polymerizable group.

The polymerizable group is preferably a group having an ethylenically unsaturated bond.

Specific examples of the polymerizable group include a vinyl group, a (meth)allyl group, a (meth)acryloyl group, a (meth)acryloyloxy group, a (meth)acryloylamino group, and a vinylphenyl group.

The polymerizable compound may be any of a monomer, a prepolymer (that is, a dimer, a trimer, or an oligomer), a mixture thereof, a (co)polymer of a compound selected from the monomer and the prepolymer, or the like.

Examples of the monomer include an unsaturated carboxylic acid, an ester of the unsaturated carboxylic acid, and an amide of the unsaturated carboxylic acid.

Specific examples of the unsaturated carboxylic acid include acrylic acid, methacrylic acid, itaconic acid, crotonic acid, isocrotonic acid, and maleic acid.

The polymerizable compound is preferably at least one selected from the group consisting of a (meth)acrylate-based monomer and a styrene-based monomer.

Specific examples of the (meth)acrylate-based monomer include methyl (meth)acrylate, ethyl (meth)acrylate, butyl (meth)acrylate, 2-hydroxyethyl (meth)acrylate, glycidyl (meth)acrylate, benzyl (meth)acrylate, 2-(2-phenoxy)ethyl (meth)acrylate, 2-ethylhexyl (meth)acrylate, n-octyl (meth)acrylate, isooctyl (meth)acrylate, n-nonyl (meth)acrylate, isononyl (meth)acrylate, n-decyl (meth)acrylate, isodecyl (meth)acrylate, n-dodecyl (meth)acrylate, n-tridecyl (meth)acrylate, n-tetradecyl (meth)acrylate, n-hexadecyl (meth)acrylate, stearyl (meth)acrylate, isobornyl (meth)acrylate, dicyclopentenyl (meth)acrylate, dicyclopentanyl (meth)acrylate, dicyclopentenyloxyethyl (meth)acrylate, 1-hydroxyheptyl (meth)acrylate, 1-hydroxybutyl (meth)acrylate, 1-hydroxypentyl, 2-hydroxybutyl (meth)acrylate, ethylene glycol diacrylate, ethylene glycol dimethacrylate, polyethylene glycol diacrylate, polyethylene glycol dimethacrylate, ethoxylated bisphenol A diacrylate, ethoxylated bisphenol A dimethacrylate, ethoxylated trimethylolpropane triacrylate, ethoxylated trimethylolpropane trimethacrylate, ethoxylated glycerin triacrylate, ethoxylated glycerin trimethacrylate, ethoxylated pentaerythritol tetraacrylate, ethoxylated pentaerythritol tetramethacrylate, ethoxylated dipentaerythritol hexaacrylate, polyglycerin monoethylene oxide polyacrylate, polyglycerin polyethylene glycol polyacrylate, dipentaerythritol hexaacrylate, dipentaerythritol hexamethacrylate, neopentyl glycol diacrylate, neopentyl glycol dimethacrylate, pentaerythritol triacrylate, pentaerythritol trimethacrylate, trimethylolpropane triacrylate, trimethylolpropane trimethacrylate, tricyclodecane dimethanol diacrylate, tricyclodecane dimethanol dimethacrylate, 1,6-hexanediol diacrylate, and 1,6-hexanediol dimethacrylate.

As the (meth)acrylate-based monomer, ethylene oxide-modified pentaerythritol tetraacrylate [example of commercially available product: NK ESTER ATM-35E, manufactured by Shin-Nakamura Chemical Co., Ltd.], dipentaerythritol triacrylate [example of commercially available product: KAYARAD (registered trademark) D-330, manufactured by Nippon Kayaku Co., Ltd.], dipentaerythritol tetraacrylate [example of commercially available product: KAYARAD (registered trademark) D-320, manufactured by Nippon Kayaku Co., Ltd.], dipentaerythritol penta(meth)acrylate [example of commercially available product: KAYARAD (registered trademark) D-310, manufactured by Nippon Kayaku Co., Ltd.], diglycerin ethylene oxide-modified (meth)acrylate [example of commercially available product: M-460, manufactured by Toagosei Co., Ltd.], pentaerythritol tetraacrylate [example of commercially available product: A-TMMT, manufactured by Shin-Nakamura Chemical Co., Ltd.], and 1,6-hexanediol diacrylate [example of commercially available product: KAYARAD (registered trademark) HDDA, manufactured by Nippon Kayaku Co., Ltd.] can be preferably used, and oligomer types of those (meth)acrylate-based monomer compounds can also be used.

In addition, as the (meth)acrylate-based monomer, RP-1040 [manufactured by Nippon Kayaku Co., Ltd.], dipentaerythritol hexa(meth)acrylate [examples of commercially available products: KAYARAD (registered trademark) DPHA manufactured by Nippon Kayaku Co., Ltd. and A-DPH-12E manufactured by Shin-Nakamura Chemical Co., Ltd.], and a compound containing a structure in which (meth)acryloyl groups of those monomers are bonded through an ethylene glycol residue, a propylene glycol residue, or the like can be preferably used, and oligomer types of those (meth)acrylate-based monomer compounds can also be used.

Specific examples of the styrene-based monomer include styrene, methylstyrene, dimethylstyrene, trimethylstyrene, ethylstyrene, fluorostyrene, chlorostyrene, methoxystyrene, tert-butoxystyrene, and divinylbenzene.

The polymerizable compound is particularly preferably at least one selected from the group consisting of methyl (meth)acrylate, ethyl (meth)acrylate, butyl (meth)acrylate, 2-hydroxyethyl (meth)acrylate, benzyl (meth)acrylate, 2-ethylhexyl (meth)acrylate, dipentaerythritol hexaacrylate, and pentaerythritol triacrylate, for example, from the viewpoint of being in a liquid state at normal temperature.

The polymerizable compound containing a group having an ethylenically unsaturated bond may further have an acid group such as a carboxy group, a sulfonate group, a phosphate group, or a phosphonate group.

The polymerizable compound containing an acid group and a group having an ethylenically unsaturated bond is, for example, preferably a polyfunctional monomer in which an unreacted hydroxy group of an aliphatic polyhydroxy compound is reacted with a non-aromatic carboxylic acid anhydride to have an acid group, and particularly preferably a polyfunctional monomer in which an unreacted hydroxy group of an aliphatic polyhydroxy compound is reacted with a non-aromatic carboxylic acid anhydride to have an acid group and the aliphatic polyhydroxy compound is at least one of pentaerythritol or dipentaerythritol. The polymerizable compound containing an acid group and a group having an ethylenically unsaturated bond is described, for example, in paragraphs [0025] to [0030] of JP2004-239942A. These descriptions are incorporated herein by reference.

Examples of commercially available products of the polymerizable compound containing an acid group and a group having an ethylenically unsaturated bond include M-305, M-510, and M-520 of ARONIX series which are polybasic acid-modified acrylic oligomers manufactured by Toagosei Co., Ltd.

The acid value of the polymerizable compound containing an acid group and a group having an ethylenically unsaturated bond is not particularly limited and is, for example, preferably 0.1 mgKOH/g to 40 mgKOH/g. The lower limit of the acid value of the polymerizable compound is more preferably 5 mgKOH/g or more. The upper limit of the acid value of the polymerizable compound is more preferably 30 mgKOH/g or less.

A compound having a caprolactone structure is also preferable as the polymerizable compound.

With regard to the compound having a caprolactone structure, reference can be made to, for example, the description in JP2013-253224A, the contents of which are incorporated herein by reference.

Examples of commercially available products of the compound having a caprolactone structure include SR-494 (manufactured by Sartomer Company Inc.), which is a tetrafunctional acrylate having 4 ethyleneoxy chains, DPCA-60 (manufactured by Nippon Kayaku Co., Ltd.), which is a hexafunctional acrylate having 6 pentyleneoxy chains, and TPA-330 (manufactured by Nippon Kayaku Co., Ltd.), which is a trifunctional acrylate having 3 isobutyleneoxy chains.

With regard to the polymerizable compound, the details of how to use the polymerizable compound, such as what type of structure the polymerizable compound has, whether to use a single polymerizable compound, whether to use two or more types of polymerizable compounds in combination, and what amount to use, can be optionally set according to the final performance design of the composition (preferably the curable composition).

For example, from the viewpoint of sensitivity, the polymerizable compound is preferably a compound having a structure with a large amount of polymerizable groups per molecule, and in many cases, the polymerizable compound is preferably a bi- or higher functional compound.

For example, from the viewpoint of film hardness, a tri- or higher functional compound [for example, a hexafunctional (meth)acrylate monomer] may be used as the polymerizable compound.

For example, it is also effective to select a polymerizable compound in consideration of compatibility, dispersibility, and the like with each component contained in the composition.

Compounds having different functional groups and/or different polymerizable groups [for example, a (meth)acrylate compound, a styrene compound, and a vinyl ether compound] may be used in combination as the polymerizable compound.

The molecular weight of the polymerizable compound is not particularly limited and is, for example, preferably 100 to 3000, more preferably 250 to 2600, still more preferably 280 to 2200, and particularly preferably 300 to 2200.

In a case where the composition according to the embodiment of the present disclosure contains a polymerizable compound, the composition according to the embodiment of the present disclosure may contain only one type of polymerizable compound or may contain two or more types of polymerizable compounds.

In a case where the composition according to the embodiment of the present disclosure contains a polymerizable compound, the content of the polymerizable compound is not particularly limited and is, for example, preferably 1% by mass to 90% by mass with respect to the total solid content mass of the composition. The lower limit of the content of the polymerizable compound is more preferably 15% by mass or more and still more preferably 40% by mass or more. The upper limit of the content of the polymerizable compound is more preferably 80% by mass or less and still more preferably 75% by mass or less.

### (Polymerization Initiator)

The composition according to the embodiment of the present disclosure (preferably a curable composition) can contain a polymerization initiator.

The polymerization initiator may be any compound capable of generating an initiating species required for the polymerization reaction by the application of energy, and is not particularly limited. A known polymerization initiator can be used as the polymerization initiator.

Examples of the polymerization initiator include a photopolymerization initiator and a thermal polymerization initiator.

The polymerization initiator is preferably a photopolymerization initiator.

The photopolymerization initiator is, for example, preferably a photopolymerization initiator having photosensitivity to light rays from an ultraviolet range to a visible range. In addition, the photopolymerization initiator may be an activator that generates active radicals through some interaction with the photoexcited sensitizer.

Examples of the photopolymerization initiator include a halogenated hydrocarbon derivative (for example, a compound having a triazine skeleton or a compound having an oxadiazole skeleton), an acylphosphine compound, a hexaarylbiimidazole, an oxime compound (for example, an oxime ester compound), an organic peroxide, a thio compound, a ketone compound, an aromatic onium salt, an aminoacetophenone compound, and a hydroxyacetophenone compound.

Examples of the acylphosphine compound include the acylphosphine-based initiators described in JP4225898B.

Examples of the oxime compound include the compounds described in JP2001-233842A, the compounds described in JP2000-080068A, the compounds described in JP2006-342166A, and the compounds described in paragraphs [0073] to [0075] of JP2016-006475A. Among the oxime compounds, an oxime ester compound is preferred.

Examples of the aminoacetophenone compound include the compounds described in JP2009-191179A and the aminoacetophenone-based initiators described in JP1998-291969A (JP-H10-291969A).

Examples of the thermal polymerization initiator include an azo-based compound, an organic peroxide, and an inorganic peroxide.

Specific examples of the azo-based compound include dimethyl 2,2′-azobis(isobutyrate), 2,2′-azobisisobutyronitrile, 2,2′-azobis(2,4-dimethyl-4-methoxyvaleronitrile), 2,2′-azobis(2,4-dimethylvaleronitrile), dimethyl 2,2′-azobis(2-methylpropionate), 2,2′-azobis(2-methylbutyronitrile), 1,1′-azobis(cyclohexane-1-carbonitrile), 2,2′-azobis(N-butyl-2-methylpropionamide), dimethyl 1,1′-azobis(1-cyclohexanecarboxylate), and 2,2′-azobis[2-(2-imidazolin-2-yl)propane] dihydrochloride.

Specific examples of the organic peroxide include 1,1-di(tert-hexylperoxy)cyclohexane, 1,1-di(tert-butylperoxy)cyclohexane, 2,2-di(4,4-di-(tert-butylperoxy)cyclohexyl)propane, tert-hexyl peroxy isopropyl monocarbonate, tert-butyl peroxy-3,5,5-trimethyl hexanoate, tert-butyl peroxy laurate, dicumyl peroxide, di-tert-butyl peroxide, tert-butyl peroxy-2-ethyl hexanoate, tert-hexyl peroxy-2-ethyl hexanoate, cumene hydroperoxide, and tert-butyl hydroperoxide.

Specific examples of the inorganic peroxide include potassium persulfate, ammonium persulfate, and hydrogen peroxide.

The polymerization initiator may be a synthetic product or a commercially available product.

Examples of commercially available products of the photopolymerization initiator include IRGACURE (registered trademark) OXE01 [manufactured by BASF SE], TR-PBG-304 [manufactured by Changzhou Tronly New Electronic Materials Co., Ltd.], and ADEKA ARKLS (registered trademark) NCI-831 and ADEKA ARKLS (registered trademark) NCI-930 [both manufactured by ADEKA Corporation].

In addition, examples of commercially available products of the photopolymerization initiator which is a hydroxyacetophenone compound include Omnirad (registered trademark) 184, Omnirad (registered trademark) 1173, Omnirad (registered trademark) 2959, and Omnirad (registered trademark) 127 [all manufactured by IGM Resins B.V.]. Examples of commercially available products of the photopolymerization initiator which is an aminoacetophenone compound include Omnirad (registered trademark) 907, Omnirad (registered trademark) 369, Omnirad (registered trademark) 369E, and Omnirad (registered trademark) 379EG [all manufactured by IGM Resins B.V.]. Examples of commercially available products of the photopolymerization initiator which is an acylphosphine compound include Omnirad (registered trademark) 819 and Omnirad (registered trademark) TPO [both manufactured by IGM Resins B.V.]. Examples of commercially available products of the photopolymerization initiator which is an oxime compound include Irgacure (registered trademark) OXE01, Irgacure (registered trademark) OXE02 (manufactured by BASF SE), and Irgacure (registered trademark) OXE03 [all manufactured by BASF SE].

In a case where the composition according to the embodiment of the present disclosure contains a polymerization initiator, the composition according to the embodiment of the present disclosure may contain only one type of polymerization initiator or may contain two or more types of polymerization initiators.

In a case where the composition according to the embodiment of the present disclosure contains a polymerization initiator, the content of the polymerization initiator is not particularly limited and is, for example, preferably 0.1% by mass to 20% by mass with respect to the total solid content mass of the composition. The lower limit of the content of the polymerization initiator is more preferably 0.3% by mass or more and still more preferably 0.4% by mass or more. The upper limit of the content of the polymerization initiator is more preferably 15% by mass or less and still more preferably 10% by mass or less.

### (Other Components)

The composition according to the embodiment of the present disclosure may contain components other than those described above (so-called other components), as necessary, as long as the effect of the present disclosure is not impaired.

Examples of other components include various additives.

Examples of the additive include additives such as an antifading agent, a preservative, a fungicide, and an antistatic agent.

### [Laminate]

The laminate according to the embodiment of the present disclosure includes a support and a film provided on the support and formed of the composition according to the embodiment of the present disclosure described above.

The laminate according to the embodiment of the present disclosure includes a film formed of the composition according to the embodiment of the present disclosure containing the compound according to the embodiment of the present disclosure, and therefore has maximum absorption in a near-infrared range on a wavelength side longer than 850 nm and exhibits excellent light resistance.

The laminate according to the embodiment of the present disclosure can be suitably used as a near-infrared shielding material.

The support preferably has transparency within a range that does not impair optical performance.

In the present disclosure, the expression “support has transparency” means that the support is optically transparent, and specifically refers to that the total light transmittance of the support is 85% or more.

The total light transmittance of the support is preferably 90% or more and more preferably 95% or more.

The support is not particularly limited, and examples thereof include glass and a resin film.

Examples of the resin which is a material of the resin film include an ester resin [for example, polyethylene terephthalate (PET), polyethylene naphthalate (PEN), polybutylene terephthalate (PBT), or polycyclohexane dimethylene terephthalate (PCT)], an olefin resin [for example, polypropylene (PP) or polyethylene (PE)], polyvinyl chloride (PVA), and tricellulose acetate (TAC).

Above all, for example, from the viewpoint of general-purpose properties, PET is preferable as the resin which is a material of the resin film.

The thickness of the support is not particularly limited, and can be appropriately selected depending on the intended use or purpose.

In general, the thickness of the support is preferably 5 μm to 2500 μm and more preferably 20 μm to 500 μm.

The support may have peelability.

The laminate according to the embodiment of the present disclosure, which includes a support having peelability, is preferably used for, for example, a polarizing plate.

Examples of the support having peelability include a support in which one side or both sides have been subjected to a surface treatment using a peeling treatment agent (so-called easy peeling treatment).

Examples of the peeling treatment agent include a silicone-based peeling treatment agent (for example, silicone), a wax-based peeling treatment agent (for example, paraffin wax), and a fluorine-based peeling treatment agent (for example, a fluorine-based resin).

The film formed of the composition according to the embodiment of the present disclosure may be a dried product of the composition according to the embodiment of the present disclosure or may be a cured product of the composition according to the embodiment of the present disclosure.

The thickness of the film formed of the composition according to the embodiment of the present disclosure is not particularly limited and is, for example, preferably 0.1 μm to 2500 μm and more preferably 1 μm to 500 μm.

### —Method for Producing Laminate—

The method for producing the laminate according to the embodiment of the present disclosure is not particularly limited, and a known production method in the related art can be adopted.

The laminate according to the embodiment of the present disclosure can be produced, for example, by applying the composition according to the embodiment of the present disclosure onto a support having transparency to form a composition layer, and then applying energy to cure the composition layer.

The method of applying energy to the composition layer is not particularly limited, and examples thereof include heating and irradiation with light. The method of applying energy is preferably irradiation with light and more preferably irradiation with ultraviolet rays.

In a case where the composition layer is cured by irradiation with ultraviolet rays, for example, an ultraviolet lamp can be used for irradiation with ultraviolet rays.

A light irradiation amount of ultraviolet rays is not particularly limited and is, for example, preferably 10 mJ/cm² to 1000 mJ/cm². In a case where the light irradiation amount of ultraviolet rays is within the above-described range, the composition layer tends to be cured more suitably.

In a case of irradiation with ultraviolet rays, for the purpose of suppressing curing inhibition caused by oxygen and further accelerating surface curing of the composition layer, an inert gas such as nitrogen gas may be sent into a spatial domain where the irradiation with ultraviolet rays is carried out to replace the air in the ultraviolet irradiated region with the inert gas to reduce an oxygen concentration.

The oxygen concentration in the spatial domain where the irradiation with ultraviolet rays is carried out is not particularly limited and is, for example, preferably 0.01% to 5%.

The temperature at which the composition layer is cured can be increased for the purpose of accelerating the curing reaction of the composition layer. The temperature at which the composition layer is cured is, for example, preferably 25° C. to 100° C., more preferably 30° C. to 80° C., and still more preferably 40° C. to 70° C., from the viewpoint of further accelerating the curing reaction of the composition layer.

In the method for producing the laminate according to the embodiment of the present disclosure, in a case where the composition according to the embodiment of the present disclosure contains a solvent, it is preferable from the viewpoint of improving curing properties of the composition layer to dry the composition layer to reduce the amount of solvent before curing the composition layer.

The method of drying the composition layer is not particularly limited, and a known drying method in the related art can be adopted.

Examples of the method of drying the composition layer include a method of blowing hot air onto the composition layer, a method of passing the composition layer through a drying zone controlled at a predetermined temperature, and a method of transporting the composition layer by a transport roll equipped with a heater.

### [Optical Filter]

The optical filter according to the embodiment of the present disclosure contains the compound according to the embodiment of the present disclosure described above.

The optical filter according to the embodiment of the present disclosure can function as a near-infrared shielding filter.

In the present disclosure, the term “near-infrared shielding filter” refers to a filter that transmits light having a wavelength in a visible range (so-called visible light) and shields at least a part of light having a wavelength in a near infrared range (so-called near-infrared ray). Here, the near-infrared shielding filter may be a filter that transmits light having a wavelength in the entire wavelength range of a visible range, or may be a filter that transmits light having a wavelength in a specific wavelength range of a visible range and shields light having a wavelength in another specific wavelength range of the visible range, among light having a wavelength in the visible range.

The thickness of the optical filter according to the embodiment of the present disclosure is not particularly limited, and can be appropriately selected depending on the intended purpose.

The thickness of the optical filter according to the embodiment of the present disclosure is, for example, preferably 0.1 μm to 2500 μm and more preferably 1 μm to 500 μm.

At a thickness of 200 μm or less, the optical filter according to the embodiment of the present disclosure has a light transmittance of preferably 70% or more, more preferably 80% or more, and still more preferably 90% or more, for all wavelengths within a wavelength range of 400 nm to 650 nm.

The optical filter according to the embodiment of the present disclosure preferably has a maximal absorption wavelength in a wavelength range of 700 nm to 1200 nm.

### —Method for Producing Optical Filter—

The optical filter according to the embodiment of the present disclosure can be produced using the composition according to the embodiment of the present disclosure containing at least the compound according to the embodiment of the present disclosure and a resin (hereinafter, also referred to as “resin composition”), or the composition according to the embodiment of the present disclosure containing at least the compound according to the embodiment of the present disclosure and a polymerizable compound (so-called curable composition).

Hereinafter, an example of a suitable method for producing the optical filter according to the embodiment of the present disclosure will be described. In this regard, the description of items common to the compound according to the embodiment of the present disclosure described above and the composition according to the embodiment of the present disclosure described above will not be repeated.

The optical filter according to the embodiment of the present disclosure can be produced, for example, by a method including a step of applying the composition according to the embodiment of the present disclosure (preferably the resin composition or the curable composition) onto a support to form a composition layer.

The support in the method for producing the optical filter according to the embodiment of the present disclosure has the same definition as the support in the laminate according to the embodiment of the present disclosure, and the same applies to preferred aspects thereof, so the description thereof will not be repeated here.

Examples of the support include a semiconductor substrate such as silicon. In addition, examples of the support include those described in the section of the laminate according to the embodiment of the present disclosure.

For example, an organic film or an inorganic film may be formed on the support.

Examples of a material of the organic film include the resins described above.

For example, a charge coupled device (CCD), a complementary metal-oxide semiconductor (CMOS), or a transparent conductive film may be formed on the support.

A black matrix that separates pixels from each other may be formed on the support.

If necessary, the support may be provided with an undercoat layer in order to improve the adhesiveness with an adjacent layer, prevent the diffusion of substances, or flatten the surface of the support.

In a case where a support made of glass (so-called glass substrate) is used as the support, it is preferable to form an inorganic film on the surface of the glass substrate or to subject the glass substrate to a dealkalization treatment before use.

The method of applying the composition according to the embodiment of the present disclosure onto a support is not particularly limited, and a known application method can be adopted.

Examples of the application method include a dropping method (so-called drop casting method); a slit coating method; a spray coating method; a roll coating method; a rotary coating method (so-called spin coating method); a cast coating method; a slit and spin method; a pre-wetting method (for example, a coating method described in JP2009-145395A); various printing methods including a jet printing method such as an ink jet (for example, an on-demand method, a piezoelectric method, or a thermal method) or a nozzle jet, a flexographic printing, a screen printing, a gravure printing, a reverse offset printing, and a metal mask printing; a transfer method using a mold or the like; and a nanoimprinting method.

The composition layer formed on the support may be subjected to a drying treatment (pre-baking).

In a case where the pre-baking is carried out, the pre-baking temperature is not particularly limited and is, for example, preferably 150° C. or lower, more preferably 120° C. or lower, and still more preferably 110° C. or lower. In a case where the pre-baking temperature is 150° C. or lower, and then for example, in a case where a photoelectric conversion film of an image sensor is formed of an organic material, the characteristics of the organic material can be maintained more effectively.

The lower limit of the pre-baking temperature can be set to, for example, 50° C. or higher, or can also be set to 80° C. or higher.

The pre-baking time is not particularly limited and is, for example, preferably 10 seconds to 3000 seconds, more preferably 40 seconds to 2500 seconds, and still more preferably 80 seconds to 220 seconds.

The pre-baking unit is not particularly limited, and examples thereof include a hot plate and a convection oven (so-called hot air circulation type dryer).

A heat treatment (post-baking) may be further carried out after the pre-baking.

In a case where the post-baking is carried out, the post-baking temperature is not particularly limited and is, for example, preferably 100° C. to 240° C. From the viewpoint of film curing, for example, the post-baking temperature is more preferably 180° C. to 240° C.

The post-baking time is not particularly limited and is, for example, preferably 2 minutes to 10 minutes and more preferably 4 minutes to 8 minutes.

The post-baking unit is not particularly limited, and examples thereof include a hot plate and a convection oven (so-called hot air circulation type dryer).

In a case where the optical filter according to the embodiment of the present disclosure is produced, and then in a case where the composition according to the embodiment of the present disclosure which is a curable composition is used, it is preferable that the method for producing the optical filter according to the embodiment of the present disclosure includes a step of subjecting the composition layer formed above to a curing treatment.

In a case where the composition layer is subjected to a curing treatment, the mechanical strength of the optical filter can be improved.

The curing treatment is not particularly limited, and examples thereof include treatments such as a full-surface exposure treatment and a full-surface heating treatment.

The “exposure” in the present disclosure includes not only irradiation with light of various wavelengths but also irradiation with radiation such as an electron beam or an X-ray.

The exposure is preferably carried out by irradiation with radiation.

The radiation is not particularly limited, and is particularly preferably an electron beam, an ultraviolet ray such as KrF, ArF, a g-line, an h-line, or an i-line, visible light, or the like.

The exposure method is not particularly limited, and examples thereof include stepper exposure and exposure using a high-pressure mercury lamp.

The exposure amount is not particularly limited and is, for example, preferably 5 mJ/cm² to 3000 mJ/cm². The lower limit of the exposure amount is more preferably 10 mJ/cm² or more and still more preferably 50 mJ/cm² or more. The upper limit of the exposure amount is more preferably 2000 mJ/cm² or less and still more preferably 1000 mJ/cm² or less.

The method for producing the optical filter according to the embodiment of the present disclosure may include a step of forming a pattern.

Examples of the pattern forming method include a pattern forming method using a photolithography method and a pattern forming method using a dry etching method.

### [Image Forming Material]

The image forming material according to the embodiment of the present disclosure contains the compound represented by Formula (1) described above or the tautomer thereof (that is, the compound according to the embodiment of the present disclosure).

The image forming material according to the embodiment of the present disclosure can be particularly suitably used as an image recording material for recording an invisible image.

Specific examples of the image forming material include an ink jet recording material, a heat-sensitive recording material, a pressure-sensitive recording material, a recording material using an electrophotographic method, a transfer type silver halide photosensitive material, a printing ink, a recording pen, and a hanko (Japanese Stamp). Above all, the image forming material according to the embodiment of the present disclosure can be particularly suitably used as an ink jet recording material or a recording material using an electrophotographic method.

The image forming material according to the embodiment of the present disclosure preferably contains a liquid medium.

The liquid medium is not particularly limited and for example, the same solvent as in the composition according to the embodiment of the present disclosure can be used. The liquid medium in a case where the image forming material according to the embodiment of the present disclosure is an aqueous composition will be described later.

The image forming material according to the embodiment of the present disclosure may contain the compound according to the embodiment of the present disclosure in a state of being dissolved in a liquid medium or may contain the compound according to the embodiment of the present disclosure in a state of being dispersed as solid particles in a liquid medium.

The image forming material containing the compound according to the embodiment of the present disclosure as solid particles can be prepared by dispersing the compound according to the embodiment of the present disclosure in a liquid medium using a dispersion apparatus.

The dispersion apparatus is not particularly limited, and a known dispersion apparatus in the related art can be used.

Specific examples of the dispersion apparatus include a ball mill, a sand mill, a beads mill, a roll mill, a jet mill, a paint shaker, an attritor, an ultrasonic disperser, and a DISPER.

The volume average particle size of the particles is not particularly limited and is, for example, preferably 10 nm to 250 nm, more preferably 20 nm to 250 nm, and still more preferably 30 nm to 230 nm.

In a case where the volume average particle size of the particles is within the above-described range, the preservation stability of the image forming material is further improved and a sufficient optical density can be obtained.

The volume average particle size of the particles is measured using a particle size distribution measuring device that adopts a dynamic light scattering method. For example, a particle size distribution measuring device (trade name: UPA-EX150, manufactured by MicrotracBEL Corp.) can be used as the measuring device. In this regard, the measuring device is not limited thereto.

The image forming material according to the embodiment of the present disclosure may contain only one type of the compound according to the embodiment of the present disclosure, or may contain two or more types of the compounds according to the embodiment of the present disclosure.

The content of the compound according to the embodiment of the present disclosure in the image forming material is not particularly limited, and can be appropriately set depending on the intended purpose. In general, from the viewpoint of sufficiently exhibiting an infrared absorbing ability, the content of the compound according to the embodiment of the present disclosure in the image forming material is preferably 0.001% by mass to 30% by mass, more preferably 0.01% by mass to 10% by mass, and still more preferably 0.05% by mass to 5% by mass with respect to the total mass of the image forming material.

In a case where the image forming material according to the embodiment of the present disclosure is an aqueous composition, examples of the liquid medium include water and a mixed liquid of water and an organic solvent.

The content of water in the liquid medium is preferably 30% by mass to 100% by mass and more preferably 50% by mass to 100% by mass with respect to the total mass of the liquid medium.

The water is not particularly limited and is, for example, preferably distilled water, ion exchange water, ion exchange distilled water, or pure water from the viewpoint of containing few impurities.

In a case where the liquid medium contains an organic solvent other than water, examples of the organic solvent include an alcohol compound such as methanol, ethanol, propanol, isopropanol, butanol, isobutanol, sec-butanol, t-butanol, pentanol, hexanol, cyclohexanol, or benzyl alcohol; a polyhydric alcohol compound such as ethylene glycol, diethylene glycol, triethylene glycol, polyethylene glycol, propylene glycol, dipropylene glycol, polypropylene glycol, butylene glycol, hexanediol, pentanediol, glycerin, hexanetriol, or thiodiglycol; a glycol derivative such as ethylene glycol monomethyl ether, ethylene glycol monoethyl ether, ethylene glycol monobutyl ether, diethylene glycol monomethyl ether, diethylene glycol monobutyl ether, propylene glycol monomethyl ether, propylene glycol monoethyl ether, propylene glycol monobutyl ether, dipropylene glycol monomethyl ether, triethylene glycol monomethyl ether, ethylene glycol diacetate, ethylene glycol monomethyl ether acetate, triethylene glycol monoethyl ether, ethylene glycol monophenyl ether, 3-methyl-3-methoxybutanol, or 3-methoxybutanol; an amine compound such as ethanolamine, diethanolamine, triethanolamine, N-methyl diethanolamine, N-ethyl diethanolamine, morpholine, N-ethyl morpholine, ethylene diamine, diethylene triamine, triethylene tetramine, polyethyleneimine, or tetramethyl propylene diamine; and an organic solvent that is soluble in water, such as formamide, N,N-dimethyl formamide, N,N-dimethyl acetamide, dimethyl sulfoxide, sulfolane, 2-pyrrolidone, N-methyl-2-pyrrolidone, N-vinyl-2-pyrrolidone, 2-oxazolidone, 1,3-dimethyl-2-imidazolidinone, acetonitrile, acetone, methyl ethyl ketone, tetrahydrofuran, or butyl cellosolve.

In a case where the image forming material according to the embodiment of the present disclosure is an aqueous composition, the image forming material according to the embodiment of the present disclosure may further contain an aqueous resin.

Examples of the aqueous resin include a resin that dissolves in water, a water-dispersible resin that disperses in water, a colloidal dispersion resin, and a mixture thereof.

Here, the term “resin that dissolves in water” refers to a resin that dissolves in an amount of 1% by mass or more in water at 25° C. Specific examples of the resin that dissolves in water include gelatin, a vinyl resin (for example, polyvinyl alcohol), and a water-soluble cellulose derivative (for example, carboxymethyl cellulose).

Examples of the water-dispersible resin include a hydrophobic synthetic resin.

Specific examples of the water-dispersible resin include an acrylic resin, a styrene-acrylic resin, a vinyl resin, a polyurethane, a polyester, a polyamide, and a fluororesin.

Examples of the acrylic resin include a homopolymer or copolymer obtained by polymerization of at least one monomer selected from the group consisting of an acrylic acid, an acrylic acid ester compound (for example, an alkyl acrylate), an acrylamide, an acrylonitrile, a methacrylic acid, a methacrylic acid ester compound (for example, an alkyl methacrylate), a methacrylamide, and a methacrylonitrile.

Above all, the acrylic resin is preferably a homopolymer or copolymer obtained by polymerization of at least one monomer selected from the group consisting of an acrylic acid ester compound and a methacrylic acid ester compound, and more preferably a homopolymer or copolymer obtained by polymerization of at least one monomer selected from the group consisting of an acrylic acid ester compound having an alkyl group having 1 to 6 carbon atoms and a methacrylic acid ester compound having an alkyl group having 1 to 6 carbon atoms.

In a case where the image forming material according to the embodiment of the present disclosure further contains an aqueous resin, the image forming material according to the embodiment of the present disclosure may contain the aqueous resin in the form of an aqueous dispersion of resin particles.

A commercially available product can be used as the aqueous dispersion of resin particles.

Examples of commercially available products of the aqueous dispersion of resin particles include SUPERFLEX 830, 460, 870, 420, and 420NS [manufactured by DKS Co. Ltd.; polyurethane], BONDIC 1370NS and 1320NS [manufactured by DIC Corporation; polyurethane], HYDRAN HW140SF, WLS201, WLS202, and WLS213 [manufactured by DIC Corporation; polyurethane], OLESTER UD350, UD500, and UD600 [manufactured by Mitsui Chemicals, Inc.; polyurethane], NEOREZ R972, R966, and R9660 [manufactured by Kusumoto Chemicals, Ltd.; polyurethane], FINETEX ES650 and ES2200 [manufactured by DIC Corporation; polyester], VYLONAL (registered trademark) MD1100, MD1400, and MD1480 [manufactured by Toyobo Co., Ltd.; polyester], JURYMER (registered trademark) ET325, ET410, AT-613, and SEK301 [manufactured by Nihon Junyaku Co., Ltd.; acrylic resin], BONCOAT AN117 and AN226 [manufactured by DIC Corporation; acrylic resin], LACSTAR DS616 and DS807 [manufactured by DIC Corporation; styrene-butadiene rubber], NIPOL LX110, LX206, LX426, and LX433 [manufactured by Zeon Corporation; styrene-butadiene rubber], and NIPOL LX513, LX1551, LX550, and LX1571 [manufactured by Zeon Corporation; acrylonitrile-butadiene rubber].

The image forming material according to the embodiment of the present disclosure preferably further contains a surfactant.

In a case where the image forming material according to the embodiment of the present disclosure further contains a surfactant, for example, the dispersibility of the particles can be improved. In addition, in a case where the image forming material according to the embodiment of the present disclosure further contains a surfactant, for example, the quality of the formed image can be improved.

The surfactant in the image forming material according to the embodiment of the present disclosure has the same definition as the surfactant in the composition according to the embodiment of the present disclosure, and the same applies to preferred aspects thereof, so the description thereof will not be repeated here.

In a case where the image forming material according to the embodiment of the present disclosure is an ink, the image forming material according to the embodiment of the present disclosure contains the compound according to the embodiment of the present disclosure and a liquid medium.

Specific examples of the ink include an ink for planographic printing, an ink for ink jetting, an ultraviolet curable ink, an ink for writing instruments (for example, a ballpoint pen), a toner, an ink for vermilion, an ink for penetrating stamps, a textile printing ink, an ink for letterpress printing, an ink for intaglio printing (for example, gravure printing), an ink for stencil printing (for example, screen printing), and a flexographic ink.

In a case where the image forming material according to the embodiment of the present disclosure is an ink, the compound according to the embodiment of the present disclosure is preferably in a state of being dispersed as solid particles in a liquid medium, and the liquid medium is preferably water or a mixed liquid of water and an organic solvent.

In a case where the image forming material according to the embodiment of the present disclosure is an ink, the image forming material according to the embodiment of the present disclosure may contain various additives as necessary as long as the effect of the present disclosure is not impaired.

Examples of the additive include additives such as a resin, an anti-drying agent (so-called wetting agent), an antifading agent, an emulsification stabilizer, a penetration enhancer, a preservative, a fungicide, a pH adjuster, a surface tension adjuster, an antifoaming agent, a viscosity adjuster, a dispersant, a dispersion stabilizer, a rust inhibitor, and a chelating agent.

The antifading agent is used for the purpose of improving the storage stability of an image formed by an ink which is the image forming material according to the embodiment of the present disclosure.

Examples of the resin include the same resins as those in the composition according to the embodiment of the present disclosure.

In a case where the image forming material according to the embodiment of the present disclosure is an aqueous composition, the additive can be directly contained in the image forming material according to the embodiment of the present disclosure.

The recording medium in a case where an image is formed using the image forming material according to the embodiment of the present disclosure is not particularly limited, and examples thereof include paper such as ordinary uncoated paper and coated paper, a resin film obtained by forming various non-absorbent resin materials used in so-called soft packaging into a film, and a metal foil.

Specific examples of the paper include pure white roll paper, kraft paper, paperboard, high-quality paper, OCR paper, art paper, coated paper, mirror-coated paper, condenser paper, and paraffin paper.

Specific examples of the resin film include a polyester film, a polypropylene (PP) film, a cellophane, an acetate film, a polycarbonate (PC) film, an acrylic resin film, a polyethylene terephthalate (PET) film, a biaxially oriented polystyrene (OPS) film, a biaxially oriented polypropylene (OPP) film, a biaxially oriented nylon (ONy) film, a polyvinyl chloride (PVC) film, a polyethylene (PE) film, and a triacetate (TAC) film.

In addition, examples of the recording medium include laminated paper obtained by coating paper with a resin and a composite substrate obtained by forming a metal layer of copper, aluminum, or the like on paper or a resin film.

### EXAMPLES

Hereinafter, the present disclosure will be described in detail with reference to Examples. However, the present disclosure is not limited to the following Examples.

### [Synthesis of Compound]

### Example 1: Synthesis of Compound Y-2

The compound Y-2 was synthesized as follows.

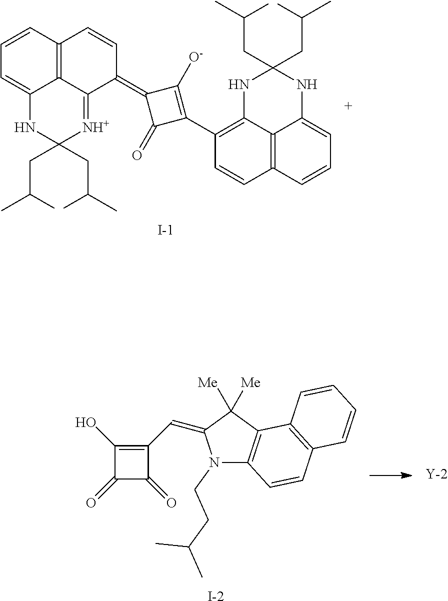

645 mg of compound I-1, 376 mg of compound I-2, 5 mL of n-butanol, and 5 mL of toluene were added to a 50 mL eggplant flask. This was followed by heating under reflux for 4 hours while removing produced water using a Dean-Stark trap. The reaction mixture was cooled to 22° C. and concentrated under reduced pressure. 20 mL of methanol was added to the residue, and the precipitated crystals were collected by filtration. Recrystallization was carried out using 10 mL of acetonitrile to obtain 169 mg of the compound Y-2.

The compound Y-2 was confirmed to have the following structure by ¹H-NMR.

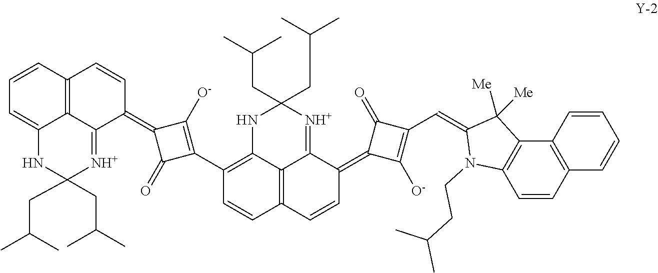 [SMILES: CC(C)CCN1/C(=C\C2=C([O-])C(=c3\ccc4ccc(C5=C([O-])/C(=c6\ccc7cccc8c7c6=[NH+]C(CC(C)C)(CC(C)C)N8)C5=O)c5c4c3=[NH+]C(CC(C)C)(CC(C)C)N5)/C2=O)C(C)(C)c2c1ccc1ccccc21]

The NMR data of the compound Y-2 is shown below.

### (Compound Y-2)

¹H-NMR (CDCl₃) δ=10.89 (br.s, 1H), 10.40 (br.s, 1H), 10.11 (br.s, 1H), 8.45 (s, 1H), 8.22 (t, 1H), 8.07 (d, 1H), 8.01-7.91 (m, 3H), 7.66 (t, 1H), 7.53 (t, 1H), 7.38 (d, 1H), 7.31 (t, 1H), 6.85 (d, 1H), 6.75 (d, 1H), 6.73-6.64 (m, 2H), 6.38 (d, 1H), 6.09 (s, 1H), 4.35-4.16 (m, 3H), 2.11-1.69 (m, 15H), 2.01 (s, 6H), 1.16-0.92 (m, 30H)

### Example 2: Synthesis of Compound Y-44

The compound Y-44 was synthesized as follows.

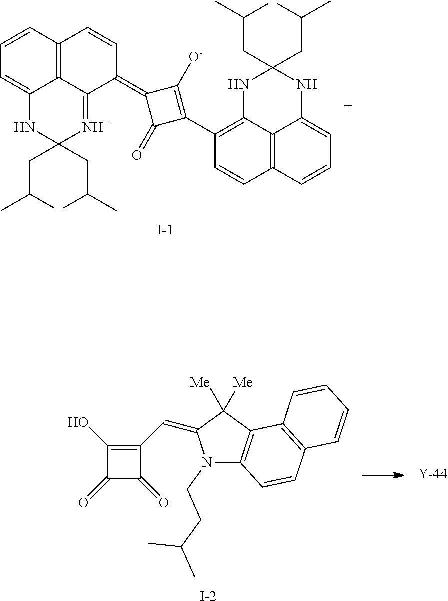

672 mg of the compound I-1, 566 mg of the compound I-2, 6 mL of n-butanol, and 6 mL of toluene were added to a 50 mL eggplant flask. This was followed by heating under reflux for 6 hours while removing produced water using a Dean-Stark trap. The reaction mixture was cooled to 23° C. and concentrated under reduced pressure. The residue was purified by silica gel chromatography to obtain 406 mg of the compound Y-44.

The compound Y-44 was confirmed to have the following structure by ¹H-NMR.

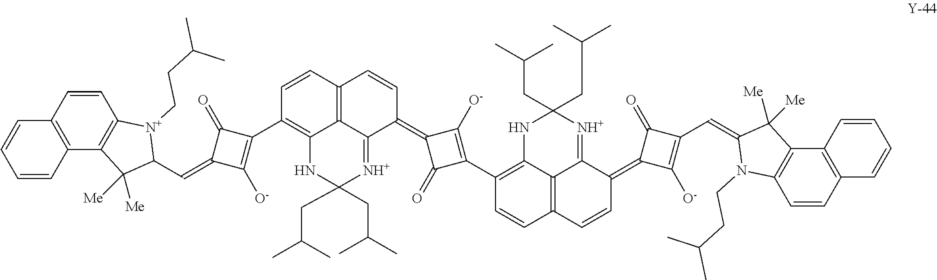 [SMILES: CC(C)CCN1/C(=C\C2=C([O-])C(=c3\ccc4ccc(C5=C([O-])/C(=c6\ccc7ccc(C8=C([O-])/C(=C/C9[NH+](CCC(C)C)c%10ccc%11ccccc%11c%10C9(C)C)C8=O)c8c7c6=[NH+]C(CC(C)C)(CC(C)C)N8)C5=O)c5c4c3=[NH+]C(CC(C)C)(CC(C)C)N5)/C2=O)C(C)(C)c2c1ccc1ccccc21]

The NMR data of the compound Y-44 is shown below.

### (Compound Y-44)

¹H-NMR (CDCl₃) δ=10.50 (br.s, 2H), 9.88 (br.s, 2H), 8.29-7.80 (m, 12H), 7.61 (t, 2H), 7.48 (t, 2H), 7.35 (d, 2H), 6.61 (d, 2H), 6.16 (s, 2H), 4.29 (t, 4H), 2.17-1.60 (m, 18H), 1.99 (s, 12H), 1.10-0.90 (m, 36H)

### Example 3: Synthesis of Compound Y-35

The compound Y-35 was synthesized as follows.

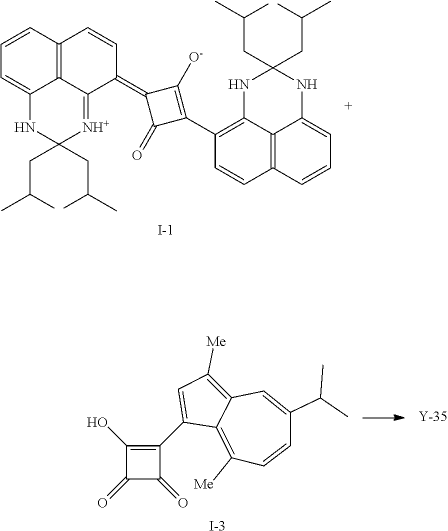

643 mg of the compound I-1, 305 mg of compound I-3, 4 mL of n-butanol, and 6 mL of toluene were added to a 50 mL eggplant flask. This was followed by heating under reflux for 3 hours while removing produced water using a Dean-Stark trap. The reaction mixture was cooled to 26° C. and concentrated under reduced pressure. The residue was purified by silica gel chromatography to obtain 360 mg of the compound Y-35.

The compound Y-35 was confirmed to have the following structure by ¹H-NMR.

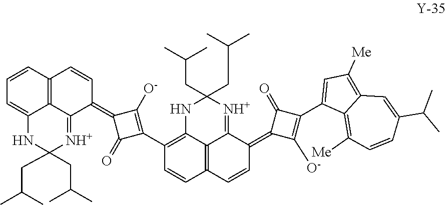 [SMILES: Cc1cc(C2=C([O-])/C(=c3\ccc4ccc(C5=C([O-])/C(=c6\ccc7cccc8c7c6=[NH+]C(CC(C)C)(CC(C)C)N8)C5=O)c5c4c3=[NH+]C(CC(C)C)(CC(C)C)N5)C2=O)c2c(C)ccc(C(C)C)cc1-2]

The NMR data of the compound Y-35 is shown below.

### (Compound Y-35)

¹H-NMR (CDCl₃) δ=11.17 (br.s, 1H), 10.91 (s, 1H), 10.19 (br.s, 1H), 8.60 (s, 1H), 8.32 (d, 1H), 8.16 (d, 1H), 8.09 (s, 1H), 7.94 (d, 1H), 7.58 (d, 1H), 7.47 (d, 1H), 7.32 (t, 1H), 6.82 (d, 1H), 6.74 (d, 1H), 6.71-6.61 (m, 2H), 6.40 (s, 1H), 4.33 (br.s, 1H), 3.33 (s, 3H), 3.12 (m, 1H), 2.54 (s, 3H), 2.23-1.74 (m, 12H), 1.38 (d, 6H), 1.14-0.95 (m, 24H)

### Example 4: Synthesis of Compound Y-55

The compound Y-55 was synthesized as follows.

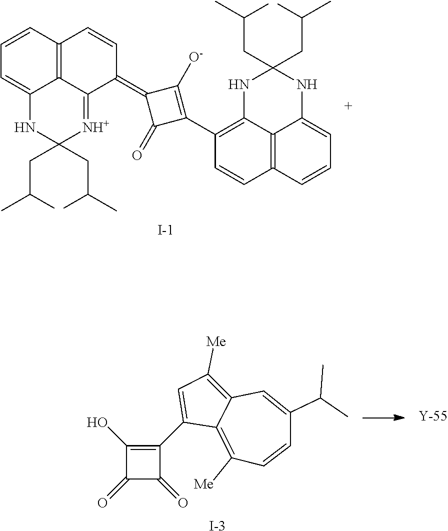

644 mg of the compound I-1, 652 mg of the compound I-3, 4 mL of n-butanol, and 8 mL of toluene were added to a 50 mL eggplant flask. This was followed by heating under reflux for 2 hours while removing produced water using a Dean-Stark trap. Further, 242 mg of the compound I-3 was added thereto, and heating under reflux was continued for 6 hours while removing produced water. The reaction mixture was cooled to 22° C. and concentrated under reduced pressure. The residue was purified by silica gel chromatography to obtain 73 mg of the compound Y-55.

The compound Y-55 was confirmed to have the following structure by ¹H-NMR.

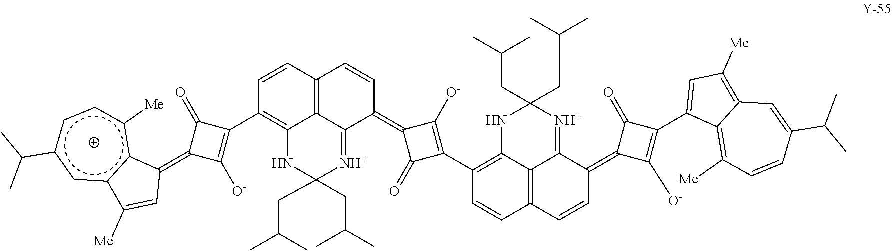

The NMR data of the compound Y-55 is shown below.

### (Compound Y-55)

¹H-NMR (CDCl₃) δ=10.76 (br.s, 2H), 10.61 (br.s, 2H), 8.64 (s, 2H), 8.40 (d, 2H), 8.15-7.96 (m, 4H) 7.69-7.51 (m, 4H), 6.72-6.63 (m, 4H), 3.35 (s, 6H), 3.15 (m, 2H), 2.55 (s, 6H), 2.23-1.91 (m, 12H), 1.39 (d, 12H), 1.06 (d, 24H)

### Comparative Example 1: Comparative Compound R-1

The comparative compound R-1 having the following structure was used.

### Comparative Example 2: Comparative Compound R-2

The comparative compound R-2 having the following structure was used.

### Comparative Example 3: Comparative Compound R-3

The comparative compound R-3 having the following structure was used.

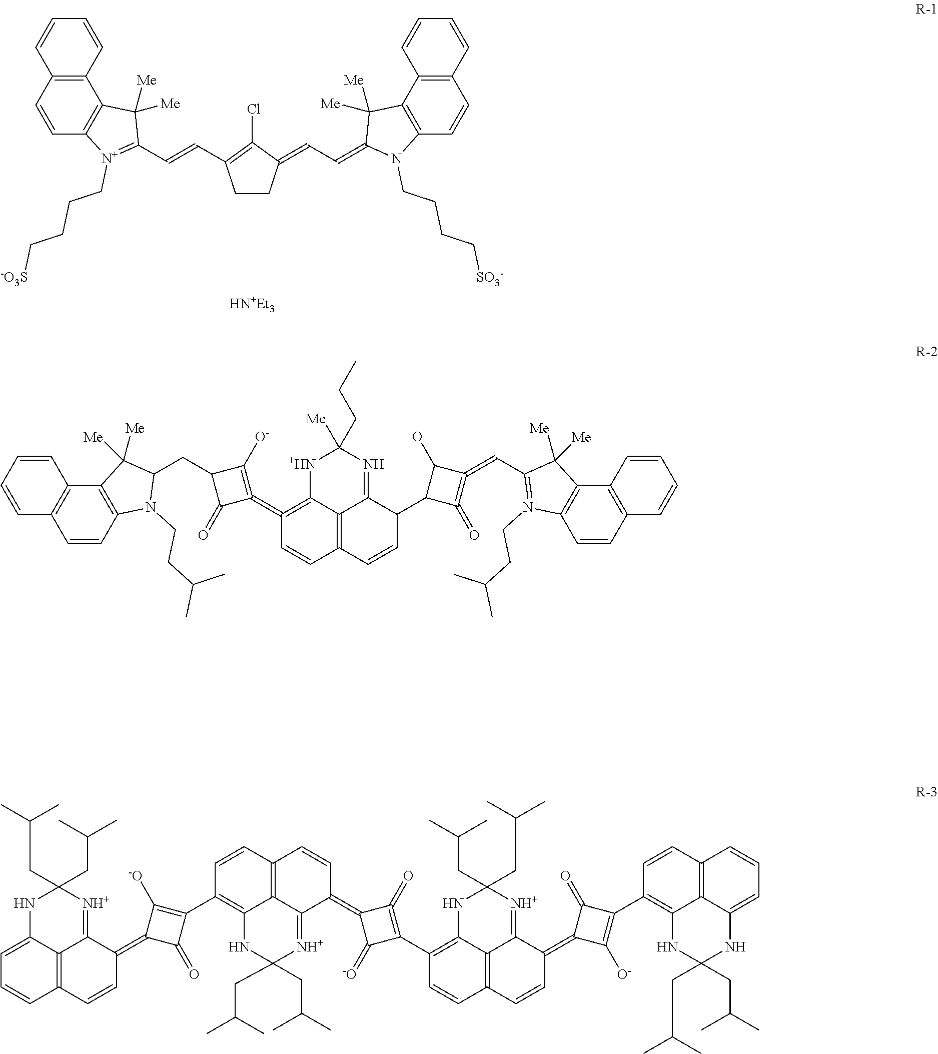

### <Spectral Characteristics>

The absorption spectrum of each of the compounds of Examples 1 to 4 and Comparative Examples 1 to 3 was measured according to the following procedure, and the maximal absorption wavelength (λmax) and the molar absorption coefficient (ε) were determined. The results are shown in Table 1.

Using chloroform as a solvent, a solution was prepared in which the compound was dissolved so that the concentration of the compound in chloroform was 5×10⁻⁶ mol/L (liter; the same applies hereinafter). Each of the prepared solutions was placed in a 1 cm cell, and the absorption spectrum was measured using a spectrophotometer [trade name: UV-3600, manufactured by Shimadzu Corporation] as a measuring device.

TABLE 1

| | | | | ε |
|---|---|---|---|---|
| | Compound | λmax | | [L/(mol · cm)] |
| Example 1 | Compound Y-2 | 944 | nm | 166,000 |
| Example 2 | Compound Y-44 | 1049 | nm | 310,000 |
| Example 3 | Compound Y-35 | 989 | nm | 246,000 |
| Example 4 | Compound Y-55 | 1122 | nm | 301,000 |
| Comparative | Comparative | 861 | nm | 301,000 |
| Example 1 | compound R-1 | | | |
| Comparative | Comparative | 884 | nm | 254,000 |
| Example 2 | compound R-2 | | | |
| Comparative | Comparative | 1121 | nm | 300,000 |
| Example 3 | compound R-3 | | | |

### [Production of Laminate]

### Example 5

7.6 g of chloroform was added to 4.2 mg of the compound Y-2 and 1.1 g of DIANAL (registered trademark) BR-80 [type of resin: polymethyl methacrylate (PMMA), manufactured by Mitsubishi Chemical Corporation] which were then mixed under stirring at room temperature for 30 minutes to obtain a resin composition. The obtained resin composition was applied by spin coating onto a glass plate as a transparent support, and dried at 100° ° C. for 2 minutes. In this manner, a laminate including a film (thickness: 10 μm) which is a cured product of a resin composition on a transparent support was obtained.

### Example 6

A laminate including a film (thickness: 10 μm) which is a cured product of a resin composition on a transparent support was obtained in the same manner as in Example 5, except that 3.1 mg of the compound Y-44 was used instead of 4.2 mg of the compound Y-2 in Example 5.

### Example 7

A laminate including a film (thickness: 10 μm) which is a cured product of a resin composition on a transparent support was obtained in the same manner as in Example 5, except that 2.6 mg of the compound Y-35 was used instead of 4.2 mg of the compound Y-2 in Example 5.

### Example 8

A laminate including a film (thickness: 10 μm) which is a cured product of a resin composition on a transparent support was obtained in the same manner as in Example 5, except that 3.1 mg of the compound Y-55 was used instead of 4.2 mg of the compound Y-2 in Example 5.

### Comparative Example 4

A laminate including a film (thickness: 10 μm) which is a cured product of a resin composition on a transparent support was obtained in the same manner as in Example 5, except that 2.1 mg of the comparative compound R-1 was used instead of 4.2 mg of the compound Y-2 in Example 5.

### Comparative Example 5

A laminate including a film (thickness: 10 μm) which is a cured product of a resin composition on a transparent support was obtained in the same manner as in Example 5, except that 2.6 mg of the comparative compound R-2 was used instead of 4.2 mg of the compound Y-2 in Example 5.

### Comparative Example 6

A laminate including a film (thickness: 10 μm) which is a cured product of a resin composition on a transparent support was obtained in the same manner as in Example 5, except that 2.6 mg of the comparative compound R-3 was used instead of 4.2 mg of the compound Y-2 in Example 5.

### [Evaluation]

### <Light Resistance>

The film included in each of the laminates of Examples 5 to 8 and Comparative Examples 4 to 6 was irradiated with light under the following conditions to evaluate the light resistance. Specifically, the following operations were carried out.

The absorbance at the maximal absorption wavelength (λmax) of the film included in the laminate (hereinafter, referred to as “absorbance at λmax before irradiation with light”) was measured using a spectrophotometer [trade name: UV-3600, manufactured by Shimadzu Corporation] as a measuring device.

Next, the film included in the laminate was irradiated with light under the following conditions, and the absorbance at the maximal absorption wavelength (λmax) of the film one hour after irradiation with light (hereinafter, referred to as “absorbance at λmax after irradiation with light”) was measured using a spectrophotometer [trade name: UV-3600, manufactured by Shimadzu Corporation] as a measuring device.

The residual rate (%) of absorbance was calculated from the absorbance at λmax before irradiation with light and the absorbance at λmax after irradiation with light based on the following expression. The results are shown in Table 2.

The higher the residual rate (%) of absorbance, the higher the residual rate of the compound contained in the film (for example, the compound Y-2 in Example 5), indicating that the compound has excellent light resistance.

Residual rate (%) of absorbance=[absorbance at λmax after irradiation with light/absorbance at λmax before irradiation with light]×100

### —Conditions—

Test device: Xenon Weather Meter XL75Z [trade name, manufactured by Suga Test Instruments Co., Ltd.]
Irradiance: 10 klx (40 w/m²)
Irradiation time: 1 hour
Temperature inside tank: 23° ° C.
Humidity inside tank: 5% RH

In addition, the color of the laminate before irradiation with light was visually observed, and the absorbance at 500 nm of the laminate before irradiation with light was measured. The results are shown in Table 2.

TABLE 2

| | | | | | Absorbance |
|---|---|---|---|---|---|
| | | | | | at 500 nm |
| | | | Residual | Color of laminate | (before |
| | | | rate of | (before irradiation | irradiation |
| | λmax | | absorbance | with light) | with light) |
| Example 5 | 943 | nm | 54% | Almost colorless | 0.018 |
| Example 6 | 1050 | nm | 87% | Very light green | 0.019 |
| Example 7 | 995 | nm | 71% | Very light blue | 0.023 |
| Example 8 | 1129 | nm | 94% | Very light green | 0.022 |
| Comparative | 861 | nm | 28% | Very light blue | 0.020 |
| Example 4 | | | | | |
| Comparative | 879 | nm | 45% | Light purple | 0.044 |
| Example 5 | | | | | |
| Comparative | 1125 | nm | 58% | Light green | 0.028 |
| Example 6 | | | | | |

As shown in Table 2, the laminates of Examples 5 to 8, each of which includes a film containing the compound represented by Formula (1), all exhibited high values of the residual rate of absorbance, confirming that the compound represented by Formula (1) had excellent light resistance.

On the other hand, the laminate of Comparative Example 4 including a film containing the comparative compound R-1 and the laminate of Comparative Example 5 including a film containing the comparative compound R-2 both exhibited lower values of the residual rate of absorbance as compared with the laminates of Examples, confirming that the light resistance of the comparative compound R-1 and the comparative compound R-2 was inferior to that of the compound represented by Formula (1).

The laminate of Comparative Example 5 including a film containing the comparative compound R-2 and the laminate of Comparative Example 6 including a film containing the comparative compound R-3 were both more clearly colored than the laminates of Examples, confirming that the comparative compound R-2 and the comparative compound R-3 were inferior in the property of suppressing coloration in a visible range as compared with the compound represented by Formula (1).

The disclosure of JP2021-133007 filed on Aug. 17, 2021 is incorporated herein by reference in its entirety.

All documents, patent applications, and technical standards mentioned in the present specification are incorporated herein by reference to the same extent as if individual documents, patent applications, and technical standards are specifically and individually indicated to be incorporated by reference.

What is claimed is:

1. A compound represented by Formula (1) or a tautomer thereof: 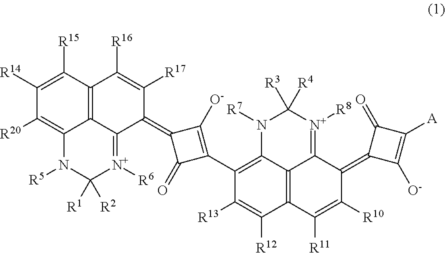
in Formula (1), R¹, R², R³, and R⁴ each independently represent a hydrogen atom, an alkyl group, or an aryl group, R⁵, R⁶, R⁷, and R⁸ each independently represent a hydrogen atom, an alkyl group, or an aryl group, R¹⁰, R¹¹, R¹², R¹³, R¹⁴, R¹⁵, R¹⁶, and R¹⁷ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, R²⁰ represents a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, an arylthio group, or a group represented by Formula (2), and A represents an aromatic hydrocarbon group, a heterocyclic group, or a heterocyclic methine group,
in Formula (2), B represents a cationic aromatic hydrocarbon group, a cationic heterocyclic group, or a cationic heterocyclic methine group, and * represents a bonding position, 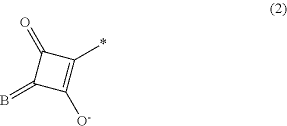
in Formula (1), R¹ and R², and R³ and R⁴ each may be bonded to each other to form a ring, R¹ and R⁵, R² and R⁶, R³ and R⁷, R⁴ and R⁸ each may be bonded to each other to form a ring, and R¹⁰, R¹¹, R¹², R¹³, R¹⁴, R¹⁵, R¹⁶, R¹⁷, and R²⁰ each may be bonded to an adjacent group to form a ring, provided that a compound corresponding to the following [a], a compound corresponding to the following [b], and tautomers thereof are excluded,
[a] in Formula (1), A is a group represented by Formula (P), R²⁰ is a hydrogen atom, R¹ and R³ are the same, and R² and R⁴ are the same,
[b] in Formula (1), A is a group represented by Formula (P), R²⁰ is a group represented by Formula (2), B is a group represented by Formula (Q), R¹ and R³ are the same, and R² and R⁴ are the same, 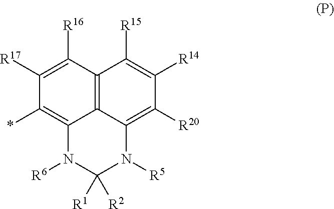
in Formula (P), R¹ and R² each independently represent a hydrogen atom, an alkyl group, or an aryl group, R⁵ and R⁶ each independently represent a hydrogen atom, an alkyl group, or an aryl group, R¹⁴, R¹⁵, R¹⁶, and R¹⁷ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, R²⁰ represents a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, an arylthio group, or a group represented by Formula (2), and * represents a bonding position, 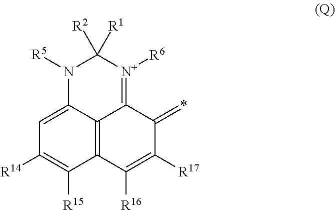
in Formula (Q), R¹ and R² each independently represent a hydrogen atom, an alkyl group, or an aryl group, R⁵ and R⁶ each independently represent a hydrogen atom, an alkyl group, or an aryl group, R¹⁴, R¹⁵, R¹⁶, and R¹⁷ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, and * represents a bonding position.

2. The compound or tautomer thereof according to claim 1,
wherein A in Formula (1) is a group represented by Formula (A-1), a group represented by Formula (A-2), a group represented by Formula (A-3), a group represented by Formula (A-4), or a group represented by Formula (A-5), 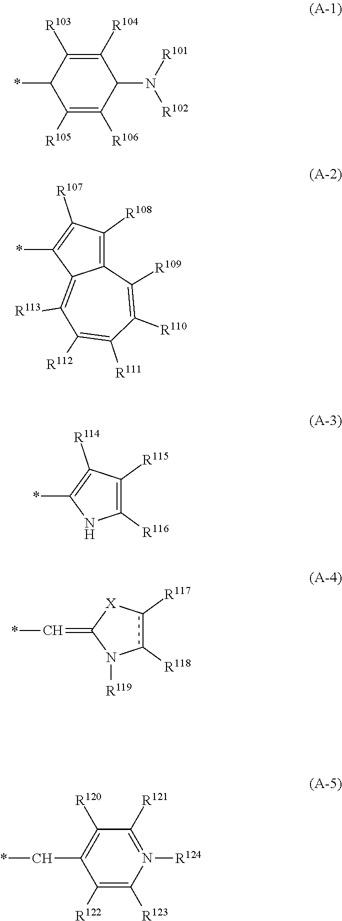
in Formula (A-1), Formula (A-2), Formula (A-3), Formula (A-4), and Formula (A-5), R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹, R¹¹², R¹¹³, R¹¹⁴, R¹¹⁵, R¹¹⁶, R¹¹⁷, R¹¹⁸, R¹¹⁹, R¹²⁰, R¹²¹, R¹²², R¹²³, and R¹²⁴ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, X represents —O—, —S—, —CH═CH—, or —CR¹²⁵R¹²⁶— where R¹²⁵ and R¹²⁶ each independently represent an alkyl group, a bond between the carbon atom to which R¹¹⁷ is bonded and the carbon atom to which R¹¹⁸ is bonded represents a single bond or a double bond, * represents a bonding position, and R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹, R¹¹², R¹¹³, R¹¹⁴, R¹¹⁵, R¹¹⁶, R¹¹⁷, R¹¹⁸, R¹¹⁹, R¹²⁰, R¹²¹, R¹²², R¹²³, R¹²⁴, R¹²⁵, and R¹²⁶ each may be bonded to an adjacent group to form a ring.

3. The compound or tautomer thereof according to claim 1,
wherein B in Formula (2) is a group represented by Formula (B-1), a group represented by Formula (B-2), a group represented by Formula (B-3), a group represented by Formula (B-4), or a group represented by Formula (B-5), 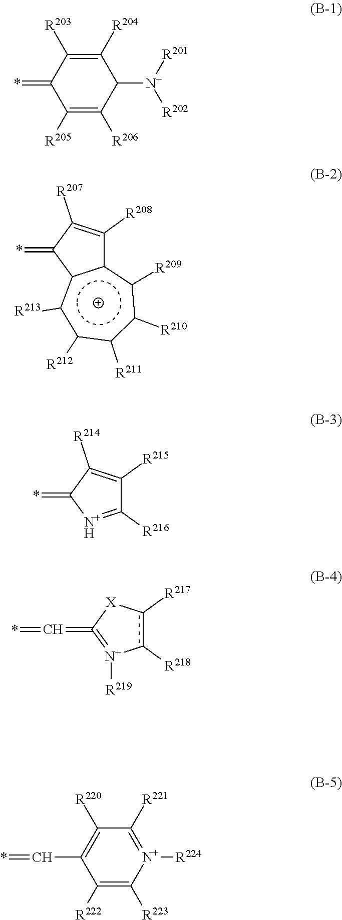
in Formula (B-1), Formula (B-2), Formula (B-3), Formula (B-4), and Formula (B-5), R²⁰¹, R²⁰², R²⁰³, R²⁰⁴, R²⁰⁵, R²⁰⁶, R²⁰⁷, R²⁰⁸, R²⁰⁹, R²¹⁰, R²¹¹, R²¹², R²¹³, R²¹⁴, R²¹⁵, R²¹⁶, R²¹⁷, R²¹⁸, R²¹⁹, R²²⁰, R²²¹, R²²², R²²³, and R²²⁴ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, X represents —O—, —S—, —CH═CH—, or —CR²²⁵R²²⁶— where R²²⁵ and R²²⁶ each independently represent an alkyl group, a bond between the carbon atom to which R²¹⁷ is bonded and the carbon atom to which R²¹⁸ is bonded represents a single bond or a double bond, * represents a bonding position, and R²⁰¹, R²⁰², R²⁰³, R²⁰⁴, R²⁰⁵, R²⁰⁶, R²⁰⁷, R²⁰⁸, R²⁰⁹, R²¹⁰, R²¹¹, R²¹², R²¹³, R²¹⁴, R²¹⁵, R²¹⁶, R²¹⁷, R²¹⁸, R²¹⁹, R²²⁰, R²²¹, R²²², R²²³, R²²⁴, R²²⁵, and R²²⁶ each may be bonded to an adjacent group to form a ring.

4. The compound or tautomer thereof according to claim 1,
wherein R⁵, R⁶, R⁷, and R⁸ in Formula (1) each represent a hydrogen atom.

5. The compound or tautomer thereof according to claim 1,
wherein R¹⁰, R¹¹, R¹², R¹³, R¹⁴, R¹⁵, R¹⁶, and R¹⁷ in Formula (1) each represent a hydrogen atom.

6. The compound or tautomer thereof according to claim 1,
wherein R²⁰ in Formula (1) represents a hydrogen atom or a group represented by Formula (2).

7. A composition comprising:
the compound or tautomer thereof according to claim 1.

8. The composition according to claim 7, further comprising:
a resin.

9. The composition according to claim 7,
wherein the composition is a curable composition.

10. The composition according to claim 7,
wherein the composition is a near-infrared absorbing material.

11. A laminate comprising:
a support; and
a film that is provided on the support and is formed of the composition according to claim 7.

12. An optical filter comprising:
the compound or tautomer thereof according to claim 1.

13. An image forming material comprising:
the compound or tautomer thereof according to claim 1.

14. A method for producing the compound or tautomer thereof according to claim 1, the method comprising:
reacting a compound represented by Formula (3) with a compound represented by Formula (4), 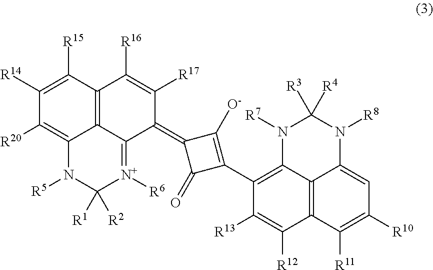
in Formula (3), R¹, R², R³, and R⁴ each independently represent a hydrogen atom, an alkyl group, or an aryl group, R⁵, R⁶, R⁷, and R⁸ each independently represent a hydrogen atom, an alkyl group, or an aryl group, R¹⁰, R¹¹, R¹², R¹³, R¹⁴, R¹⁵, R¹⁶, and R¹⁷ each independently represent a hydrogen atom, a halogen atom, a nitro group, an alkyl group, an aryl group, a hydroxy group, an alkoxy group, an aryloxy group, an acyloxy group, a cyano group, a carboxy group, an alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, or an arylthio group, and R²⁰ represents a hydrogen atom, a halogen atom, a nitro group, an alkyl group, an aryl group, a hydroxy group, alkoxycarbonyl group, a carbamoyl group, an acyl group, an amino group, an amide group, a carbamoylamino group, an alkoxycarbonylamino group, a sulfonamide group, a sulfo group, a sulfamoyl group, an alkylthio group, an arylthio group, or a group represented by Formula (2), 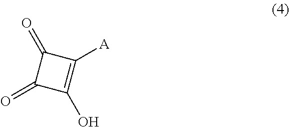
in Formula (4), A represents an aromatic hydrocarbon group, a heterocyclic group, or a heterocyclic methine group.

15. The method according to claim 14,
wherein A in Formula (4) is a group represented by Formula (A-1), R²⁰ in Formula (3) is a group represented by Formula (2), and B in Formula (2) is a group represented by Formula (B-1), or
A in Formula (4) is a group represented by Formula (A-2), R²⁰ in Formula (3) is a group represented by Formula (2), and B in Formula (2) is a group represented by Formula (B-2), or
A in Formula (4) is a group represented by Formula (A-3), R²⁰ in Formula (3) is a group represented by Formula (2), and B in Formula (2) is a group represented by Formula (B-3), or
A in Formula (4) is a group represented by Formula (A-4), R²⁰ in Formula (3) is a group represented by Formula (2), and B in Formula (2) is a group represented by Formula (B-4), or
A in Formula (4) is a group represented by Formula (A-5), R²⁰ in Formula (3) is a group represented by Formula (2), and B in Formula (2) is a group represented by Formula (B-5).
